(19) United States
(12) Patent Application Publication
(10) Pub. No.: US 2007/0060500 A1
(43) Pub. Date: Mar. 15, 2007

(54) Pharmaceutical compositions for prevention of overdose or abuse

(75) Inventors: Travis Mickle, Coralville, IA (US); Suma Krishnan, Belvedere, CA (US); James Scott Moncrief, Christiansburg, VA (US); Christopher Lauderback, Blacksburg, VA (US); Christal Miller, Coralville, IA (US); Thomas Piccariello, Blacksburg, VA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: New River Pharmaceuticals Inc., Radford, VA (US)

(21) Appl. No.: 11/392,878

(22) Filed: Mar. 30, 2006

Related U.S. Application Data
(63) Continuation-in-part of application No. PCT/US04/32131, filed on Sep. 30, 2004.
(63) Continuation-in-part of application No. 10/923,088, filed on Aug. 23, 2004.
Application No. 10/923,088 is a continuation-in-part of application No. 10/156,527, filed on May 29, 2002, now Pat. No. 7,060,708.
Application No. 10/156,527 is a continuation-in-part of application No. 09/987,458, filed on Nov. 14, 2001, now abandoned.
Application No. 10/156,527 is a continuation-in-part of application No. 09/933,708, filed on Aug. 22, 2001, now abandoned.
Application No. 09/933,708 is a continuation-in-part of application No. 09/642,820, filed on Aug. 22, 2000, now Pat. No. 6,716,452.
Application No. 10/156,527 is a continuation-in-part of application No. 09/988,071, filed on Nov. 16, 2001, now abandoned.
Application No. 10/156,527 is a continuation-in-part of application No. 09/988,034, filed on Nov. 16, 2001, now abandoned.
Application No. 10/156,527 is a continuation-in-part of application No. PCT/US01/43089, filed on Nov. 14, 2001.
Application No. 10/156,527 is a continuation-in-part of application No. PCT/US01/43117, filed on Nov. 16, 2001.
(60) Provisional application No. 60/507,012, filed on Sep. 30, 2003.
(60) Provisional application No. 60/567,800, filed on May 5, 2004.
(60) Provisional application No. 60/567,802, filed on May 5, 2004.
(60) Provisional application No. 60/568,011, filed on May 5, 2004.
(60) Provisional application No. 60/248,748, filed on Nov. 16, 2000.
(60) Provisional application No. 60/247,594, filed on Nov. 14, 2000.
(60) Provisional application No. 60/247,684, filed on Nov. 14, 2000.
(60) Provisional application No. 60/248,733, filed on Nov. 16, 2000.
(60) Provisional application No. 60/248,528, filed on Nov. 16, 2000.

(30) Foreign Application Priority Data
Nov. 16, 2001 (WO) PCT/US01/43117
Nov. 16, 2000 (US)

(51) Int. Cl. A61K 38/17

(52) U.S. Cl. 514/2

(57) ABSTRACT

The invention relates to pharmaceutical compositions comprised of a chemical moiety attached to an active agent in a manner that substantially decreases the potential of the active agent to cause overdose or to be abused. When delivered at the proper dosage the pharmaceutical composition provides therapeutic activity similar to that of the parent active agent.

### CROSS REFERENCE RELATED APPLICATIONS

This application claims benefit under 35 U.S.C. 120 and is a continuation-in-part of U.S. PCT/US04/32131 filed Sep. 30, 2004, which claims the benefit under 35 U.S.C. 119(e) to U.S. Provisional application No. 60/567,800 filed May 5, 2004; U.S. Provisional application No. 60/507,012 filed Sep. 30, 2003; U.S. Provisional application No. 60/567,802 filed May 5, 2004; U.S. Provisional application No. 60/568,011 filed on May 5, 2004, all of which are hereby incorporated by reference in their entirety.

This application also claims benefit under 35 U.S.C. 120 as a continuation-in-part of pending U.S. patent application Ser. No. 10/923,257, filed on Aug. 23, 2004, which also claims benefit under 35 U.S.C. 120 as a continuation-in-part of U.S. patent application Ser. No. 10/156,527 filed May 29, 2002, which is a continuation-in-part of U.S. patent application Ser. No. 09/987,458, filed Nov. 14, 2001, now abandoned, which claimed the benefit of each of the following provisional applications under 35 U.S.C. 119(e); U.S. Provisional application No. 60/248,748, filed Nov. 16, 2000, U.S. Provisional application No. 60/247,594, filed Nov. 14, 2000, U.S. Provisional application No. 60/247,684, filed Nov. 14, 2000, and U.S. Provisional application No. 60/248,733, filed Nov. 14, 2000.

U.S. patent application Ser. No. 10/156,527 also claims benefit under 35 U.S.C. 120 as a continuation-in-part of U.S. patent application Ser. No. 09/933,708, filed Aug. 22, 2001, which is hereby incorporated by reference in its entirety, which is a continuation-in-part application of U.S. patent application Ser. No. 09/642,820, filed Aug. 22, 2000, now U.S. Pat. No. 6,716,452 and its divisional application Ser. No. 10/727,565 filed Dec. 5, 2003 which is hereby incorporated by reference in its entirety.

U.S. patent application Ser. No. 10/156,527 also claims benefit under 35 U.S.C. 120 as a continuation-in-part application of U.S. patent application Ser. No. 09/988,071, now abandoned, filed Nov. 16, 2001, which claimed the benefit of the following provisional applications under 35 U.S.C. 119(e);U.S. Provisional application No. 60/248,528, filed Nov. 16, 2000 and U.S. Provisional application No. 60/247,627, filed Nov. 16, 2000, and is also a continuation-in-part application of U.S. patent application Ser. No. 09/988,034, now abandoned, filed Nov. 16, 2001.

U.S. patent application Ser. No. 10/156,527 also claims benefit under 35 U.S.C. 120 as a continuation-in-part application of International Application PCT/US01/43089 designating the U.S., now abandoned, filed Nov. 14, 2001 and International Application PCT/US01/43117 designating the U.S., now abandoned, filed Nov. 16, 2001.

### FIELD OF INVENTION

Accidental and intentional overdose with prescription and over the counter drugs is a serious health problem with thousands of fatalities occurring each year as a result. The present invention relates to pharmaceutical compositions comprised of a chemical moiety attached to an active agent in a manner that substantially decreases the potential of the active agent to cause overdose or to be abused. When delivered at the proper dosage the pharmaceutical composition provides therapeutic activity similar to that of the parent active agent. However, when the composition is delivered at higher doses the potential for overdose or abuse is reduced due to the limited bioavailability of the active agent as compared to the active agent delivered as free drug.

### BACKGROUND

Drug overdose is a significant and growing problem. It can occur accidentally, as when a child swallows pills without understanding the consequences, or intentionally as with suicide attempts. In addition, accidental overdose due to an unusually potent batch of a street drug in illicit drug users is quite common. Common examples of drugs that are seen in overdose cases include the ubiquitous over-the-counter analgesics acetaminophen (paracetamol) and aspirin. While the former is the preferred drug among adolescents in cases of deliberate self poisonings (Lifshitz et al., Isr. Med. Assoc. J., 4(4): 252-4 (2002), aspirin is perhaps more dangerous because there is no antidote (Jones, Am. J. Ther. 9(3):245-57 (2002).

In the elderly population, drugs most often implicated in poisonings include psychotherapeutic drugs, cardiovascular drugs, analgesics and anti-inflammatory drugs, oral hypoglycemics and theophylline (Klein-Schwartz et al., Drugs Aging 1(1):67-89 (1991). It is important to realize that in many cases where death due to overdose is averted, there appears to be extensive morbidity associated with overdoses (Warner-Smith et al., Addition 97(8):963-7 (2002).

The Drug Abuse Warning Network (DAWN) reported in June 2003 on the most recent trends in emergency department (ED) visits related to drug abuse. Data was presented for 8-year trends from 1994 to 2001. The following summaries were provided:
In 2001, there were over 638,000 ED visits related to drug abuse in the conterminous U.S. This translates to 252 visits per 100,000 populations or 0.6 percent of all ED visits.
Seven categories of drugs accounted for 85% of the ED mentions in 2001. The ED visits related to drug abuse most frequently involved alcohol, (34% of mentions), marijuana (17%), benzodiazepines (16%), narcotic analgesic combinations (16%), heroin (15%), other analgesics/combinations (12%), and antidepressants (10%).
ED mentions of benzodiazepines increased 14 percent from 2000 to 2001 (from 91,078 to 103,972), as did the top 2 benzodiazapines, alprazolam (up 16%) and benzodiazepines-NOS (up 35%). The latter includes benzodiazepines not identified by name.
ED mentions of narcotic analgesics/combinations rose 21 percent (from 82,373 to 99,317) from 2000 to 2001.
Narcotic analgesics not identified by name were mentioned most frequently (narcotic analgesics-NOS, 32,196 mentions, up 24% from 2000 to 2001), followed by those containing hydrocodone (21,567), oxycodone (18,409, up 70%), and methadone (10,725, up 37%). Narcotic analgesics/combinations containing propoxyphene (5,361), codeine (3,720, down 30%), and morphine (3,403) were much less frequent and not increasing.

Emergency department reporting for a number of drugs rose substantially from 1994 to 2000. These include: anticonvulsants, including carbamazepine (9,358 to 14,642, up 56.5%), muscle relaxants, including carisoprodol (12,223 to 19,001, up 55.5%), psychotherapeutic drugs, including SSRI antidepressants, tricyclic antidepressants, and other antidepressants (190,467 to 220,289, up 15.7%). Anxiolytics, sedatives, and hypnotics, including benzodiazepines (74,637 to 103,972, up 27.7%) and narcotic analgesics including codeine, hydrocodone, methadone, oxycodone, propoxyphene and others (44,518 to 99,317, up 123.1%).

Other drugs for which the number of ED mentions did not rise but were still responsible for over 10,000 visits include respiratory agents, including antihistamines (12,238), antipsychotics including risperidone (20,182), nonsteroidal anti-inflammatory agents, including ibuprofen and naproxen (22,663) and acetaminophen (42,044). Aspirin and salicylates-NOS accounted for 8,499 ED visits in 2001.

The commercial drugs benzodiazapines (16%), narcotic analgesics other than heroin (16%), non-narcotic analgesics (12%), and antidepressants (10%) accounted for 54% of ED visits in 2001.

Oxycodone is an ingredient of Percodan, Percocet, Roxicet, and Tylox. It is a semisynthetic narcotic analgesic that is derived from thebaine. Available in oral formulations often in combination with aspirin, phenacetin and caffeine. Typical adult dose is 2.5-5 mg as the hydrochloride or terephthalate salt every 6 hours. Although it is typically used for the relief of moderate to moderately severe pain, it can also produce drug dependence of the morphine type. Therapeutic plasma concentration is 10-100 ng/mL and the toxic plasma concentration is greater than 200 ng/mL.

Hydrocodone is an opioid analgesic and antitussive and occurs as fine, white crystals or as crystalline powder. Hydrocodone is a semisynthetic narcotic analgesic prepared from codeine with multiple actions qualitatively similar to those of codeine. It is mainly used as an antitussive in cough syrups and tablets in sub-analgesic doses (2.5-5 mg). Additionally, it is used for the relief of moderate to moderately severe pain. Hydromorphone is administered orally in 5-10 mg doses four times daily. Therapeutic plasma concentration is 1-30 ng/mL and the toxic plasma concentration is greater than 100 ng/mL.

Others have sought to prevent the potential harmful effects of overdose through various formulations. For example, opioids have been combined with antagonists in particular formulations designed to counteract the opioid if the formulation is disrupted before oral administration or is given parenterally. Extended release Concerta (methylphenidate) has been formulated in a paste to preclude administration by snorting or injection. Compositions have been coated with emetics in a quantity that if administered in moderation as intended no emesis occurs, however, if excessive amounts are consumed emesis is induced therefore preventing overdose. These methods, as well as conventional control release formulations, are insufficient and can be easily circumvented. Consequently, improved methods are needed to make drugs with reduced potential for overdose that are resistant to manipulation.

### BRIEF DESCRIPTION OF THE FIGURES

FIG. 1. illustrates preparation of Galacto-Hydrocodone.

FIG. 2. Oral bioavailability of abuse-resistant hydrocodone carbohydrate conjugates, measured as free hydrocodone (with measured plasma levels by ELISA).

FIG. 3. illustrates preparation of Ribo-Hydrocodone.

FIG. 4. Intranasal bioavailability of abuse-resistant hydrocodone carbohydrate conjugate, measured as free hydrocodone (with measured plasma levels by ELISA).

FIG. 5. illustrates preparation of Leu-Hydrocodone.

FIG. 6. illustrates preparation of Ala-Pro-Hydrocodone.

FIG. 7. illustrates the preparation of Gly-Gly-Leu-Hydrocodone.

FIG. 8. illustrates preparation of Gly-Gly-Gly-Gly-Leu-Hydrocodone.

FIG. 9. Intranasal bioavailability of abuse-resistant hydrocodone amino acid, di- and tri-peptide conjugates, measured as free hydrocodone.

FIG. 10. Analgesic effect of abuse-resistant hydrocodone tri-peptide conjugate following intranasal administration, measured as free hydrocodone.

FIG. 11. Analgesic effect of abuse-resistant hydrocodone tri- and penta-peptide conjugates following subcutaneous administration, measured as free hydrocodone.

FIG. 12. Analgesic effect of abuse-resistant hydrocodone penta-peptide conjugate following intransal administration, measured as free hydrocodone.

FIG. 13. Intranasal bioavailability of abuse-resistant hydrocodone tri- and penta-peptide conjugates, measured as free hydrocodone.

FIG. 14. Intranasal bioavailability of abuse-resistant hydrocodone tri- and penta-peptide conjugates, measured as free hydrocodone.

FIG. 15. Intranasal bioavailability of abuse-resistant hydrocodone an amino acid-carbohydrate peptide conjugate, measured as free hydrocodone.

FIG. 16. Analgesic effect of abuse-resistant hydrocodone penta-peptide conjugate following intravenous administration, measured as free hydrocodone.

FIG. 17. Intranasal bioavailability of an abuse-resistant hydrocodone tri-peptide conjugate, measured as free hydrocodone.

FIG. 18. Intranasal bioavailability of an abuse-resistant hydrocodone penta-peptide conjugate, measured as free hydrocodone.

FIG. 19. Intranasal bioavailability of an abuse-resistant hydrocodone tri-peptide conjugate, measured as free hydrocodone.

FIG. 20. Intranasal bioavailability of abuse-resistant hydrocodone tri- and penta-peptide conjugates, measured as free hydrocodone.

FIG. 21. Intranasal bioavailability of abuse-resistant hydrocodone penta-peptide conjugates, measured as free hydrocodone.

FIG. 22. Intranasal bioavailability of an abuse-resistant hydrocodone penta-peptide conjugate, measured as free hydrocodone.

FIG. 23. Intravenous bioavailability of an abuse-resistant hydrocodone tri-peptide conjugate, measured as free hydrocodone.

FIG. 24. Intranasal bioavailability of an abuse-resistant hydrocodone tri-peptide conjugate, measured as free hydrocodone.

FIG. 25. Oral bioavailability of an abuse-resistant hydrocodone penta-peptide conjugate, measured as free hydrocodone.

FIG. 26. Intranasal bioavailability of an abuse-resistant hydrocodone tri-penta-peptide conjugate, measured as free hydrocodone.

FIG. 27. Intranasal bioavailability of an abuse-resistant hydrocodone penta-peptide conjugate, measured as free hydrocodone.

FIG. 28. Intranasal bioavailability of abuse-resistant hydrocodone penta-peptide conjugates, measured as free hydrocodone.

FIG. 29. Intranasal bioavailability of an abuse-resistant hydrocodone tri-peptide conjugate containing D- and L-isomers, measured as free hydrocodone.

FIG. 30. Intranasal bioavailability of an abuse-resistant hydrocodone penta-peptide conjugate, measured as free hydrocodone.

FIG. 31. Intranasal bioavailability of an abuse-resistant hydrocodone penta-peptide conjugate, measured as free hydrocodone.

FIG. 32. Intranasal bioavailability of an abuse-resistant hydrocodone penta-peptide conjugate, measured as free hydrocodone.

FIG. 33. Intranasal bioavailability of abuse-resistant hydrocodone penta-peptide conjugates, measured as free hydrocodone.

FIG. 34. Intranasal bioavailability of an abuse-resistant hydrocodone penta-peptide conjugate, measured as free hydrocodone.

FIG. 35. illustrates preparation of 1,2:3,4-di-O-isopropylidene-D-galactopyranose.

FIG. 36. Oral bioavailability of abuse-resistant hydrocodone glyco-peptide conjugates, measured as free hydrocodone.

FIG. 37. Oral bioavailability of an abuse-resistant hydrocodone amino acid-carbohydrate conjugate, measured as free hydrocodone.

FIG. 38. illustrates nucleosides and conjugation sites.

FIG. 39. Oral bioavailability in rats for hydrocodone vs. EEFFFI-HC at a dose (1 mg/kg) approximating a therapeutic human dose equivalent measured as free hydrocodone.

FIG. 40. Oral bioavailability in rats for hydrocodone vs. EEFFF-HC at a dose (1 mg/kg) approximating a therapeutic human dose equivalent measured as free hydrocodone.

FIG. 41. Oral bioavailability in rats for hydrocodone vs. YYI-HC at a dose (1 mg/kg) approximating a therapeutic human dose equivalent measured as free hydrocodone.

FIG. 42. Oral bioavailability in rats for hydrocodone vs. DDI-HC at a dose (1 mg/kg) approximating a therapeutic human dose equivalent measured as free hydrocodone.

FIG. 43. Oral bioavailability in rats for hydrocodone vs. YYFFI-HC at a dose (1 mg/kg) approximating a therapeutic human dose equivalent measured as free hydrocodone.

FIG. 44. Oral bioavailability in rats for hydrocodone vs. EEFFI-HC at a dose (5 mg/kg) approaching a human overdose equivalent measured as free hydrocodone.

FIG. 45. Oral bioavailability in rats for hydrocodone vs. YYI-HC at a dose (5 mg/kg) approaching a human overdose equivalent measured as free hydrocodone.

FIG. 46. Oral bioavailability in rats for hydrocodone vs. DDI-HC at a dose (5 mg/kg) approaching a human overdose equivalent measured as free hydrocodone.

FIG. 47. Oral bioavailability in rats for hydrocodone vs. YYFFI-HC at a dose (5 mg/kg) approaching a human overdose equivalent measured as free hydrocodone.

FIG. 48. Decrease in bioavailability of EEFFF-HC as compared to hydrocodone by the intranasal route of administration measured as free hydrocodone.

FIG. 49. Decrease in bioavailability of YYI-HC as compared to hydrocodone by the intranasal route of administration measured as free hydrocodone.

FIG. 50. Decrease in bioavailability of DDI-HC as compared to hydrocodone by the intranasal route of administration measured as free hydrocodone.

FIG. 51. Decrease in bioavailability of YYFFI-HC as compared to hydrocodone by the intranasal route of administration measured as free hydrocodone.

FIG. 52. Decrease in bioavailability of EEFFI-HC as compared to hydrocodone by the intravenous route of administration measured as free hydrocodone.

FIG. 53. Decrease in bioavailability of EEFFF-HC as compared to hydrocodone by the intravenous route of administration measured as free hydrocodone.

FIG. 54. Decrease in bioavailability of YYI-HC as compared to hydrocodone by the intravenous route of administration measured as free hydrocodone.

FIG. 55. Decrease in bioavailability of YYFFI-HC as compared to hydrocodone by the intravenous route of administration measured as free hydrocodone.

FIG. 56. Oral bioavailability of hydrocodone plus hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 57. Oral bioavailability of hydrocodone (concentration vs. time) following administration of hydrocodone bitratrate or YYFFI-HC at 1 mg/kg (equimolar doses with equivalent content of hydrocodone base) in rats, measured as free hydrocodone.

FIG. 58. Oral bioavailability of hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 59. Oral bioavailability of hydrocodone plus hydromorphone (concentration vs. time) following administration of hydrocodone bitratrate or YYFFI-HC at 2 mg/kg (equimolar doses with equivalent content of hydrocodone base) in rats, measured as free hydrocodone.

FIG. 60. Oral bioavailability of hydrocodone (concentration vs. time) following administration of hydrocodone bitratrate or YYFFI-HC at 2 mg/kg (equimolar doses with equivalent content of hydrocodone base) in rats, measured as free hydrocodone.

FIG. 61. Oral bioavailability of hydromorphone (concentration vs. time) following administration of hydrocodone bitratrate or YYFFI-HC at 2 mg/kg (equimolar doses with equivalent content of hydrocodone base) in rats, measured as free hydrocodone.

FIG. 62. Oral bioavailability of hydrocodone plus hydromorphone (concentration vs. time) following administration of hydrocodone bitratrate or YYFFI-HC at 5 mg/kg (equimolar doses with equivalent content of hydrocodone base) in rats, measured as free hydrocodone.

FIG. 63. Oral bioavailability of hydrocodone (concentration vs. time) following administration of hydrocodone bitratrate or YYFFI-HC at 5 mg/kg (equimolar doses with equivalent content of hydrocodone base) in rats, measured as free hydrocodone.

FIG. 64. Oral bioavailability of hydromorphone (concentration vs. time) following administration of hydrocodone bitratrate or YYFFI-HC at 5 mg/kg (equimolar doses with equivalent content of hydrocodone base) in rats, measured as free hydrocodone.

FIG. 65. Oral bioavailability of hydrocodone plus hydromorphone (concentration vs. time) following administration of hydrocodone bitratrate or YYFFI-HC at 25 mg/kg (equimolar doses with equivalent content of hydrocodone base) in rats, measured as free hydrocodone.

FIG. 66. Oral bioavailability of hydrocodone (concentration vs. time) following administration of hydrocodone bitratrate or YYFFI-HC at 25 mg/kg (equimolar doses with equivalent content of hydrocodone base) in rats, measured as free hydrocodone.

FIG. 67. Oral bioavailability of hydromorphone (concentration vs. time) following administration of hydrocodone bitratrate or YYFFI-HC at 25 mg/kg (equimolar doses with equivalent content of hydrocodone base) in rats, measured as free hydrocodone.

FIG. 68. Oral bioavailability (AUC₀₋₄ₕ) of hydrocodone plus hydromorphone (concentration vs. dose) in proportion to dose following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 69. Oral bioavailability (AUC₀₋₄ₕ) of hydrocodone plus hydromorphone in proportion to human equivalent doses (HED) following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 70. Oral bioavailability (Cₘₐₓ) of hydrocodone plus hydromorphone (concentration vs. dose) in proportion to dose following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 71. Oral bioavailability (Cₘₐₓ) of hydrocodone plus hydromorphone in proportion to human equivalent doses (HED) following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 72. Intravenous bioavailability of hydrocodone plus hydromorphone and YYFFI-HC (concentration vs. time) following administration of hydrocodone bitratrate or YYFFI-HC at 1 mg/kg (equimolar doses with equivalent content of hydrocodone base) in rats, measured as free hydrocodone.

FIG. 73. Intravenous bioavailability of hydrocodone (concentration vs. time) following administration of hydrocodone bitratrate or YYFFI-HC at 1 mg/kg (equimolar doses with equivalent content of hydrocodone base) in rats, measured as free hydrocodone.

FIG. 74. Intravenous bioavailability of hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 75. Intranasal bioavailability of hydrocodone plus hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 76. Intranasal bioavailability of hydrocodone (concentration vs. time) following administration of hydrocodone bitratrate or YYFFI-HC at 1 mg/kg (equimolar doses with equivalent content of hydrocodone base) in rats, measured as free hydrocodone.

FIG. 77. Intranasal bioavailability of hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 78. Oral bioavailability of hydrocodone plus hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 79. Oral bioavailability of hydrocodone (concentration vs. time) following administration of hydrocodone bitratrate or YYFFI-HC at 1 mg/kg (equimolar doses with equivalent content of hydrocodone base) in rats, measured as free hydrocodone.

FIG. 80. Oral bioavailability of hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 81. Oral bioavailability of hydrocodone plus hydromorphone (concentration vs. time) following administration of hydrocodone bitratrate or YYFFI-HC at 2 mg/kg (equimolar doses with equivalent content of hydrocodone base) in rats, measured as free hydrocodone.

FIG. 82. Oral bioavailability of hydrocodone (concentration vs. time) following administration of hydrocodone bitratrate or YYFFI-HC at 2 mg/kg (equimolar doses with equivalent content of hydrocodone base) in rats, measured as free hydrocodone.

FIG. 83. Oral bioavailability of hydromorphone (concentration vs. time) following administration of hydrocodone bitratrate or YYFFI-HC at 2 mg/kg (equimolar doses with equivalent content of hydrocodone base) in rats, measured as free hydrocodone.

FIG. 84. Oral bioavailability of hydrocodone plus hydromorphone (concentration vs. time) following administration of hydrocodone bitratrate or YYFFI-HC at 5 mg/kg (equimolar doses with equivalent content of hydrocodone base) in rats, measured as free hydrocodone.

FIG. 85. Oral bioavailability of hydrocodone (concentration vs. time) following administration of hydrocodone bitratrate or YYFFI-HC at 5 mg/kg (equimolar doses with equivalent content of hydrocodone base) in rats, measured as free hydrocodone.

FIG. 86. Oral bioavailability of hydromorphone (concentration vs. time) following administration of hydrocodone bitratrate or YYFFI-HC at 5 mg/kg (equimolar doses with equivalent content of hydrocodone base) in rats, measured as free hydrocodone.

FIG. 87. Oral bioavailability of hydrocodone plus hydromorphone (concentration vs. time) following administration of hydrocodone bitratrate or YYFFI-HC at 25 mg/kg (equimolar doses with equivalent content of hydrocodone base) in rats, measured as free hydrocodone.

FIG. 88. Oral bioavailability of hydrocodone (concentration vs. time) following administration of hydrocodone bitratrate or YYFFI-HC at 25 mg/kg (equimolar doses with equivalent content of hydrocodone base) in rats, measured as free hydrocodone.

FIG. 89. Oral bioavailability of hydromorphone (concentration vs. time) following administration of hydrocodone bitratrate or YYFFI-HC at 25 mg/kg (equimolar doses with equivalent content of hydrocodone base) in rats, measured as free hydrocodone.

FIG. 90. Oral bioavailability (AUC₀₋₄) of hydrocodone plus hydromorphone (concentration vs. dose) in proportion to dose following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 91. Oral bioavailability (AUC₀₋₄) of hydrocodone plus hydromorphone in proportion to human equivalent doses (HED) following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 92. Oral bioavailability (Cₘₐₓ) of hydrocodone plus hydromorphone (concentration vs. dose) in proportion to dose following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 93. Oral bioavailability (Cₘₐₓ) of hydrocodone plus hydromorphone in proportion to human equivalent doses (HED) following administration of hydrocodone bitratrate or YYFFI-HC at escalating doses (1, 2, 5, and 25 mg/kg-equimolar doses with equivalent content of hydrocodone base) in rats, measured as free hydrocodone.

FIG. 94. Intravenous bioavailability of hydrocodone plus hydromorphone and YYFFI-HC (concentration vs. time) following administration of hydrocodone bitratrate or YYFFI-HC at 1 mg/kg (equimolar doses with equivalent content of hydrocodone base) in rats, measured as free hydrocodone.

FIG. 95. Intravenous bioavailability of hydrocodone (concentration vs. time) following administration of hydrocodone bitratrate or YYFFI-HC at 1 mg/kg (equimolar doses with equivalent content of hydrocodone base) in rats, measured as free hydrocodone.

FIG. 96. Intravenous bioavailability of hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 97. Intranasal bioavailability of hydrocodone plus hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 98. Intranasal bioavailability of hydrocodone (concentration vs. time) following administration of hydrocodone bitratrate or YYFFI-HC at 1 mg/kg (equimolar doses with equivalent content of hydrocodone base) in rats, measured as free hydrocodone.

FIG. 99. Intranasal bioavailability of hydromorphone (concentration vs. time) following administration of hydrocodone bitratrate or YYFFI-HC at 1 mg/kg (equimolar doses with equivalent content of hydrocodone base) in rats, measured as free hydrocodone.

FIG. 100. depicts oxycodone.

FIG. 101. depicts oxycodone with lysine branched peptides.

FIG. 102. depicts a glycosylated oxycodone.

FIG. 103. depicts formation of an enol ether with serine.

FIG. 104. depicts niacin and biotin.

FIG. 105. Oral bioavailability of abuse-resistant oxycodone disubstituted tripeptide conjugates, measured as free oxycodone.

FIG. 106. Oral bioavailability of abuse-resistant oxycodone disubstituted tripeptide conjugates, measured as free oxycodone.

FIG. 107. Oral bioavailability of abuse-resistant oxycodone disubstituted tripeptide conjugates, measured as free oxycodone.

FIG. 108. Oral bioavailability of abuse-resistant oxycodone disubstituted tripeptide conjugates, measured as free oxycodone.

FIG. 109. Oral bioavailability of abuse-resistant oxycodone disubstituted tripeptide conjugates, measured as free oxycodone.

FIG. 110. Oral bioavailability of abuse-resistant oxycodone disubstituted tripeptide conjugates, measured as free oxycodone.

FIG. 111. Oral bioavailability of abuse-resistant oxycodone disubstituted tripeptide conjugates, measured as free oxycodone.

FIG. 112. Oral bioavailability of abuse-resistant oxycodone disubstituted tripeptide conjugates, measured as free oxycodone.

FIG. 113. Oral bioavailability of abuse-resistant oxycodone disubstituted tripeptide conjugates, measured as free oxycodone.

FIG. 114. Oral bioavailability of abuse-resistant oxycodone disubstituted tripeptide conjugates, measured as free oxycodone.

FIG. 115. Oral bioavailability of abuse-resistant oxycodone disubstituted tripeptide conjugates, measured as free oxycodone.

FIG. 116. Oral bioavailability of abuse-resistant oxycodone disubstituted tripeptide conjugates, measured as free oxycodone.

FIG. 117. Oral bioavailability of abuse-resistant oxycodone disubstituted tripeptide conjugates, measured as free oxycodone.

FIG. 118. Oral bioavailability of abuse-resistant oxycodone disubstituted tripeptide conjugates, measured as free oxycodone.

FIG. 119. Oral bioavailability of abuse-resistant oxycodone disubstituted tripeptide conjugates, measured as free oxycodone.

FIG. 120. Oral bioavailability of abuse-resistant oxycodone disubstituted tripeptide conjugates, measured as free oxycodone.

FIG. 121. Oral bioavailability of abuse-resistant oxycodone disubstituted tripeptide conjugates, measured as free oxycodone.

FIG. 122. Oral bioavailability of abuse-resistant oxycodone disubstituted tripeptide conjugates, measured as free oxycodone.

FIG. 123. Oral bioavailability of abuse-resistant oxycodone disubstituted tripeptide conjugates, measured as free oxycodone.

FIG. 124. Intranasal bioavailability of abuse-resistant oxycodone disubstituted tripeptide conjugates, measured as free oxycodone.

FIG. 125. Intranasal bioavailability of abuse-resistant oxycodone disubstituted tripeptide conjugates, measured as free oxycodone.

FIG. 126. Intranasal bioavailability of abuse-resistant oxycodone disubstituted tripeptide conjugates, measured as free oxycodone.

FIG. 127. Intravenous bioavailability of abuse-resistant oxycodone disubstituted tripeptide conjugates, measured as free oxycodone.

FIG. 128. Intranasal bioavailability of abuse-resistant oxycodone disubstituted tripeptide conjugates, measured as free oxycodone.

FIG. 129. Intranasal bioavailability of abuse-resistant oxycodone disubstituted tripeptide conjugates, measured as free oxycodone.

FIG. 130. Intranasal bioavailability of abuse-resistant oxycodone disubstituted tripeptide conjugates, measured as free oxycodone.

FIG. 131. Intranasal bioavailability of abuse-resistant oxycodone disubstituted tripeptide conjugates, measured as free oxycodone.

FIG. 132. Intranasal bioavailability of abuse-resistant oxycodone disubstituted tripeptide conjugates, measured as free oxycodone.

FIG. 133. Intranasal bioavailability of abuse-resistant oxycodone disubstituted tripeptide conjugates, measured as free oxycodone.

FIG. 134. Intranasal bioavailability of abuse-resistant oxycodone disubstituted tripeptide conjugates, measured as free oxycodone.

FIG. 135. Intranasal bioavailability of abuse-resistant oxycodone disubstituted tripeptide conjugates, measured as free oxycodone.

FIG. 136. Intranasal bioavailability of abuse-resistant oxycodone disubstituted tripeptide conjugates, measured as free oxycodone.

FIG. 137. Intranasal bioavailability of abuse-resistant oxycodone disubstituted tripeptide conjugates, measured as free oxycodone.

FIG. 138. Intranasal bioavailability of abuse-resistant oxycodone disubstituted tripeptide conjugates, measured as free oxycodone.

FIG. 139. Intranasal bioavailability of abuse-resistant oxycodone disubstituted tripeptide conjugates, measured as free oxycodone.

FIG. 140. Intranasal bioavailability of abuse-resistant oxycodone disubstituted tripeptide conjugates, measured as free oxycodone.

FIG. 141. Intranasal bioavailability of abuse-resistant oxycodone disubstituted tripeptide conjugates, measured as free oxycodone.

FIG. 142. Oral bioavailability in rats of oxycodone vs. P2L₍₂₎-Oxycodone at a dose (2.5 mg/kg) approximating a therapeutic human dose equivalent measured as free oxycodone.

FIG. 143. Decrease in bioavailability of P2L₍₂₎-Oxycodone as compared to oxycodone by the intranasal route of administration-dose 2.5 mg/kg measured as free oxycodone.

FIG. 144. Decrease in bioavailability of P2L₍₂₎-Oxycodone as compared to oxycodone by the intravenous route of administration-dose 0.5 mg/kg measured as free oxycodone.

### DETAILED DESCRIPTION OF THE INVENTION

The invention relates to changing the pharmacokinetic and pharmacological properties of active agents through covalent modification. Covalent attachment of a chemical moiety to an active agent can change the rate and extent of absorption, metabolism, distribution, and elimination of the active agent. When administered at a normal therapeutic dose the bioavailablility (area under the time-versus-concentration curve; AUC) of the active agent is similar to that of the parent active agent compound. As the oral dose is increased, however, the bioavailability of the covalently modified active agent relative to the parent active agent begins to decline. At suprapharmacological doses the bioavailability of the active agent conjugate is substantially decreased as compared to the parent active agent. The relative decrease in bioavailability at higher doses abates the euphoria obtained when doses of the active agent conjugate are taken above those of the intended prescription. This in turn diminishes the abuse potential, whether unintended or intentionally sought.

Persons that abuse prescription drugs commonly seek to increase their euphoria by snorting or injecting the drugs. These routes of administration increase the rate and extent of drug absorption and provide a faster, nearly instantaneous, effect. This increases the amount of drug that reaches the central nervous system where it has its effect. In a particular embodiment of the invention the bioavailability of the covalently modified active agent is substantially decreased by the intranasal and intravenous routes as compared to the parent active agent. Thus the illicit practice of snorting and shooting the drug loses its advantage.

In accordance with the present invention and as used herein, the following terms are defined with the following meanings, unless explicitly stated otherwise. For additional methods of attaching active agents to carriers, see application number U.S. Ser. No. 10/156,527, and/or PCT/US03/05524, and/or PCT/US03/05525 and/or PCT/US04/17204 each of which is hereby incorporated by reference in its entirety.

The invention utilizes covalent modification of an active agent to decrease its potential for causing overdose or being abused. The active agent is covalently modified in a manner that decreases its pharmacological activity, as compared to the unmodified active agent, at doses above those considered therapeutic, e.g., at doses inconsistent with the manufacturer's instructions. When given at lower doses, such as those intended for therapy, the covalently modified active agent retains pharmacological activity similar to that of the unmodified active agent. The covalent modification of the active agent may comprise the attachment of any chemical moiety through conventional chemistry.

Compounds, compositions and methods of the invention provide reduced potential for overdose, reduced potential for abuse or addiction and/or improve the active agent's characteristics with regard to high toxicities or suboptimal release profiles. Without wishing to be limited to the below theory, we believe that in some instances overdose protection results from a natural gating mechanism at the site of hydrolysis that limits the release of the active agent from the prodrug at greater than therapeutically prescribed amounts. Therefore, abuse resistance is provided by limiting the “rush” or “high” available from the active agent released by the prodrug and limiting the effectiveness of alternative routes of administration.

Throughout this application the use of “opioid” is meant to include any drug that activates the opioid receptors found in the brain, spinal cord and gut. There are three broad classes of opioids: naturally occurring opium alkaloids, such as morphine (the prototypical opioid) and codeine; semi-synthetics such as heroine, oxycodone and hydrocodone that are produced by modifying natural opium alkaloids and have similar chemical structures; and pure synthetics such as fentanyl and methadone that are not produced from opium and may have very different chemical structures than the opium alkaloids. Other opioids include dihydromorphine, ethylmorphine, methyldihydromorphinone, hydromorphone, hydroxymorphone, oxymorphone, naltrexone, methadone, levorphanol, dihydrocodeine, meperidine, diphenoxylate, sufentanil, alfentanil, propoxyphene, pentazocine, nalbuphine, butorphanol, buprenorphine, meptazinol, dezocine, and pharmaceutically acceptable salts thereof.

Throughout this application the use of “oxyocodone” is meant to include a narcotic alkaloid (chemical formula C₁₈H₂₁NO₄) and its derivatives such as the hydrochloride salt of oxycodone. Oxycodone is related to codeine and is used as an analgesic and/or a sedative. Oxycodone is a powerful and potentially addictive opioid analgesic synthesized from thebaine. It is similar to codeine, but is more potent and has a higher dependence potential. It is effective orally and is often marketed in combination with aspirin (Percodan®) or acetaminophen (Percocet®) for the relief of pain. It is also sold in a sustained-release form under the trade name Oxycontin®. All of these deriviatives or combinations of oxycodone are encompassed by the present invention.

Throughout this application the use of “hydrocodone” is meant to include a semisynthetic narcotic analgesic and antitussive prepared from codeine with multiple actions qualitatively similar to those of codeine. It is commonly used for the relief of moderate to moderately severe pain. Trade names include Anexsia®, Hycodan®, Hycomine®, Lorcet®, Lortab®, Norco®, Tussionex®, Tylox®, and Vicodin®. Derivatives of hydrocodone, such as hydrocodone bitartrate and hydrocodone polistirex, are encompassed by the present invention.

Throughout this application the use of “peptide” is meant to include a single amino acid, a dipeptide, a tripeptide, an oligopeptide, a polypeptide, or the carrier peptide. Oligopeptide is meant to include from 2 amino acids to 70 amino acids. Further, at times the invention is described as being an active agent attached to an amino acid, a dipeptide, a tripeptide, an oligopeptide, or polypeptide to illustrate specific embodiments for the active agent conjugate. Preferred lengths of the conjugates and other preferred embodiments are described herein.

Throughout this application the use of “chemical moiety” is meant to include at least amino acids, peptides, glycopeptides, carbohydrates, lipids, nucleosides, or vitamins.

Throughout this application, the term “increment” is used to define a numerical value in varying degrees of precision, e.g., to the nearest 10, 1, 0.1, 0.01, etc. The increment can be rounded to any measurable degree of precision. For example, the range 1 to 100 or increments therein includes ranges such as 20 to 80, 5 to 50, 0.4 to 98, and 0.04 to 98.05.

Use of phrases such as “decreased”, “reduced”, “diminished”, or “lowered” includes at least a 10% change in pharmacological activity with greater percentage changes being preferred for reduction in abuse potential and overdose potential. For instance, the change may also be greater than 25%, 35%, 45%, 55%, 65%, 75%, 85%, 95%, 96%, 97%, 98%, 99%, or other increments greater than 10%.

Use of the phrase “similar pharmacological activity” means that two compounds exhibit curves that have substantially the same AUC, Cmax, Tmax, Cmin, and/or t1/2 parameters, preferably within about 30% of each other, more preferably within about 25%, 20%, 10%, 5%, 2%, 1%, or other increments less than 30%.

“Carbohydrates” includes sugars, starches, cellulose, and related compounds. e.g., (CH₂O)ₙ, wherein n is an integer larger than 2 or C(H₂O)ₙ₋₁, with n larger than 5. More specific examples include for instance, fructose, glucose, lactose, maltose, sucrose, glyceraldehyde, dihydroxyacetone, erythrose, ribose, ribulose, xylulose, galactose, mannose, sedoheptulose, neuraminic acid, dextrin, and glycogen.

A “glycoprotein” is a compound containing carbohydrate (or glycan) covalently linked to protein. The carbohydrate may be in the form of a monosaccharide, disaccharide(s). oligosaccharide(s), polysaccharide(s), or their derivatives (e.g. sulfo- or phospho-substituted).

A “glycopeptide” is a compound consisting of carbohydrate linked to an oligopeptide composed of L- and/or D-amino acids. A glyco-amino-acid is a saccharide attached to a single amino acid by any kind of covalent bond. A glycosyl-amino-acid is a compound consisting of saccharide linked through a glycosyl linkage (O—, N— or S—) to an amino acid.

A “composition” as used herein, refers broadly to any composition containing a described molecule conjugates. The composition may comprise a dry formulation, an aqueous solution, or a sterile composition. Compositions comprising the molecules described herein may be stored in freeze-dried form and may be associated with a stabilizing agent such as a carbohydrate. In use, the composition may be deployed in an aqueous solution containing salts, e.g., NaCl, detergents, e.g., sodium dodecyl sulfate (SDS), and other components.

A “controlled substance” is a substance subject to federal regulation of its manufacture, sale, or distribution because of the potential for, or proved evidence of, abuse; because of its potential for psychic or physiological dependence; because it constitutes a public health risk; because of the scientific evidence of its pharmacologic effect; or because of its role as a precursor of other controlled substances.

Important note regarding stereochemistry: This patent is meant to cover all compounds discussed regardless of absolute configurations. Thus, natural, L-amino acids are discussed but the use of D-amino acids are also included.

The following abbreviations may be in this application:
BOC=t-butyloxycarbonyl
CMC=carboxymethylcellulose
DIPEA=di-isopropyl ethyl amine
mp=melting point
NMR=nuclear magnetic resonance
   OSu=hydroxysuccinimido ester
   Nia=Niacin
   Bio=Biotin

The attached chemical moiety may be any chemical substance that decreases the pharmacological activity until the active agent is released. Preferably the chemical moiety is a single amino acid, dipeptide or tripeptide, tetrapeptide, pentapeptide, or hexapeptide. The active agent binds to specific sites to produce various effects (Hoebel, et al., 1989). The attachment of certain chemical moieties can therefore diminish or prevent binding to these biological target sites. Preferably, absorption of the composition into the brain is prevented or substantially diminished and/or delayed when delivered by routes other than oral administration.

The attached chemical moiety may further comprise naturally occurring or synthetic substances. This would include but is not limited to the attachment of an active agent to one or more amino acids, peptides, lipids, carbohydrates, glycopeptides, nucleic acids or vitamins. These chemical moieties could be expected to affect delayed release in the gastrointestinal tract and prevent rapid onset of the desired activity, particularly when delivered by parenteral routes. (Hoebel, B. G., L. Hernandez, et al. (1989). “Microdialysis studies of brain norepinephrine, serotonin, and dopamine release during ingestive behavior. Theoretical and clinical implications.” Ann N Y Acad Sci 575: 171-91).

For each of the embodiments recited herein, the amino acid or peptide may comprise of one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. In another embodiment the amino acid or peptide is comprised of one or more of the naturally occurring (D) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. In another embodiment the amino acid or peptide is comprised of one or more unnatural, non-standard or synthetic amino acids such as, aminohexanoic acid, biphenylalanine, cyclohexylalanine, cyclohexylglycine, diethylglycine, dipropylglycine, 2,3-diaminoproprionic acid, homophenylalanine, homoserine, homotyrosine, naphthylalanine, norleucine, ornithine, pheylalanine(4-fluoro), phenylalanine(2,3,4,5,6 pentafluoro), phenylalanine(4-nitro), phenylglycine, pipecolic acid, sarcosine, tetrahydroisoquinoline-3-carboxylic acid, and tert-leucine. In another embodiment the amino acid or peptide comprises of one or more amino acid alcohols. In another embodiment the amino acid or peptide comprises of one or more N-methyl amino acids.

In another embodiment, the specific carriers are utilized as a base short chain amino acid sequence and additional amino acids are added to the terminus or side chain. In another embodiment, the above amino acid sequence may have one more of the amino acids substituted with one of the 20 naturally occurring amino acids. It is preferred that the substitution be with an amino acid which is similar in structure or charge compared to the amino acid in the sequence. For instance, isoleucine (Ile)[I] is structurally very similar to leucine (Leu)[L], whereas, tyrosine (Tyr)[Y] is similar to phenylalanine (Phe)[F], whereas serine (Ser)[S] is similar to threonine (Thr)[T], whereas cysteine (Cys)[C] is similar to methionine (Met)[M], whereas alanine (Ala)[A] is similar to valine (Val)[V], whereas lysine (Lys)[K] is similar to arginine (Arg)[R], whereas asparagine (Asn)[N] is similar to glutamine (Gln)[Q], whereas aspartic acid (Asp)[D] is similar to glutamic acid (Glu)[E], whereas histidine (His)[H] is similar to proline (Pro)[P], and glycine (Gly)[G] is similar to tryptophan (Trp)[W]. In the alternative the preferred amino acid substitutions may be selected according to hydrophilic properties (i.e. polarity) or other common characteristics associated with the 20 essential amino acids. While preferred embodiments utilize the 20 natural amino acids for their GRAS characteristics, it is recognized that minor substitutions along the amino acid chain which do not effect the essential characteristics of the amino are also contemplated.

In one embodiment the carrier range is between one to 12 chemical moieties with one to 8 moieties being preferred. In another embodiment the number of chemical moieties attached is selected from 1, 2, 3, 4, 5, 6, or 7, etc. In another embodiment of the invention the molecular weight of the carrier portion of the conjugate is below about 2,500, more preferably below about 1,000 and most preferably below about 500.

The compositions and methods of the invention may be applied to various therapeutically valuable active agents (e.g., drugs) and include, for example, stimulants such as anticonvulsants, muscle relaxants, antidepressants, anxiolytics, benzodiazepines, sedatives, hypnotics, narcotics, steroids, respiratory agents, including antihistamines, antipsychotics including risperidone, and nonsteroidal anti-inflammatory agents.

Exemplary narcotics include opioids, hydrocodone, oxycodone, morphine, dihydromorphine, ethylmorphine, codeine, hydromorphone, hydroxymorphone, oxymorphone, methyldihydromorphinone, methadone, fentanyl, levorphanol, dihydrocodeine, meperidine, diphenoxylate, sufentanil, alfentanil, propoxyphene, pentazocine, nalbuphine, butorphanol, buprenorphine, meptazinol, naltrexone, dezocine or pharmaceutically acceptable salts thereof.

The compositions and methods of the invention provide active agents which when bound to the chemical moiety provide safer and/or more effective dosages for the above recited active agent classes through improved bioavailability curves and/or safer Cₘₐₓ and/or reduce area under the curve for bioavailability, particularly for abused substances taken in doses above therapeutic levels. As a result, the compositions and methods of the invention may provide improved methods of treatment for attention deficit hyperactivity, attention deficit hyperactivity disorder (ADHD), attention deficit disorder (ADD), cognitive decline associated with acquired immunodeficiency syndrome (AIDS) or AIDS-related complex, depression, anxiety and anxiety related disorders, psychosis, nicotine addiction, narcotic addiction, alcoholism, narcolepsy, and/or analgesia.

In one embodiment the chemical moiety is comprised of an amino acid or a polypeptide. Preferred amino acid and peptide chemical moieties include, for example, Lys, Ser, Ala, Phe, Ile, Pro-Pro-Leu, Pro-Pro-Ile, Val-Val, Lys-Lys, Gly-Gly-Ile, Phe-Phe-Ile, Phe-Phe-Leu, Thr-Thr-Val, Tyr-Tyr-Val, Tyr-Tyr-Phe, Glu-Glu-Val, Asp-Asp-Val, Lys-Lys-Val, Glu-Glu-Phe-Phe-Ile, Glu-Glu-Phe-Phe-Phe, Tyr-Tyr-Ile, Asp-Asp-Ile, Tyr-Tyr-Phe-Phe-Ile, Tyr-Tyr-Lys-Tyr-Tyr, Phe-Phe-Lys-Phe-Phe, Glu-Glu-Phe-Phe-Ile, (Lys-Lys-Gly-Gly)₂, and [(1)-Lys-(d)-Lys-Leu]₂. In some embodiments, the active agent is disubstituted with one or more of the preceding chemical moieties.

Another embodiment of the invention is a composition for preventing overdose comprising an active agent which has been covalently bound to a chemical moiety.

Another embodiment of the invention is a composition for safely delivering an active agent comprising providing a therapeutically effective amount of said active agent which has been covalently bound to a chemical moiety wherein said chemical moiety reduces the rate of absorption of the active agent as compared to delivering the unbound active agent.

Another embodiment of the invention is a composition for reducing drug toxicity comprising providing a patient with an active agent which has been covalently bound to a chemical moiety wherein said chemical moiety increases the rate of clearance of an active agent when given at doses exceeding those within the therapeutic range of said active agent.

Another embodiment of the invention is a composition for reducing drug toxicity comprising providing a patient with an active agent which has been covalently bound to a chemical moiety wherein said chemical moiety provides a serum release curve which does not increase above said active agent toxicity level when given at doses exceeding those within the therapeutic range of said active agent.

Another embodiment of the invention is a composition for reducing bioavailability of active agent comprising active agent covalently bound to a chemical moiety wherein said bound active agent maintains a steady-state serum release curve which provides a therapeutically effective bioavailability but prevents spiking or increase blood serum concentrations compared to unbound active agent when given at doses exceeding those within the therapeutic range of said active agent.

Another embodiment of the invention is a composition for preventing a Cₘₐₓ spike for active agent while still providing a therapeutically effective bioavailability curve comprising an active agent that has been covalently bound to a chemical moiety.

Another embodiment of the invention is a composition for preventing a toxic release profile in a patient comprising active agent covalently bound to a chemical moiety wherein said bound active agent maintains a steady-state serum release curve which provides a therapeutically effective bioavailability but prevents spiking or increase blood serum concentrations compared to unbound active agent.

In one embodiment, the opioid prodrug (a compound of one of the formulas described herein) may exhibit one or more of the following advantages over free opioids. The opioid prodrug may prevent overdose by exhibiting a reduced pharmacological activity when administered at higher than therapeutic doses, e.g., higher than the prescribed dose. Yet when the opioid prodrug is administered at therapeutic doses, the opioid prodrug may retain similar pharmacological activity to that achieved by administering unbound opioid, e.g., currently marketed opioid compositions. Also, the opioid prodrug may prevent abuse by exhibiting stability under conditions likely to be employed by illicit chemists attempting to release the opioid. The opioid prodrug compositions may prevent abuse by exhibiting reduced bioavailability when it is administered via parenteral routes, particularly the intravenous (“shooting”), intranasal (“snorting”), and/or inhalation (“smoking”) routes that are often employed in illicit use. Thus, the opioid prodrug may reduce the euphoric effect associated with opioid abuse. Thus, the opioid prodrug may prevent and/or reduce the potential of abuse and/or overdose when the opioid prodrug is used in a manner inconsistent with the manufacturer's instructions, e.g., consuming the opioid prodrug at a higher than therapeutic dose or via a non-oral route of administration.

Preferably, the opioid prodrug exhibits an unbound opioid oral bioavailability of at least about 60% AUC (area under the curve), more preferably at least about 70%, 80%, 90%, 95%, 96%, 97%, 98%, 99%, or other increments greater than 60%. Preferably, the opioid prodrug exhibits an unbound opioid parenteral, e.g., intranasal, bioavailability of less than about 70% AUC, more preferably less than about 50%, 30%, 20%, 15%, 10%, 5%, 4%, 3%, 2%, 1%, or other increments less than 70%. For certain treatments, it is desirable that the opioid prodrug exhibits both the oral and parenteral bioavailability characteristics described above.

In another embodiment, the toxicity of the opioid prodrug is substantially lower than that of the unbound opioid. For example, in a preferred embodiment, the acute toxicity is 1-fold, 2-fold, 3-fold, 4-fold, 5-fold, 6-fold, 7-fold, 8-fold, 9-fold, 10-fold less, or increments therein less lethal than oral administration of unbound opioid.

Preferably, the opioid prodrug provides a serum release curve that does not increase above opioid's toxicity level when administered at higher than therapeutic doses. The opioid prodrug may exhibit a reduced rate of opioid absorption and/or an increased rate of clearance compared to the free opioid. The opioid prodrug may also exhibit a steady-state serum release curve. Preferably, the opioid prodrug provides bioavailability but prevents Cmax spiking or increased blood serum concentrations. Pharmacokinetic parameters are described in the Examples. For example, the pharmacological parameters (AUC, Cmax, Tmax, Cmin, and/or t1/2) are preferably within 80% to 125%, 80% to 120%, 85% to 125%, 90% to 110%, or increments therein, of the given values. It should be recognized that the ranges can, but need not be symmetrical, e.g., 85% to 105%.

The opioid prodrug may exhibit delayed and/or sustained release characteristics. Delayed release prevents rapid onset of pharmacological effects, and sustained release is a desirable feature for particular dosing regimens, e.g., once a day regimens. The opioid prodrug may achieve the release profile independently. Alternatively, the opioid prodrug may be pharmaceutically formulated to enhance or achieve such a release profile. It may be desirable to reduce the amount of time until onset of pharmacological effect, e.g., by formulation with an immediate release product.

Accordingly, the invention also provides methods comprising providing, administering, prescribing, or consuming an opioid prodrug. The invention also provides pharmaceutical compositions comprising an opioid prodrug. The formulation of such a pharmaceutical composition can optionally enhance or achieve the desired release profile.

In one embodiment, the invention provides methods for treating a patient comprising administering a therapeutically effective amount of an opioid prodrug, i.e., an amount sufficient to prevent, ameliorate, and/or eliminate the symptoms of a disease. These methods can be used to treat any disease that may benefit from opioid-type drugs including, but not limited to pain management.

The methods of treatment include combination therapies that further comprise administering one or more therapeutic agents in addition to administering an opioid prodrug. The active ingredients can be formulated into a single dosage form, or they can be formulated together or separately among multiple dosage forms. The active ingredients can be administered simultaneously or sequentially in any order.

The dose range of the opioid prodrug for humans will depend on a number of factors including the age, weight, and condition of the patient. Tablets and other dosage forms provided in discrete units can contain a daily dose, or an appropriate fraction thereof, of one or more opioid prodrugs. The dosage form can contain a dose of about 2.5 mg to about 500 mg, about 10 mg to about 250 mg, about 10 mg to about 100 mg, about 25 mg to about 75 mg, or increments therein of one or more of the opioid prodrugs. In a preferred embodiment, the dosage form contains 30 mg, 50 mg, or 70 mg of an opioid prodrug.

The dosage form can utilize any one or any combination of known release profiles including, but not limited to immediate release, extended release, pulse release, variable release, controlled release, timed release, sustained release, delayed release, and long acting.

Another embodiment of the invention is a compound of Formula I:
A-Xₙ-Zₘ
wherein A is active agent as defined herein; X is a chemical moiety as defined herein and n is between 1 and 50 and increments thereof; and Z is a further chemical moiety different from X which acts as an adjuvant and m is between 1 and 50 and increments thereof. In another embodiment n is between 1 and 10 and m is 0. It should be recognized that the compounds of this formula may be used alone or in combination with any of the recited embodiments of the invention.

Embodiments of the invention provide compositions which allow the active agent to be therapeutically effective when delivered at the proper dosage but reduces the rate of absorption or extent of bioavailability of the active agent when given at doses exceeding those within the therapeutic range of the active agent. Embodiments of the invention also provide compositions wherein the covalently bound chemical moiety increases the rate of clearance of active agent when given at doses exceeding those within the therapeutic range of the active agent.

In another embodiment the compositions have substantially lower toxicity compared to unbound active agent. In another embodiment the compositions reduce or eliminate the possibility of overdose by oral administration. In another embodiment the compositions reduce or eliminate the possibility of overdose by intranasal administration. In another embodiment the compositions reduce or eliminate the possibility of overdose by injection.

In another embodiment, the conjugates of the invention may further comprise a polymer blend which comprises at least one hydrophilic polymer and at least one water-insoluble polymer. The polymer may be used according to industry standard to further enhance the sustained release properties of the active agent conjugate without reducing the abuse resistance. Hydrophilic polymers suitable for use in the sustained release formulation include: one or more natural or partially or totally synthetic hydrophilic gums such as acacia, gum tragacanth, locust bean gum, guar gum, or karaya gum, modified cellulosic substances such as methylcellulose, hydroxomethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art or a combination of such polymers.

These hydrophilic polymers gel and would dissolve slowly in aqueous acidic media thereby allowing the active agent conjugate to diffuse from the gel in the stomach. When the gel reaches the intestines it would dissolve in controlled quantities in the higher pH medium to allow sustained release. Preferred hydrophilic polymers are the hydroxypropyl methylcelluloses such as those manufactured by The Dow Chemical Company and known as Methocel ethers, such as Methocel E10M.

Other formulations may further comprise pharmaceutical additives including, but not limited to: lubricants such as magnesium stearate, calcium stearate, zinc stearate, powdered stearic acid, hydrogenated vegetable oils, talc, polyethylene glycol, and mineral oil; colorants; binders such as sucrose, lactose, gelatin, starch paste, acacia, tragacanth, povidone polyethylene glycol, Pullulan and corn syrup; glidants such as colloidal silicon dioxide and talc; surface active agents such as sodium lauryl sulfate, dioctyl sodium sulfosuccinate, triethanolamine, polyoxyethylene sorbitan, poloxalkol, and quarternary ammonium salts; preservatives and stabilizers; excipients such as lactose, mannitol, glucose, fructose, xylose, galactose, sucrose, maltose, xylitol, sorbitol, chloride, sulfate and phosphate salts of potassium, sodium, and magnesium; and/or any other pharmaceutical additives known to those of skill in the art. Colorants include, but are not limited to, Emerald Green Lake, FD&C Red No. 40, FD&C Yellow No. 6, D&C Yellow No. 10, or FD&C Blue No. 1 and other various certified color additives (See 21 CFR, Part 74). In one preferred embodiment, a sustained release formulation further comprises magnesium stearate and Emerald Green Lake.

An active agent conjugate, which is further formulated with excipients may be manufactured according to any appropriate method known to those of skill in the art of pharmaceutical manufacture. For instance, the active agent conjugate and a hydrophilic polymer may be mixed in a mixer with an aliquot of water to form a wet granulation. The granulation may be dried to obtain hydrophilic polymer encapsulated granules of active agent-conjugate. The resulting granulation may be milled, screened, then blended with various pharmaceutical additives, water insoluble polymer, and additional hydrophilic polymer. The formulation may then tableted and may further be film coated with a protective coating which rapidly dissolves or disperses in gastric juices.

However, it should be noted that the active agent conjugate controls the release of active agent into the digestive tract over an extended period of time resulting in an improved profile when compared to immediate release combinations and reduces and/or prevents abuse without the addition of the above additives. In a preferred embodiment no further sustained release additives are required to achieve a blunted or reduced pharmacokinetic curve (e.g. reduced euphoric effect) while achieving therapeutically effective amounts of active agent release.

The compounds of the invention can be administered by a variety of dosage forms. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, health bars, confections, animal feeds, cereals, yogurts, cereal coatings, foods, nutritive foods, functional foods and combinations thereof.

However, the most effective means for delivering the abuse-resistant compounds of the invention is orally, to permit maximum release of the active agent to provide therapeutic effectiveness and/or sustained release while maintaining abuse resistance. When delivered by the oral route the active agent is released into circulation, preferably over an extended period of time as compared to active agent alone.

Formulations of the invention suitable for oral administration can be presented as discrete units, such as capsules, caplets or tablets. These oral formulations also can comprise a solution or a suspension in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which is then placed in the feeding tube of a patient who is unable to swallow.

Soft gel or soft gelatin capsules may be prepared, for example by dispersing the formulation in an appropriate vehicle (vegetable oils are commonly used) to form a high viscosity mixture. This mixture is then encapsulated with a gelatin based film using technology and machinery known to those in the soft gel industry. The industrial units so formed are then dried to constant weight.

Chewable tablets, for example may be prepared by mixing the formulations with excipients designed to form a relatively soft, flavored, tablet dosage form that is intended to be chewed rather than swallowed. Conventional tablet machinery and procedures, that is both direct compression and granulation, i.e., or slugging, before compression, can be utilized. Those individuals involved in pharmaceutical solid dosage form production are versed in the processes and the machinery used as the chewable dosage form is a very common dosage form in the pharmaceutical industry.

Film coated tablets, for example may be prepared by coating tablets using techniques such as rotating pan coating methods or air suspension methods to deposit a contiguous film layer on a tablet.

Compressed tablets, for example may be prepared by mixing the formulation with excipients intended to add binding qualities to disintegration qualities. The mixture is either directly compressed or granulated then compressed using methods and machinery known to those in the industry. The resultant compressed tablet dosage units are then packaged according to market need, i.e., unit dose, rolls, bulk bottles, blister packs, etc.

The invention also contemplates the use of biologically-acceptable carriers which may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

Preferred plasticizers may be selected from the group consisting of diethyl phthalate, diethyl sebacate, triethyl citrate, cronotic acid, propylene glycol, butyl phthalate, dibutyl sebacate, castor oil and mixtures thereof, without limitation. As is evident, the plasticizers may be hydrophobic as well as hydrophilic in nature. Water-insoluble hydrophobic substances, such as diethyl phthalate, diethyl sebacate and castor oil are used to delay the release of water-soluble vitamins, such as vitamin B6 and vitamin C. In contrast, hydrophilic plasticizers are used when water-insoluble vitamins are employed which aid in dissolving the encapsulated film, making channels in the surface, which aid in nutritional composition release.

It should be understood that in addition to the ingredients particularly mentioned above, the formulations of this invention can include other suitable agents such as flavoring agents, preservatives and antioxidants. Such antioxidants would be food acceptable and could include vitamin E, carotene, BHT or other antioxidants known to those of skill in the art.

Other compounds which may be included by admixture are, for example, medically inert ingredients, e.g. solid and liquid diluent, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Liquid dispersions for oral administration may be syrups, emulsions or suspensions. The syrups may contain as carrier, for example, saccharose or saccharose with glycerol and/or mannitol and/or sorbitol. In particular a syrup for diabetic patients can contain as carriers only products, for example sorbitol, which do not metabolize to glucose or which metabolize only a very small amount to glucose. The suspensions and the emulsions may contain a carrier, for example a natural gum, agar, sodium alginate, pectin, methylcellulose, carboxymethylcellulose or polyvinyl alcohol.

The dose range for adult human beings will depend on a number of factors including the age, weight and condition of the patient and the administration route. Tablets and other forms of presentation provided in discrete units conveniently contain a daily dose, or an appropriate fraction thereof, of one of the present compounds. For example, units may contain from 5 mg to 500 mg, but more usually from 10 mg to 250 mg, of one of the present compounds.

It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art.

The pharmaceutical compositions of the invention can be administered in a partial, i.e., fractional dose, one or more times during a 24 hour period. Fractional, single, double, or other multiple doses can be taken simultaneously or at different times during a 24 hour period. The doses can be uneven doses with regard to one another or with regard to the individual components at different administration times. Preferably, a single dose is administered once daily. The dose can be administered in a fed or fasted state.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

It will be appreciated that the pharmacological activity of the compositions of the invention can be demonstrated using standard pharmacological models that are known in the art. Furthermore, it will be appreciated that the inventive compositions can be incorporated or encapsulated in a suitable polymer matrix or membrane for site-specific delivery, or can be functionalized with specific targeting agents capable of effecting site specific delivery. These techniques, as well as other drug delivery techniques are well known in the art.

In another embodiment of the invention, the solubility and dissolution rate of the composition is substantially changed under physiological conditions encountered in the intestine, at mucosal surfaces, or in the bloodstream. In another embodiment the solubility and dissolution rate substantially decrease the bioavailability of the said pharmaceutical, particularly at doses above those intended for therapy. In another embodiment the decrease in bioavailability occurs upon oral administration. In another embodiment the decrease in bioavailability occurs upon intranasal administration. In another embodiment the decrease in bioavailability occurs upon intravenous administration.

Another particular embodiment of the invention provides that when the covalently modified active agent is provided for oral dosing in the form (e.g., a tablet or capsule) it is resistant to manipulation. Crushing of the tablet or disruption of the capsule does not substantially increase the rate and amount of active agent absorbed when compositions of the invention are ingested.

For each of the described embodiments one or more of the following characteristics may be realized. The toxicity of the compound is substantially lower than that of the unbound active agent. The covalently bound chemical moiety reduces or eliminates the possibility of overdose by oral administration. The covalently bound chemical moiety reduces or eliminates the possibility of overdose by intranasal administration. The covalently bound chemical moiety reduces or eliminates the possibility of overdose by injection.

The invention further provides methods for altering active agent in a manner that decreases their potential for abuse. Methods of the invention provide various ways to regulate pharmaceutical dosage through covalent attachment of active agent to different chemical moieties. One embodiment provides a method of preventing overdose comprising administering to an individual an active agent which has been covalently bound to a chemical moiety.

Another embodiment provides a method of safely delivering an active agent comprising providing a therapeutically effective amount of an active agent which has been covalently bound to a chemical moiety wherein the chemical moiety reduces the rate of absorption of active agent as compared to delivering the unbound active agent.

Another embodiment provides a method of reducing drug toxicity comprising providing a patient with an active agent which has been covalently bound to a chemical moiety wherein the chemical moiety increases the rate of clearance of a pharmacologically active active agent when given at doses exceeding those within the therapeutic range of active agent.

Another embodiment provides a method of reducing drug toxicity comprising providing a patient with an active agent which has been covalently bound to a chemical moiety wherein the chemical moiety provides a serum release curve which does not increase above the active agent's toxicity level when given at doses exceeding those within the therapeutic range for the unbound active agent.

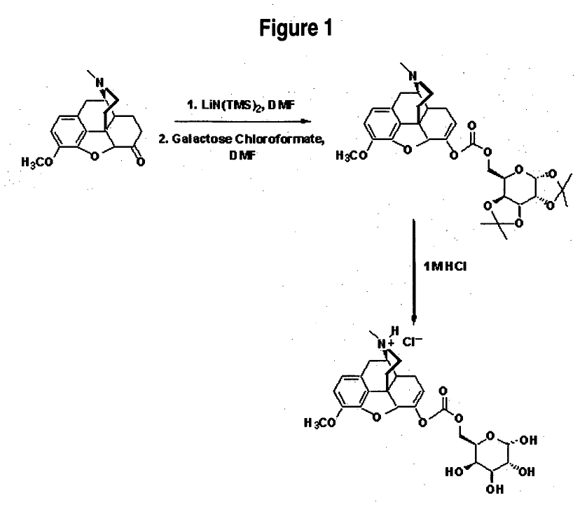

Another embodiment provides a method of reducing bioavailability of an active agent comprising providing active agent covalently bound to a chemical moiety wherein the bound active agent maintains a steady-state serum release curve which provides a therapeutically effective bioavailability but prevents spiking or increase blood serum concentrations compared to unbound active agent when given at doses exceeding those within the therapeutic range for the unbound active agent. Another embodiment provides a method of preventing a Cₘₐₓ spike for active agent while still providing a therapeutically effective bioavailability curve comprising providing an active agent which has been covalently bound to a chemical moiety. In another embodiment, methods of the invention provide bioavailability curves similar to those found in FIGS. 1-195.

Another embodiment provides a method for preventing a toxic release profile in a patient comprising administering to a patient an active agent covalently bound to a chemical moiety wherein said bound active agent maintains a steady-state serum release curve which provides a therapeutically effective bioavailability but prevents spiking or increase blood serum concentrations compared to unbound active agent.

Another embodiment of the invention is a method for reducing or preventing abuse of a pharmaceutical composition, comprising providing, administering, or prescribing said composition to a human in need thereof, wherein said composition comprises a chemical moiety covalently attached to an active agent such that the pharmacological activity of active agent is substantially decreased when the composition is used in a manner inconsistent with the manufacturer's instructions. Another embodiment of the invention is a method for reducing or preventing abuse of a pharmaceutical composition, comprising consuming said composition, wherein said composition comprises a chemical moiety covalently attached to an active agent such that the pharmacological activity of the active agent is substantially decreased when the composition is used in a manner inconsistent with the manufacturer's instructions.

Another embodiment of the invention is a method of preventing overdose of a pharmaceutical composition, comprising providing, administering, or prescribing said pharmaceutical composition to a human in need thereof, wherein said composition comprises a chemical moiety covalently attached to an active agent in a manner that substantially decreases the potential of overdose from active agent. Another embodiment of the invention is a method of preventing overdose of a pharmaceutical composition, comprising consuming said pharmaceutical composition, wherein said composition comprises a chemical moiety covalently attached to active agent in a manner that substantially decreases the potential of overdose from the active agent.

Another embodiment of the invention is a method for reducing or preventing the euphoric effect of a pharmaceutical composition, comprising providing, administering, or prescribing said composition to a human in need thereof, wherein said composition comprises a chemical moiety covalently attached to an active agent such that the pharmacological activity of active agent is substantially decreased when the composition is used in a manner inconsistent with the manufacturer's instructions. Another embodiment of the invention is a method for reducing or preventing the euphoric effect of a pharmaceutical composition, comprising consuming said composition, wherein said composition comprises a chemical moiety covalently attached to an active agent such that the pharmacological activity of active agent is substantially decreased when the composition is used in a manner inconsistent with the manufacturer's instructions.

Another embodiment of the invention is any of the preceding methods wherein said pharmaceutical composition is adapted for oral administration, and wherein said active agent is resistant to release from said chemical moiety when the composition is administered parenterally, such as intranasally or intravenously. Preferably, said active agent may be released from said chemical moiety in the presence of acid and/or enzymes present in the stomach, intestinal tract, or blood serum. Optionally, said composition may be in the form of a tablet, capsule, oral solution, or oral suspension.

Another embodiment of the invention is any of the preceding methods wherein said chemical moiety is an amino acid, oligopeptide, polypeptide, carbohydrate, glycopeptide, nucleic acid, or vitamin. Preferably, said chemical moiety is an amino acid, oligopeptide, or polypeptide. Where the chemical moiety is a polypeptide, preferably said polypeptide comprises fewer than 70 amino acids, fewer than 50 amino acids, fewer than 10 amino acids, or fewer than 6 amino acids.

Another embodiment of the invention is any of the preceding methods wherein said covalent attachment comprises an ester or carbonate bond. Another embodiment of the invention is any of the preceding methods wherein said active agent covalently attaches to a chemical moiety through a ketone and/or hydroxyl in a pharmaceutically acceptable oral dosage form.

Another embodiment of the invention is any of the preceding methods wherein said composition yields a therapeutic effect without substantial euphoria. Preferably, said active agent provides a therapeutically bioequivalent AUC when compared to active agent alone but does provide a Cₘₐₓ which results in euphoria.

Another embodiment of the invention is a method for reducing or preventing abuse of a pharmaceutical composition, comprising orally administering said composition to a human in need thereof, wherein said composition comprises an amino acid or peptide covalently attached to active agent such that the pharmacological activity of active agent is substantially decreased when the composition is used in a manner inconsistent with the manufacturer's instructions.

Another embodiment is a method of preventing overdose of a pharmaceutical composition, comprising orally administering said pharmaceutical composition to a human in need thereof, wherein said composition comprises an amino acid or peptide covalently attached to active agent in a manner that substantially decreases the potential of active agent to result in overdose.

Another embodiment is a method for reducing or preventing the euphoric effect of a pharmaceutical composition, comprising orally administering said composition to a human in need thereof, wherein said composition comprises an amino acid or peptide covalently attached to active agent such that the pharmacological activity of active agent is substantially decreased when the composition is used in a manner inconsistent with the manufacturer's instructions.

For each of the recited methods of the invention the following properties may be achieved through bonding active agent to the chemical moiety. In one embodiment, the toxicity of the compound may be substantially lower than that of the active agent when delivered in its unbound state or as a salt thereof. In another embodiment, the possibility of overdose by oral administration is reduced or eliminated. In another embodiment, the possibility of overdose by intranasal administration is reduced or eliminated. In another embodiment, the possibility of overdose by injection administration is reduced or eliminated.

Another embodiment of the invention provides methods of treating various diseases or conditions comprising administering compounds or compositions of the invention which further comprise commonly prescribed active agents for the respective illness or diseases wherein the active agent is covalently attached to a chemical moiety.

Another embodiment of the invention provides a method of treating cognitive decline associated with acquired immunodeficiency syndrome (AIDS) or AIDS-related complex comprising administering to a patient compounds or compositions of the invention.

Another embodiment of the invention provides a method of treating depression comprising administering to a patient compounds or compositions of the invention. Another embodiment of the invention provides a method of treating anxiety and anxiety related disorders comprising administering to a patient compounds or compositions of the invention. Another embodiment of the invention provides a method of treating psychosis comprising administering to a patient compounds or compositions of the invention.

Another embodiment of the invention provides a method of treating nicotine addiction comprising administering to a patient compounds or compositions of the invention. Another embodiment of the invention provides a method of treating narcotic addiction comprising administering to a patient compounds or compositions of the invention. Another embodiment of the invention provides a method of treating alcoholism comprising administering to a patient compounds or compositions of the invention.

Another embodiment of the invention provides a method of treating narcolepsy comprising administering to a patient compounds or compositions of the invention. Another embodiment of the invention provides a method of providing analgesia comprising administering to a patient compounds or compositions of the invention.

In order to facilitate a more complete understanding of the invention, Examples are provided below. However, the scope of the invention is not limited to specific embodiments disclosed in these Examples, which are for purposes of illustration only.

### EXAMPLES

The invention is illustrated by pharmacokinetic studies with hydrocodone and oxycodone that have been covalently modified by attachment to various moieties such as an individual amino acid, specific short chained amino acid sequences such as di-, tri-, and pentapeptides, or carbohydrates such as ribose, etc. Studies include pharmacokinetic evaluations of the various drug conjugates administered by the oral, intranasal, and intravenous routes. Collectively the compounds demonstrate that active agents may be modified by covalent attachment to various moieties and retain their therapeutic value at normal doses while preventing potential overdose by oral administration and prevention of abuse through intranasal and intravenous administration.

Carrier Bound Compounds

### Examples 1 through 51

### Hydrocodone

Applicability of Abuse Resistance for the Narcotic Analgesics Demonstrated Through the Use of Hydrocodone.

Examples 1 through 51 illustrate the applicability of a number of peptide-active agent compositions in reducing the potential for overdose while maintaining their therapeutic value wherein the peptides are conjugated to the active agent hydrocodone (HC). Exemplary compounds which were substituted at the 6 position of hydrocodone are termed EEFFI-HC, EEFFF-HC, YYI-HC, DDI-HC, and YYFFI-HC.

Oral, intranasal, and intravenous bioavailability studies of hydrocodone and hydrocodone conjugates were conducted in male Sprague-Dawley rats. Doses of hydrocodone bitartrate and hydrocodone conjugates containing equivalent amounts of hydrocodone were administered in deionized water. Oral administration was in 0.5 ml by gavage needle (with the exception of YYI-HC, which was delivered as a solid in gelatin capsules). Intranasal doses were administered by placing 20 microliters into the nasal flares of rats anesthetized with isoflurane. Intravenous administration was in 0.1 ml by tail vein injection. Plasma was collected by retroorbital sinus puncture under isoflurane anesthesia. Hydrocodone and hydromorphone (major active metabolite) concentrations were determined by LC/MS/MS.

The below examples are illustrative only and the below amino acid sequences attached to hydrocodone is not meant to be limiting. As such, synthesis and attachment of hydrocodone may be accomplished for instance view the following exemplary methods.

Hydrocodone Synthetic Examples Carbohydrates

### Example 1

### Galacto-Hydrocodone

FIG. 1 illustrates preparation of Galacto-Hydrocodone.

| Reagents | MW | Weight | | mmoles | Molar Equivalents |
|---|---|---|---|---|---|
| 1. Hydrocodone | 299 | 0.223 | g | 0.75 | 1.0 |
| 1. LiN(TMS)₂ in THF | 1M | 1.13 | ml | 1.13 | 1.5 |
| 1. DMF | — | 5 | ml | — | — |
| 2. Galactose | — | — | | 1.49 | 2.0 |
| Chloroformate | | | | | |
| 2. DMF | — | 3 | ml | — | — |
| 3. 1M HCl | 1M | 30 | ml | — | — |
| 3. Acetone | — | 20 | ml | — | — |

Galacto-Hydrocodone

To a solution of hydrocodone in DMF was added LiN(TMS)₂ in THF via syringe. The solution was stirred at ambient temperatures for 5 minutes then the chloroformate of galactose in DMF was added via syringe. The resulting solution was stirred at ambient temperatures for 2 hours. A TLC was taken (9:1 CHCl₃:MeOH; UV and 5% H₂SO₄ in MeOH; R_{f(product)}=˜0.5). Reaction was neutralized to pH 7 with 6M HCl. Solvent was removed. Final product was purified using preparative TLC (0-10% MeOH in CHCl₃). Solid was collected as a white powder (0.180 g, 41% yield): ¹H NMR (DMSO-d₆) δ 1.28 (2s, 6H), 1.37 (s, 3H), 1.44 (3, 3H), 1.49 (m, 2H), 1.88 (dt, 1H), 2.08 (m, 2H), 2.29 (s, 4H), 2.40 (m, 2H), 2.90 (d, 1H), 3.09 (s, 1H), 3.73 (s, 3H), 3.99 (dd, 1H), 4.14 (t, 1H), 4.26 (dt, 2H), 4.39 (d, 1H), 4.63 (d, 1H), 4.95 (s, 1H), 5.48 (d, 1H), 5.68 (d, 1H), 6.65 (d, 1H), 6.74 (d, 1H); MS Calculated mass=585.6 Found=586.4 (M+H).

To the protected galactose intermediate was added 30 ml of 1M HCl and 20 ml acetone. The resulting solution was stirred at ambient temperatures for 3 hours. Solvent was removed and final product dried under vacuum. Solid was collected as a white solid: MS Calculated mass=505.5 Found=506.4 (M+H).

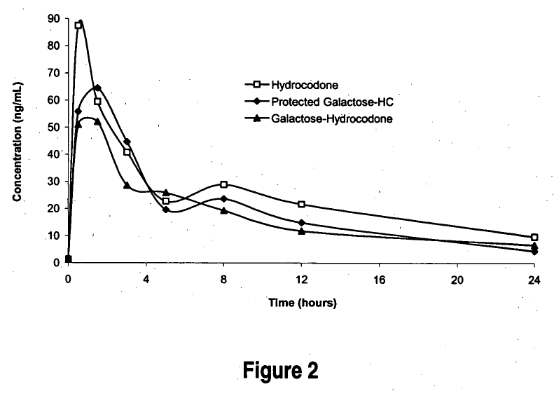

FIG. 2 depicts oral bioavailability of abuse-resistant hydrocodone carbohydrate conjugates, measured as free hydrocodone (with measured plasma levels by ELISA).

### Example 2

### Ribo-Hydrocodone

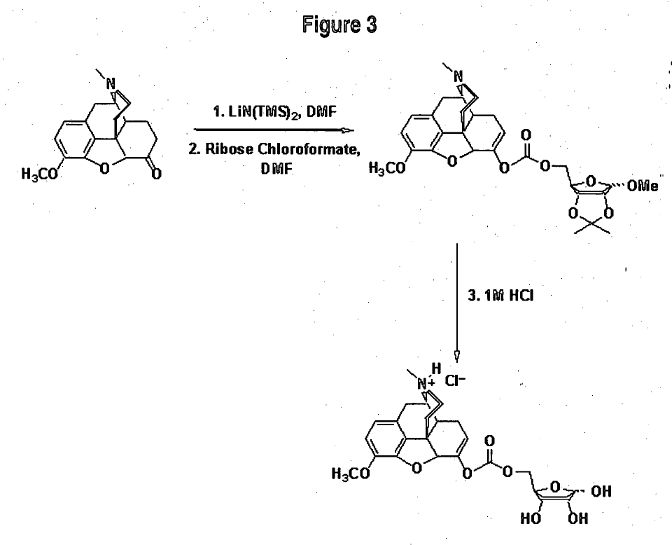

FIG. 3 illustrates preparation of Ribo-Hydrocodone.

| Reagents | MW | Weight | | mmoles | Molar Equivalents |
|---|---|---|---|---|---|
| 1. Hydrocodone | 299 | 0.733 | g | 2.45 | 1.0 |
| 1. LiN(TMS)₂ in THF | 1M | 3.68 | ml | 3.68 | 1.5 |
| 1. DMF | — | 8 | ml | — | — |
| 2. Ribose | — | — | | 4.90 | 2.0 |
| Chloroformate | | | | | |
| 2. DMF | — | 3 | ml | — | — |
| 3. 1M HCl | 1M | 10 | ml | — | — |

Ribo-Hydrocodone

To a solution of hydrocodone in DMF was added LiN(TMS)₂ in THF via syringe. The solution was stirred at ambient temperatures for 5 minutes then the chloroformate of ribose in DMF was added via syringe. The resulting solution was stirred at ambient temperatures for 2 hours. A TLC was taken (9:1 CHCl₃:MeOH; UV and 5% H₂SO₄ in MeOH; R_{f(product)}=˜0.5). Reaction was neutralized to pH 7 with 1M HCl. Solvent was removed. Crude product was taken up in CHCl₃ (50 ml), washed with water (3×50 ml), dried over MgSO₄, filtered and solvent removed. Final product was purified using preparative HPLC (10 mM CH₃COONH₄/MeCN; 0-20 min: 80/20→0/100). Solid was collected as a clear, colorless glass (0.095 g, 7% yield): ¹H NMR (DMSO-d₆) δ 1.26 (s, 3H), 1.39 (s, 3H), 1.50 (m, 2H), 1.89 (s, 4H), 2.08 (m, 2H), 2.29 (s, 4H), 2.40 (m, 2H), 2.88 (d, 1H), 3.08 (m, 1H), 3.25 (s, 3H), 3.73 (s, 3H), 4.12 (m, 2H), 4.28 (t, 1H), 4.58 (d, 1H), 4.72 (d, 1H), 4.97 (s, 1H), 4.98 (s, 1H), 5.70 (s, 1H), 6.66 (d, 1H), 6.75 (d, 1H). MS Calculated mass=529.2 Found=530.4 (M+H).

To the protected ribose intermediate was added 10 ml of 1M HCl. The resulting solution was stirred at ambient temperatures for 2 hours. Solvent was removed and final product dried under vacuum. Solid was collected as a waxy, slightly yellow solid (0.092 g, quant.): ¹H NMR (DMSO-d₆) δ 1.51 (t, 1H), 1.83 (d, 1H), 2.41 (dt, 1H), 2.27 (t, 1H), 2.63 (dd, 1H), 2.80 (s, 3H), 2.96 (m, 2H), 3.20 (m, 1H), 3.75 (s, 3H), 3.82-4.34 (br m, 12H), 5.15 (s, 1H), 5.72 (s, 1H), 6.75 (d, 1H), 6.88 (d, 1H), 11.37 (br s, 1H).

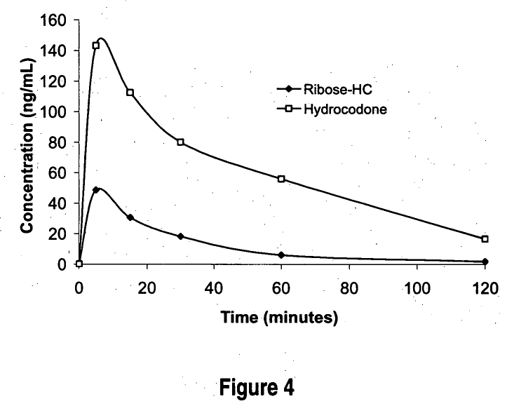

FIG. 4 illustrates intranasal bioavailability of abuse-resistant hydrocodone carbohydrate conjugate, measured as free hydrocodone (with measured plasma levels by ELISA).

Single Amino Acids

### Example 3

### Leu-Hydrocodone

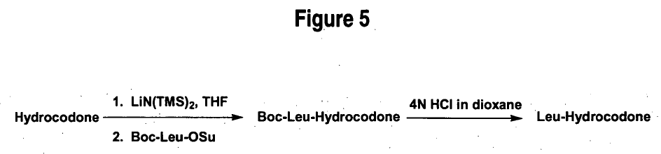

FIG. 5 illustrates preparation of Leu-Hydrocodone.

| Reagents | MW | Weight | | mmoles | Molar Equivalents |
|---|---|---|---|---|---|
| 1. Hydrocodone | 299 | 1.00 | g | 3.34 | 1.0 |
| 1. LiN(TMS)₂ in THF | 1M | 10.5 | ml | 10.5 | 3.15 |
| 1. THF | — | 25 | ml | — | — |
| 2. Boc-Leu-OSu | 328 | 3.28 | g | 10.0 | 3.0 |

Leu-Hydrocodone

To a solution of hydrocodone in THF was added LiN(TMS)₂ in THF via syringe. The solution was stirred at ambient temperatures for 5 minutes then Boc-Leu-OSu was added. The resulting reaction mixture was stirred at ambient temperatures for 18 hours. Reaction was neutralized to pH 7 with 6M HCl. Solvent was removed. Crude material was taken up in CHCl₃ (100 ml), washed with sat. NaHCO₃ (3×100 ml), dried over MgSO₄, filtered, and solvent removed. Solid was collected as a yellow powder (1.98 g, 95% yield): ¹H NMR (DMSO-d₆) δ 0.86 (dd, 6H), 1.31 (s, 9H), 1.46 (s, 2H), 1.55 (m, 2H), 1.69 (m, 1H), 1.87 (dt, 1H), 2.07 (dt, 2H), 2.29 (s, 3H), 2.43 (m, 2H), 2.93 (d, 1H), 3.11 (s, 1H), 3.72 (s, 3H), 3.88 (dt, 1H), 4.03 (dt, 1H), 4.87 (s, 1H), 5.51 (d, 1H), 6.65 (d, 1H), 6.73 (d, 11H), 6.90 (s, 1H).

To the Boc-Leu-Hydrocodone was added 25 ml of 4N HCl in dioxane. The resulting mixture was stirred at ambient temperatures for 18 hours. Solvent was removed and final product dried under vacuum. Solid was collected as a slightly yellow solid (1.96 g, 97% yield): ¹H NMR (DMSO-d₆) δ 0.94 (d, 6H), 1.52 (m, 1H), 1.75-1.90 (m, 4H), 2.22 (dt, 1H), 2.34 (dt, 1H), 2.64 (q, 1H), 2.75 (s, 3H), 2.95-3.23 (m, 4H), 3.74 (s, 3H), 3.91 (d, 1H), 4.07 (s, 1H), 5.10 (s, 1H), 5.72 (d, 1H), 6.76 (d, 1H), 6.86 (d, 1H), 8.73 br s, 3H).

### Example 4

### Glu-Hydrocodone

Synthesis of Glu-Hydrocodone

Glu-Hydrocodone was prepared by a similar method to Example 3 except the amino acid starting material was Boc-Glu(OtBu)-OSu.

### Example 5

### Ile-Hydrocodone

Synthesis of Ile-Hydrocodone

Ile-Hydrocodone was prepared by a similar method to Example 3 except the amino acid starting material was Boc-Ile-OSu.

Dipeptides

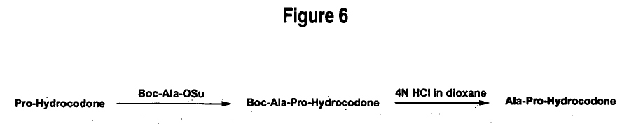

FIG. 6 illustrates preparation of Ala-Pro-Hydrocodone.

### Example 6

### Ala-Pro-Hydrocodone

| Reagents | MW | Weight | | mmoles | Molar Equivalents |
|---|---|---|---|---|---|
| Pro-Hydrocodone | 468 | 0.25 | g | 0.53 | 1.0 |
| Boc-Ala-OSu | 286 | 0.33 | g | 1.2 | 2.26 |
| NMM | 101 | 0.50 | ml | 5.38 | 10.2 |
| DMF | — | 10 | ml | — | — |

Ala-Pro-Hydrocodone

To a solution of Pro-Hydrocodone in DMF was added NMM followed by Boc-Ala-OSu. The solution was stirred at ambient temperatures for 18 hours. Solvent was removed. Crude material was purified using preparative HPLC (Phenomenex Luna C18, 30×250 mm, 5 μM, 100 Å; Gradient: 100 water/0 0.1% TFA-MeCN→0/100; 30 ml/min.). Solid was collected as a slightly yellow powder (0.307 g, 85% yield): ¹H NMR (DMSO-d₆) δ 1.16 (d, 3H), 1.35 (s, 9H), 1.51 (m, 2H), 1.86-2.10 (m, 6H), 2.50 (m, 1H), 2.54 (m, 1H), 2.69 (m, 1H), 2.88 (s, 3H), 3.02 (dd, 1H), 3.26 (d, 1H), 3.55 (m, 1H), 3.67 (m, 1H), 3.72 (s, 3H), 3.80 (s, 1H), 4.25 (m, 1H), 4.43 (d, 1H), 5.01 (s, 1H), 5.59 (d, 1H), 6.75 (d, 1H), 6.88 (d, 1H), 6.99 (t, 1H), 9.91 (br s, 1H).

To the Boc-Ala-Pro-Hydrocodone (0.100 g) was added 10 ml of 4N HCl in dioxane. The resulting mixture was stirred at ambient temperatures for 18 hours. Solvent was removed and final product dried under vacuum. Solid was collected as a slightly yellow solid (0.56 g, 71% yield): ¹H NMR (DMSO-d₆) δ 1.38 (s, 3H), 1.48 (t, 1H), 1.80-2.29 (m, 8H), 2.65 (m, 1H), 2.80 (s, 3H), 2.96 (m, 3H), 3.23 (m, 2H), 3.76 (s, 3H), 3.92 (s, 1H), 4.22 (s, 1H), 4.53 (s, 1H), 5.00 (s, 1H), 5.84 (d, 1H), 6.77 (d, 1H), 6.86 (d, 1H), 8.25 (br s, 3H).

### Example 7

### Glu-Glu-Hydrocodone

Synthesis of Glu-Glu-Hydrocodone

Glu-Glu-Hydrocodone was prepared by a similar method to Example 6 except the amino acid starting material was Boc-Glu(OtBu)-OSu and the conjugate starting material was Glu-Hydrocodone.

### Example 8

### (Pyro)Glu-Glu-Hydrocodone

Synthesis of (Pyro)Glu-Glu-Hydrocodone

The compound (pyro)Glu-Glu-Hydrocodone was prepared by a similar method to Example 6 except the amino acid starting material was Boc-pyroglutamic acid-OSu and the conjugate starting material was Glu-Hydrocodone.

Tripeptides

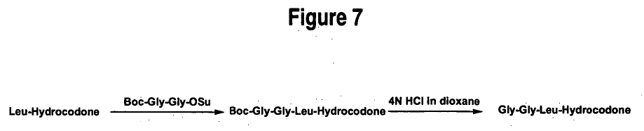

FIG. 7 illustrates the preparation of Gly-Gly-Leu-Hydrocodone.

### Example 9

### Gly-Gly-Leu-Hydrocodone

| Reagents | MW | Weight | | mmoles | Molar Equivalents |
|---|---|---|---|---|---|
| Leu-Hydrocodone | 484 | 2.21 | g | 4.56 | 1.0 |
| Boc-Gly-Gly-OSu | 329 | 3.00 | g | 9.12 | 2.0 |
| NMM | 101 | 5.0 | ml | 45.6 | 10 |
| DMF | — | 100 | ml | — | — |

Gly-Gly-Leu-Hydrocodone

To a solution of Leu-Hydrocodone in DMF was added NMM followed by Boc-Gly-Gly-OSu. The solution was stirred at ambient temperatures for 18 hours. Solvent was removed. Crude material was purified using preparative HPLC (Phenomenex Luna C18, 30×250 mm, 5 μM, 100 Å; Gradient: 90 water/10 0.1% TFA-MeCN→0/100; 30 ml/min.). Solid was collected as a slightly yellow powder (2.08 g, 73% yield): ¹H NMR (DMSO-d₆) δ 0.88 (dd, 6H), 1.38 (s, 9H), 1.53-1.72 (m, 5H), 1.89 (d, 1H), 2.15 (m, 1H), 2.67 (m, 2H), 2.94 (s, 3H), 3.05 (m, 2H), 3.25 (m, 2H), 3.56 (d, 3H), 3.76 (s, 6H), 3.98 (s, 1H), 4.35 (q, 1H), 5.04 (s, 1H), 5.59 (d, 1H), 6.77 (d, 1H), 6.85 (d, 1H), 7.04 (t, 1H), 8.01 (t, 1H), 8.30 (d, 1H), 9.99 (br s, 1H).

To the Boc-Gly-Gly-Leu-Hydrocodone (2.08 g) was added 50 ml of 4N HCl in dioxane. The resulting mixture was stirred at ambient temperatures for 18 hours. Solvent was removed and final product dried under vacuum. Solid was collected as a slightly yellow solid (1.72 g, 86% yield): ¹H NMR (DMSO-d₆) δ 0.89 (dd, 6H), 1.50-1.87 (m, 5H), 2.26 (m, 2H), 2.66 (m, 2H), 2.82-2.97 (m, 5H), 3.21 (m, 2H), 3.60 (m, 4H), 3.88 (m, 5H), 4.37 (m, 1H), 5.04 (s, 1H), 5.60 (s, 1H), 6.79 (d, 2H), 8.07 (br s, 3H), 8.54 (br s, 1H), 8.66 (br s, 1H), 11.29 (br s, 1H).

### Example 10

### Glu-Glu-Glu-Hydrocodone

Synthesis of Glu-Glu-Glu-Hydrocodone

Glu-Glu-Glu-Hydrocodone was prepared by a similar method to Example 9 except the amino acid starting material was Boc-Glu(OtBu)-Glu(OtBu)-OSu and the conjugate starting material was Glu-Hydrocodone.

### Example 11

### Pro-Pro-Leu-Hydrocodone

Synthesis of Pro-Pro-Leu-Hydrocodone

Pro-Pro-Leu-Hydrocodone was prepared by a similar method to Example 9 except the amino acid starting material was Boc-Pro-Pro-OSu.

### Example 12

### Leu-Leu-Leu-Hydrocodone

Synthesis of Leu-Leu-Leu-Hydrocodone

Leu-Leu-Leu-Hydrocodone was prepared by a similar method to Example 9 except the amino acid starting material was Boc-Leu-Leu-OSu.

### Example 13

### Pro-Pro-Ile-Hydrocodone

Synthesis of Pro-Pro-Ile-Hydrocodone

Pro-Pro-Ile-Hydrocodone was prepared by a similar method to Example 9 except the amino acid starting material was Boc-Pro-Pro-OSu and the conjugate starting material was Ile-Hydrocodone.

### Example 14

### Leu-Pro-Leu-Hydrocodone

Synthesis of Leu-Pro-Leu-Hydrocodone

Leu-Pro-Leu-Hydrocodone was prepared by similar methods except the amino acid starting material was Boc-Leu-Pro-OSu.

### Example 15

### Lys-Lys-Ile-Hydrocodone

Synthesis of Lys-Lys-Ile-Hydrocodone

Lys-Lys-Ile-Hydrocodone was prepared by similar methods except the amino acid starting material was Boc-Lys(Boc)-Lys(Boc)-OSu and the conjugate starting material was Ile-Hydrocodone.

### Example 16

### Glu-Glu-Ile-Hydrocodone

Synthesis of Glu-Glu-Ile-Hydrocodone

Glu-Glu-Ile-Hydrocodone was prepared by similar methods except the amino acid starting material was Boc-Glu(OtBu)-Glu(OtBu)-OSu and the conjugate starting material was Ile-Hydrocodone.

### Example 17

### Tyr-Tyr-Ile-Hydrocodone

Synthesis of Tyr-Tyr-Ile-Hydrocodone

Tyr-Tyr-Ile-Hydrocodone was prepared by similar methods except the amino acid starting material was Boc-Tyr(tBu)-Tyr(tBu)-OSu and the conjugate starting material was Ile-Hydrocodone.

Pentapeptides

### Example 18

### Gly-Gly-Gly-Gly-Leu-Hydrocodone

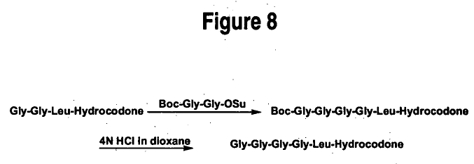

FIG. 8 illustrates preparation of Gly-Gly-Gly-Gly-Leu-Hydrocodone.

| | | | | | Molar |
|---|---|---|---|---|---|
| Reagents | MW | Weight | | mmoles | Equivalents |
| Gly-Gly-Leu-Hydrocodone | 599 | 0.580 | g | 0.970 | 1.0 |
| Boc-Gly-Gly-OSu | 329 | 0.638 | g | 1.94 | 2.0 |
| NMM | 101 | 1.06 | ml | 9.70 | 10 |
| DMF | — | 20 | ml | — | — |

Gly-Gly-Gly-Gly-Leu-Hydrocodone

To a solution of Gly-Gly-Leu-Hydrocodone in DMF was added NMM followed by Boc-Gly-Gly-OSu. The solution was stirred at ambient temperatures for 18 hours. Solvent was removed. Crude material was purified using preparative HPLC (Phenomenex Luna C18, 30×250 mm, 5 μM, 100 Å; Gradient: 85 water/15 0.1% TFA-MeCN→50/50; 30 ml/min.). Solid was collected as a slightly yellow powder (0.304 g, 37% yield).

To the Boc-Gly-Gly-Gly-Gly-Leu-Hydrocodone (0.304 g) was added 25 ml of 4N HCl in dioxane. The resulting mixture was stirred at ambient temperatures for 18 hours. Solvent was removed and final product dried under vacuum. Solid was collected as a slightly yellow solid (0.247 g, 97% yield): ¹H NMR (DMSO-d₆) δ 0.87 (m, 6H), 1.23 (s, 1H), 1.51-1.86 (m, 4H), 2.18 (m, 1H), 2.71 (m, 2H), 2.77 (s, 3H), 2.96 (m, 2H), 3.17 (m, 2H), 3.61 (s, 3H), 3.81-3.84 (m, 10H), 4.22 (m, 1H), 4.36 (m, 1H), 5.09 (m, 1H), 5.59 (d, 1H), 6.74 (dd, 2H), 8.16 (br s, 4H), 8.38 (br s, 1H), 8.74 (br s, 1H), 11.42 (br s, 1H).

### Example 19

### Glu₅-Hydrocodone

Synthesis of Glu₅-Hydrocodone

Glu₅-Hydrocodone was prepared by a similar method to Example 18 except the amino acid starting material was Boc-Glu(OtBu)-Glu(OtBu)-OSu and the conjugate starting material was Glu₃-Hydrocodone.

### Example 20

### Glu₂-Gly₂-Ile-Hydrocodone

Synthesis of Glu₂-Gly₂-Ile-Hydrocodone

Glu₂-Gly₂-Ile-Hydrocodone was prepared by a similar method to Example 18 except the amino acid starting material was Boc-Glu(OtBu)-Glu(OtBu)-OSu and the conjugate starting material was Gly₂-Ile-Hydrocodone.

### Example 21

### Glu₂-Gly₂-Leu-Hydrocodone

Synthesis of Glu₂-Gly₂-Leu-Hydrocodone

Glu₂-Gly₂-Leu-Hydrocodone was prepared by a similar method to Example 18 except the amino acid starting material was Boc-Glu(OtBu)-Glu(OtBu)-OSu and the conjugate starting material was Gly₂-Leu-Hydrocodone.

### Example 22

### Gly₄-Ile-Hydrocodone

Synthesis of Gly₄-Ile-Hydrocodone

Glu₄-Ile-Hydrocodone was prepared by a similar method to Example 18 except the amino acid starting material was Boc-Gly-Gly-OSu and the conjugate starting material was Gly₂-Ile-Hydrocodone.

### Example 23

### Glu₂-Phe₃-Hydrocodone

Synthesis of Glu₂-Phe₃-Hydrocodone

Glu₂-Phe₃-Hydrocodone was prepared by a similar method to Example 18 except the amino acid starting material was Boc-Glu(OtBu)-Glu(OtBu)-OSu and the conjugate starting material was Phe₃-Hydrocodone.

### Example 24

### Lys₂-Gly₂-Ile-Hydrocodone

Synthesis of Lys₂-Gly₂-Ile-Hydrocodone

Lys₂-Gly₂-Ile-Hydrocodone was prepared by a similar method to Example 18 except the amino acid starting material was Boc-Lys(Boc)-Lys(Boc)-OSu and the conjugate starting material was Gly₂-Ile-Hydrocodone.

### Example 25

### Lys₂-Gly₂-Ile-Hydrocodone

Synthesis of Lys₂-Pro₂-Ile-Hydrocodone

Lys₂-Pro₂-Ile-Hydrocodone was prepared by a similar method to Example 18 except the amino acid starting material was Boc-Lys(Boc)-Lys(Boc)-OSu and the conjugate starting material was Pro₂-Ile-Hydrocodone.

### Example 26

### Tyr₂-Gly₂-Ile-Hydrocodone

Synthesis of Tyr₂-Gly₂-Ile-Hydrocodone

Tyr₂-Gly₂-Ile-Hydrocodone was prepared by a similar method to Example 18 except the amino acid starting material was Boc-Tyr(tBu)-Tyr(tBu)-OSu and the conjugate starting material was Gly₂-Ile-Hydrocodone.

### Example 27

### Gly₂-Pro₂-Ile-Hydrocodone

Synthesis of Gly₂-Pro₂-Ile-Hydrocodone

Gly₂-Pro₂-Ile-Hydrocodone was prepared by a similar method to Example 18 except the amino acid starting material was Boc-Gly₂-OSu and the conjugate starting material was Pro₂-Ile-Hydrocodone.

### Example 28

### Asp₂-Phe₂-Ile-Hydrocodone

Synthesis of Asp₂-Phe₂-Ile-Hydrocodone

Asp₂-Phe₂-Ile-Hydrocodone was prepared by a similar method to Example 18 except the amino acid starting material was Boc-Asp(OtBu)-Asp(OtBu)-OSu and the conjugate starting material was Phe₂-Ile-Hydrocodone.

### Example 29

### Glu₂-Asp₂-Ile-Hydrocodone

Synthesis of Glu₂-Asp₂-Ile-Hydrocodone

Glu₂-Asp₂-Ile-Hydrocodone was prepared by a similar method to Example 18 except the amino acid starting material was Boc-Glu(OtBu)-Glu(OtBu)-OSu and the conjugate starting material was Asp₂-Ile-Hydrocodone.

### Example 30

### Lys₂-Asp₂-Ile-Hydrocodone

Synthesis of Lys₂-Asp₂-Ile-Hydrocodone

Lys₂-Asp₂-Ile-Hydrocodone was prepared by a similar method to Example 18 except the amino acid starting material was Boc-Lys(Boc)-Lys(Boc)-OSu and the conjugate starting material was Asp₂-Ile-Hydrocodone.

### Example 31

### Tyr₂-Glu₂-Ile-Hydrocodone

Synthesis of Tyr₂-Glu₂-Ile-Hydrocodone

Tyr₂-Glu₂-Ile-Hydrocodone was prepared by a similar method to Example 18 except the amino acid starting material was Boc-Tyr(tBu)-Tyr(tBu)-OSu and the conjugate starting material was Glu₂-Ile-Hydrocodone.

### Example 32

### Asp₄-Ile-Hydrocodone

Synthesis of Asp₄-Ile-Hydrocodone

Asp₄-Ile-Hydrocodone was prepared by a similar method to Example 18 except the amino acid starting material was Boc-Asp(OtBu)-Asp(OtBu)-OSu and the conjugate starting material was Asp₂-Ile-Hydrocodone.

### Example 33

### Glu₂-Phe₂-Ile-Hydrocodone

Synthesis of Glu₂-Phe₂-Ile-Hydrocodone

Glu₂-Phe₂-Ile-Hydrocodone was prepared by a similar method to Example 18 except the amino acid starting material was Boc-Glu(OtBu)-Glu(OtBu)-OSu and the conjugate starting material was Phe₂-Ile-Hydrocodone.

### Example 34

### Lys₂-Glu₂-Ile-Hydrocodone

Synthesis of Lys₂-Glu₂-Ile-Hydrocodone

Lys₂-Glu₂-Ile-Hydrocodone was prepared by a similar method to Example 18 except the amino acid starting material was Boc-Lys(Boc)-Lys(Boc)-OSu and the conjugate starting material was Glu₂-Ile-Hydrocodone.

### Example 35

### Tyr₂-Phe-Pro-Ile-Hydrocodone

Synthesis of Tyr₂-Phe-Pro-Ile-Hydrocodone

Tyr₂-Phe-Pro-Ile-Hydrocodone was prepared by a similar method to Example 18 except the amino acid starting material was Boc-Tyr(tBu)-Tyr(tBu)-OSu and the conjugate starting material was Phe-Pro-Ile-Hydrocodone.

YYFFI-HC

### Example 36

### Tyr-Tyr-Phe-Phe-Ile-(6-O)-Hydrocodone

Preparation of Tyr-Tyr-Phe-Phe-Ile-(6-O)-hydrocodone

Hydrocodone bitartrate (48.38 g) was stirred in 500 ml 1N NaOH for 5 minutes. Suspension was split into 2 batches and extracted using CHCl₃ (2×250 ml), organics were dried using MgSO₄ and filtered. Solvent was removed and product was obtained as a white powder (29.05 g).

To a solution of hydrocodone freebase (7.12 g) in tetrahydrofuran (THF) (300 ml) was added LiN(TMS)₂ in THF (1M, 36.0 ml) via syringe. The solution was stirred at ambient temperatures for 10 minutes then Boc-Ile-OSu (11.7 g) was added. The resulting reaction mixture was stirred at ambient temperatures for 3 hours. Reaction was neutralized to pH 7 with 1M HCl and stirred for 10 minutes. Solvent was removed. Crude material was taken up in diethyl ether (100 ml), washed with sat. NaHCO₃ (3×100 ml), dried over MgSO₄, filtered, and solvent was removed. Solid was collected as a yellow powder (11.1 g).

To the Boc-Ile-Hydrocodone (11.1 g) was added 125 ml of 4N HCl in dioxane. The resulting mixture was stirred at ambient temperatures for 1 hour. Solvent was removed and final product dried under vacuum. Solid was collected as a slightly yellow powder (10.43 g).

To a suspension of Boc-Phe-Phe-OH (10.0 g) and N-hydroxysuccinimide (NHS) (3.06 g) in acetone (300 ml) was added dicyclohexylcarbodiimide (DCC) (4.99 g). The solution was stirred at ambient temperatures under argon for 18 hrs. Solid dicyclohexylurea (DCU) was filtered away and washed with acetone. Solvent was removed from filtrate. Crude material was recrystallized using a system of acetone and hexane. Solvent was filtered off and the solid was collected as a white powder (12.2 g).

To a solution of Ile-HC.2HCl (6.00 g) in N,N-dimethylformamide (DMF) (150 ml) was added 4-methyl morpholine (NMM) (6.79 ml) followed by Boc-Phe-Phe-OSu (6.93 g). The solution was stirred at ambient temperatures for 18 hours. Solvent was reduced to approximately ¼ total volume, added to sat. NaHCO₃ (˜100 ml), and stirred for 30 minutes. The precipitate was filtered and washed thoroughly with water. Solid material was dried in vacuum, dissolved in a small amount of ethyl acetate, and filtered. Product was obtained as a slightly yellow powder (8.39 g).

To Boc-Phe-Phe-Ile-HC (2.99 g) was added 50 ml 4N HCl in dioxane. The resulting suspension was stirred at ambient temperatures for 1 hour. Solvent was removed and product was dried. Product was obtained as a yellow solid (2.60 g).

To a solution of Boc-Tyr(tBu)-OH (1.00 g) in 15 ml DMF was added O—(N-succinimidyl)-1,1,3,3-tetramethyluronium tetrafluoroborate (TSTU) (0.892 g) and NMM (0.65 ml). After 10 minutes of activation, H-Tyr(tBu)-OH (0.844 g) in 40 ml DMF:dioxane:water (2:2:1) was added. The resulting suspension was stirred at ambient temperature for 4 hours. After this time, water (15 ml) was added and the resulting solution was stirred at ambient temperature for 30 minutes. The solvent volume was reduced to ¼ and extracted with ethyl acetate (250 ml), washed with 5% acetic acid in water (2×150 ml), water (3×150 ml), and brine (150 ml). The organic layer was dried over MgSO₄, filtered, and solvent removed. Crude product was purified using recrystallization with IPAC/hexane solvent system. Final product was isolated as a white solid (1.025 g).

To a suspension of Boc-Tyr(tBu)-Tyr(OtBu)-OH (7.32 g) and NHS (1.54 g) in acetone (150 ml) was added DCC (2.51 g). The solution was stirred at ambient temperatures under argon for 18 hrs. Solid DCU was filtered away and washed with acetone. Solvent was removed from filtrate. Crude material was washed with warm hexane. Solid was collected as a white powder (6.65 g).

To a solution of Phe-Phe-Ile-HC.2HCl (2.63 g) in DMF (100 ml) was added NMM (3.70 ml) followed by Boc-Tyr(tBu)-Tyr(tBu)-OSu (4.41 g). The solution was stirred at ambient temperatures for 18 hours. Solvent was reduced to approximately ¼ total volume, added to sat. NaHCO₃ (˜100 ml), and stirred for 30 minutes. The precipitate was filtered and washed thoroughly with water. Solid material was dried in vacuum and purified by reverse phase HPLC (2.77 g). Product was deprotected using 4N HCl in dioxane (˜50 ml).

To a solution of Phe-Phe-Ile-HC.2HCl (5.00 g) in DMF (250 ml) was added NMM (3.52 ml) followed by Boc-Tyr(tBu)-Tyr(tBu)-OSu (4.61 g). The solution was stirred at ambient temperatures for 6 hours. Solvent was reduced to approximately ¼ total volume, added to sat. NaHCO₃ (˜500 ml), and stirred for 30 minutes. The precipitate was filtered and washed thoroughly with water. Solid material was dried in vacuum overnight, dissolved in methanol, and any remaining solid material was filtered. The solvent was evaporated from the filtrate and the product was recrystallized using ethanol (˜60 ml). The precipitate was filtered and dried in vacuum overnight. Product was collected as a pale brown powder (4.57 g).

Boc-Tyr(OtBu)-Tyr(OtBu)-Phe-Phe-Ile-HC (3.53 g) was deprotected using 4N HCl in dioxane (˜100 ml). This material was stirred at ambient temperatures for ˜1 hour. The solvent was evaporated and the product was collected as a slightly yellow powder (3.64 g).

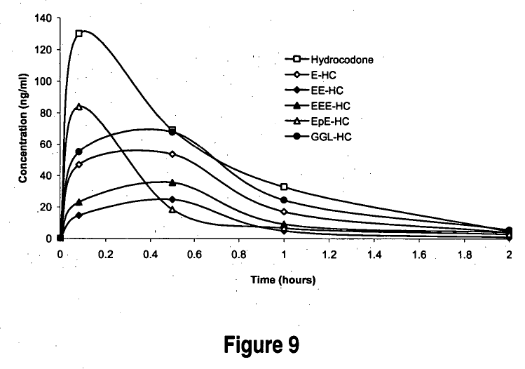

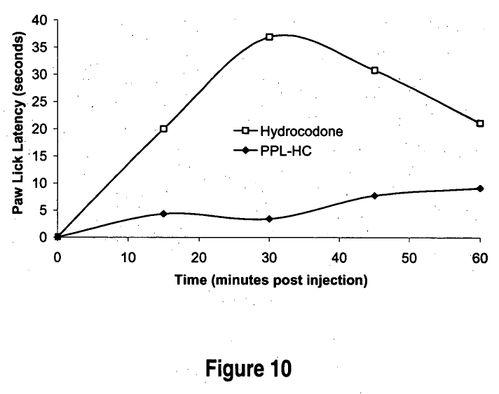

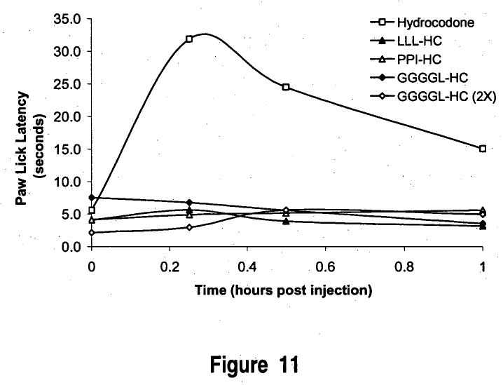

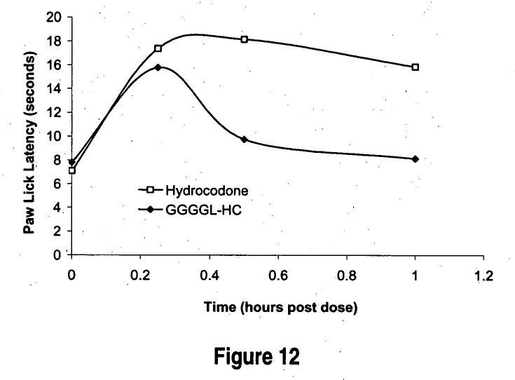

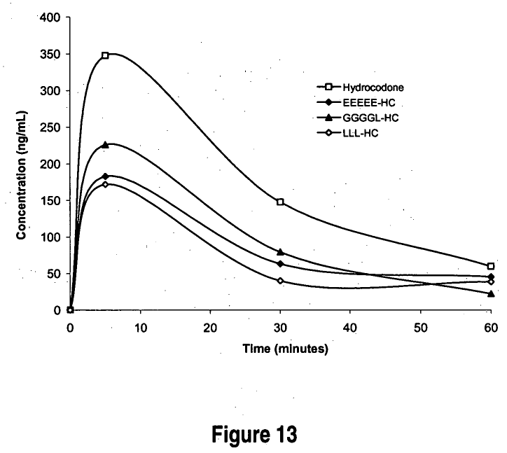

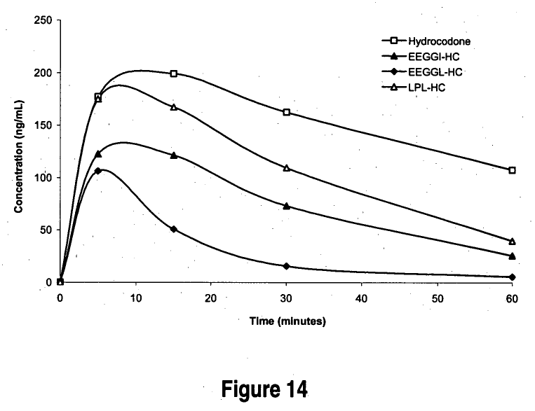

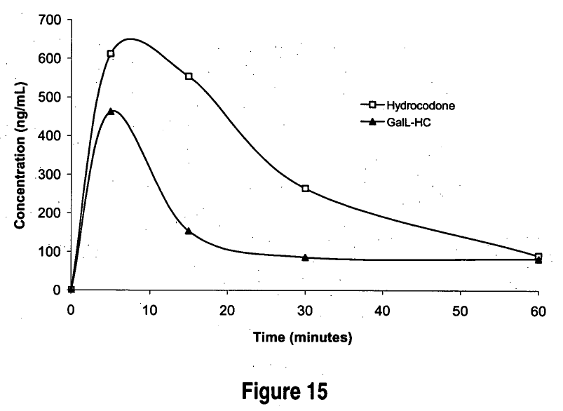

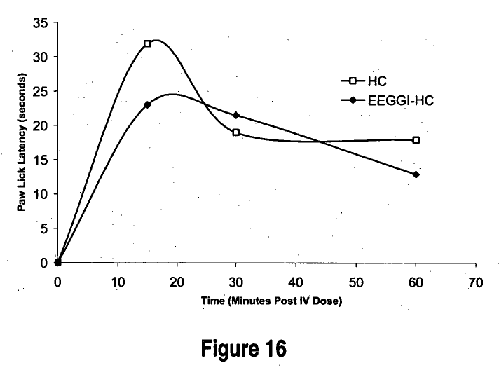

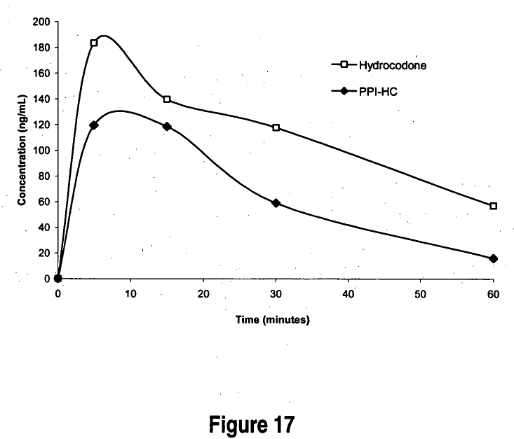

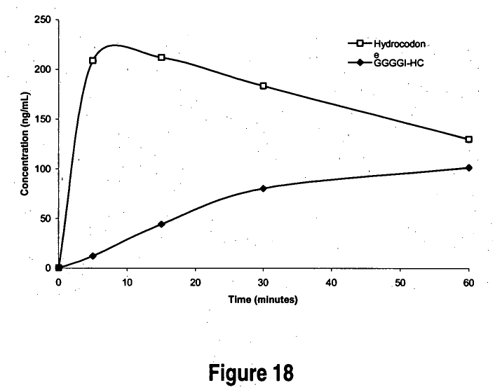

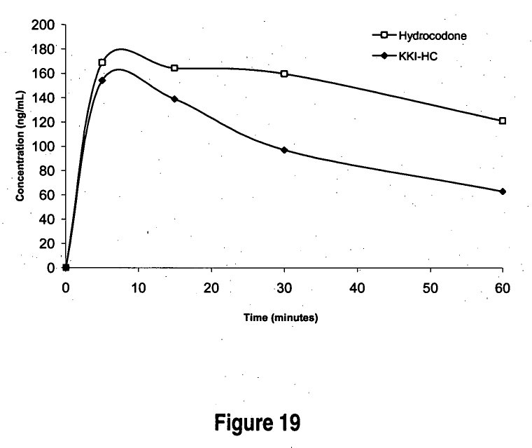

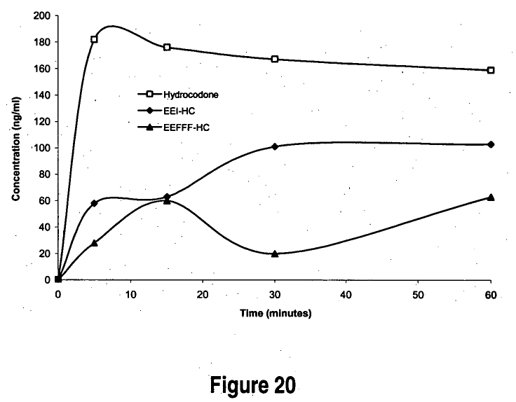

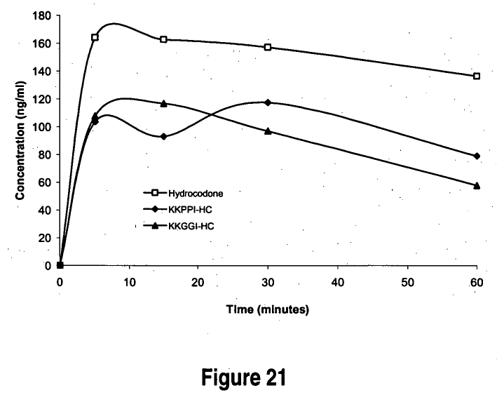

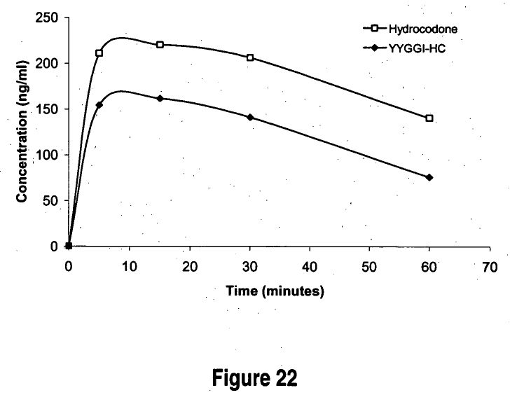

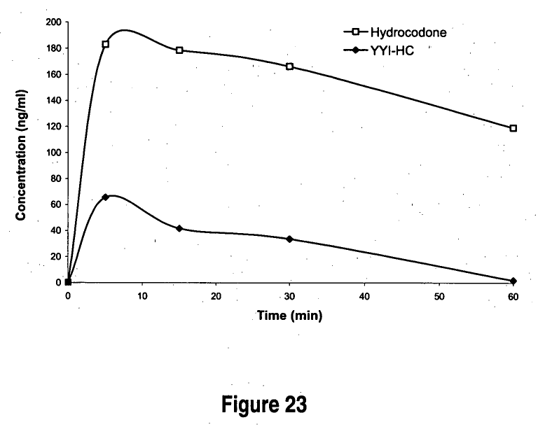

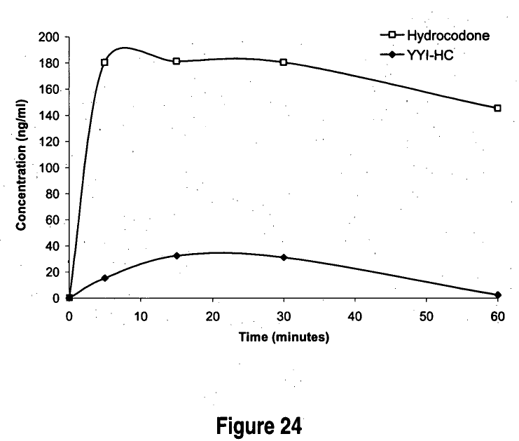

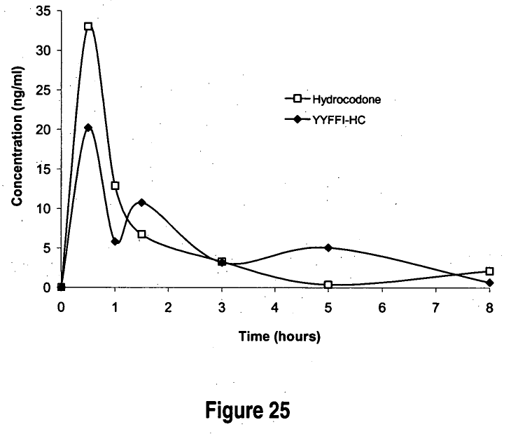

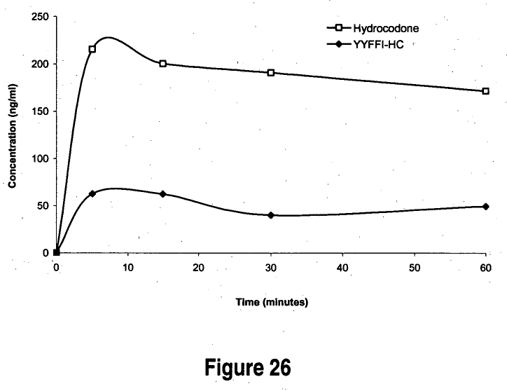

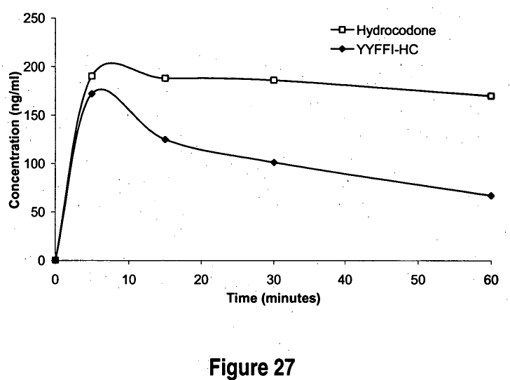

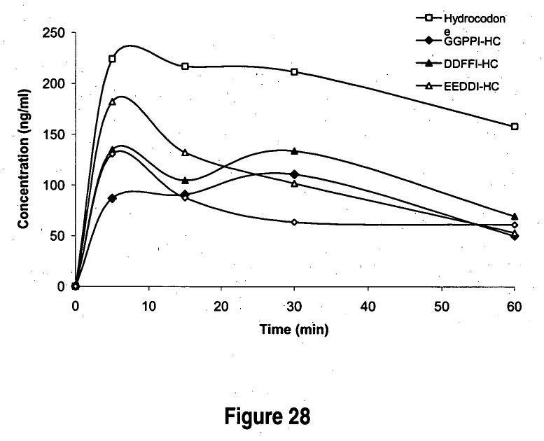

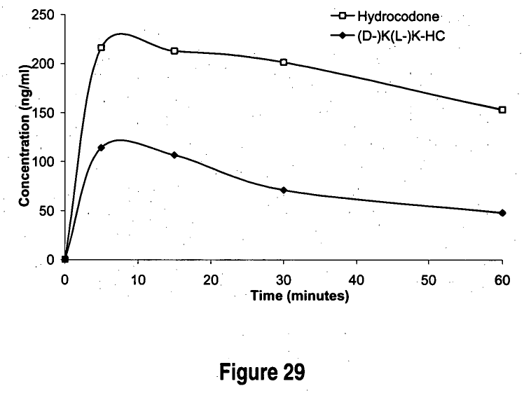

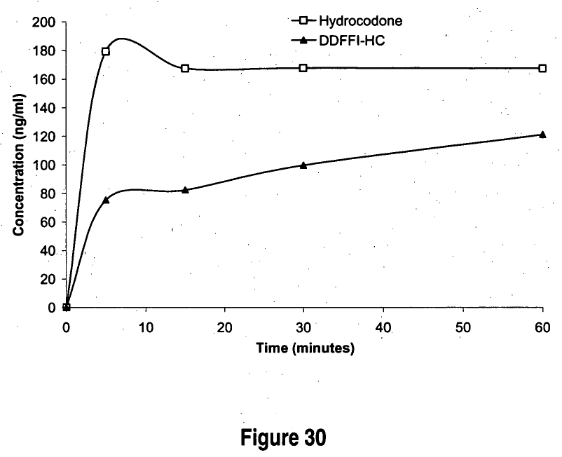

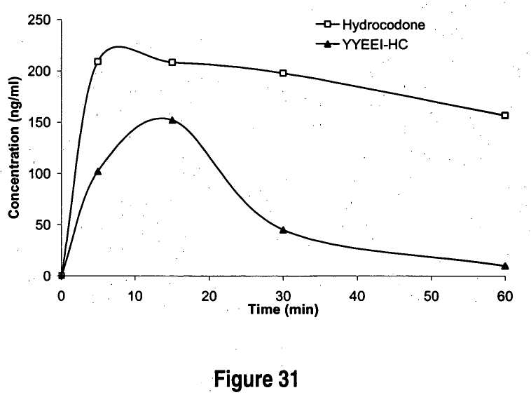

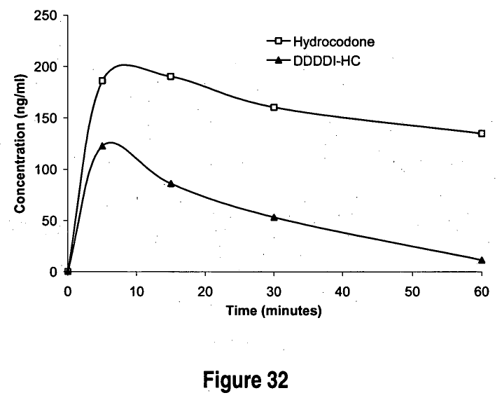

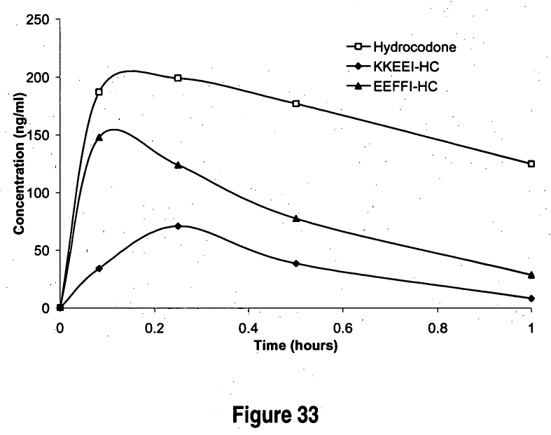

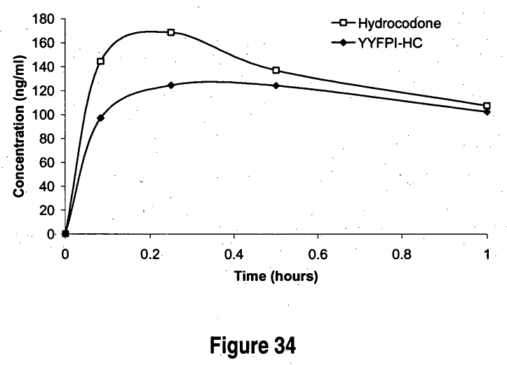

FIGS. 9 through 34 demonstrate plasma levels measured by ELISA of various compounds described in Examples 3 through 36.

Glycopeptides

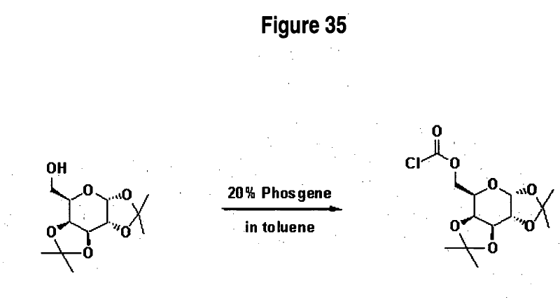

FIG. 35 illustrates preparation of 1,2:3,4-di-O-isopropylidene-D-galactopyranose.

| | | | | | Molar |
|---|---|---|---|---|---|
| Reagents | MW | Weight | | mmoles | Equivalents |
| 1,2:3,4-di-O-isopropylidene- | 260 | 1.00 | g | 3.85 | 1 |
| D-galactopyranose | | | | | |
| 20% Phosgene in toluene | — | 20 | ml | — | — |

Chloroformate of 1,2:3,4-di-O-isopropylidene-D-galactopyranose

To a stirring solution of 20% phosgene in toluene under an inert atmosphere was added 1,2:3,4-di-O-isopropylidene-D-galactopyranose via syringe. The resulting clear, colorless solution was stirred at ambient temperature for 30 minutes. After stirring, Ar(g) was bubbled through the solution for approximately 20 minutes to remove any excess phosgene. Solvent was then removed and product dried under vacuum for 18 hours. Product was used without further purification or characterization.

### Example 37

### Galactose-CO-Leu-Hydrocodone

Synthesis of Galactose-CO-Leu-Hydrocodone

To the chloroformate of galactose (1.5 eq) in dimethylformamide (DMF) (2 ml/mmol) was added Leu-Hydrocodone (1 eq) and 4-methylmorpholine (NMM) (6 eq). The reaction was stirred at ambient temperatures for 18 hours. Reaction was quenched by the addition of water, solvents were removed and crude product was isolated by purification with reverse-phase HPLC.

Product was deprotected using 1:1 1M HCl: THF (1 ml/0.1 mmol) in 3 hours. Product was re-purified by reverse-phase HPLC.

### Example 38

### Galactose-CO-Pro2-Ile-Hydrocodone

Synthesis of Galactose-CO-Pro₂-Ile-Hydrocodone

Galactose-CO-Pro₂-Ile-Hydrocodone was prepared in a manner similar to Example 37 except Pro₂-Ile-Hydrocodone was used as the conjugated starting material.

### Example 39

### Galactose-CO-Pro2-Leu-Hydrocodone

Synthesis of Galactose-CO-Pro₂-Leu-Hydrocodone

Galactose-CO-Pro₂-Leu-Hydrocodone was prepared in a manner similar to Example 37 Pro₂-Leu-Hydrocodone was used as the conjugated starting material.

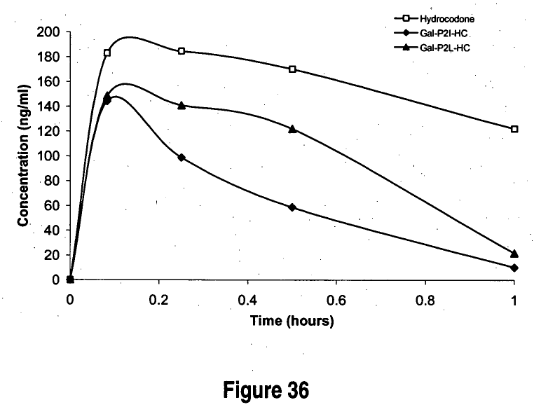

FIG. 36 illustrates oral bioavailability of abuse-resistant hydrocodone glyco-peptide conjugates, measured as free hydrocodone.

### Example 40

### Gulonic Acid-Ile-Hydrocodone

Synthesis of Gulonic Acid-Ile-Hydrocodone

Gulonic acid-Ile-Hydrocodone was prepared in a manner similar to Example 37 except Ile-Hydrocodone was used as the conjugated starting material and Gulonic acid-OSu was used as the carbohydrate starting material.

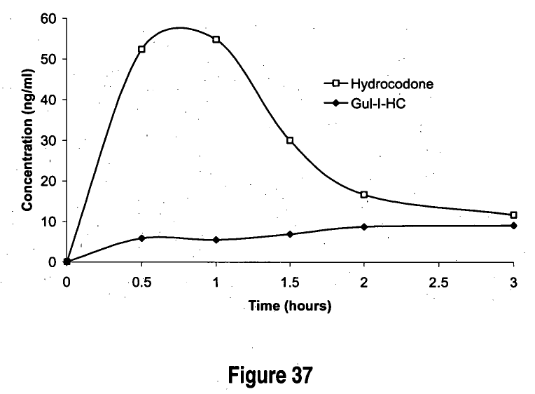

FIG. 37 illustrates Oral bioavailability of an abuse-resistant hydrocodone amino acid-carbohydrate conjugate, measured as free hydrocodone.

D-Amino Acids

### Example 41

### (d)-Lys-(1)-Lys-Ile-Hydrocodone

Preparation of (d)-Lys-(1)-Lys-Ile-Hydrocodone

To a solution of Ile-Hydrocodone in DMF was added NMM followed by Boc-(d)-Lys(Boc)-(1)-Lys(Boc)-OSu. The solution was stirred at ambient temperatures for 18 hours. Solvent was removed. Crude material was purified using preparative HPLC (Phenomenex Luna C18, 30×250 mm, 5 μM, 100 Å; Gradient: 90 water/10 0.1% TFA-MeCN→0/100; 30 ml/min.). Solid was collected as a slightly yellow powder. To the Boc-(d)-Lys(Boc)-(1)-Lys(Boc)-Hydrocodone was added 4N HCl in dioxane. The resulting mixture was stirred at ambient temperatures for 18 hours. Solvent was removed and final product dried under vacuum. Solid was collected as a slightly yellow solid.

Nucleosides

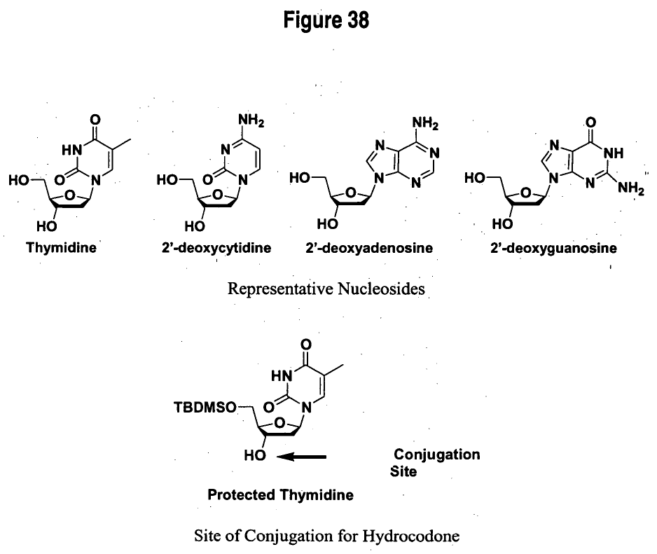

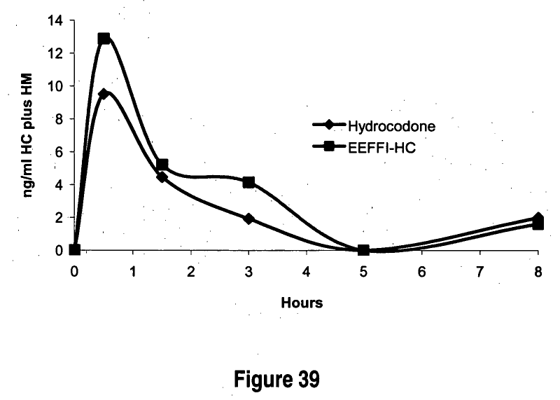

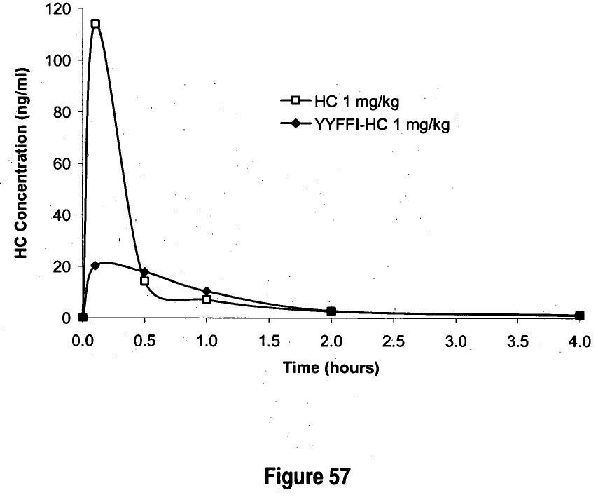

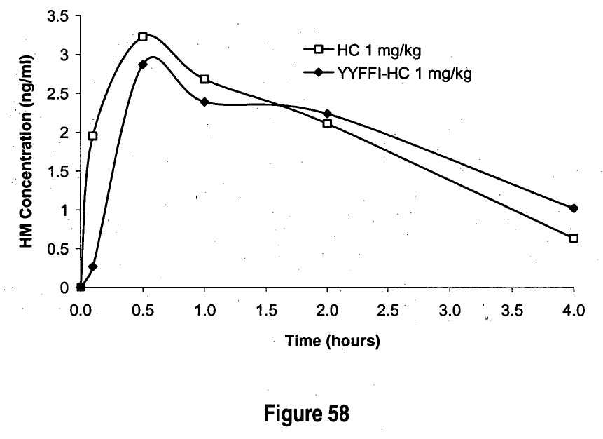

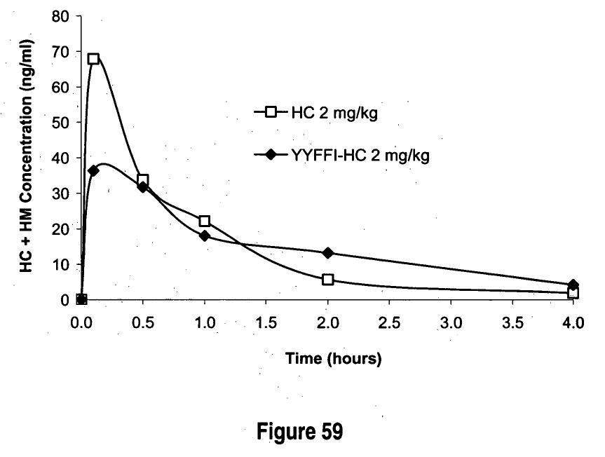

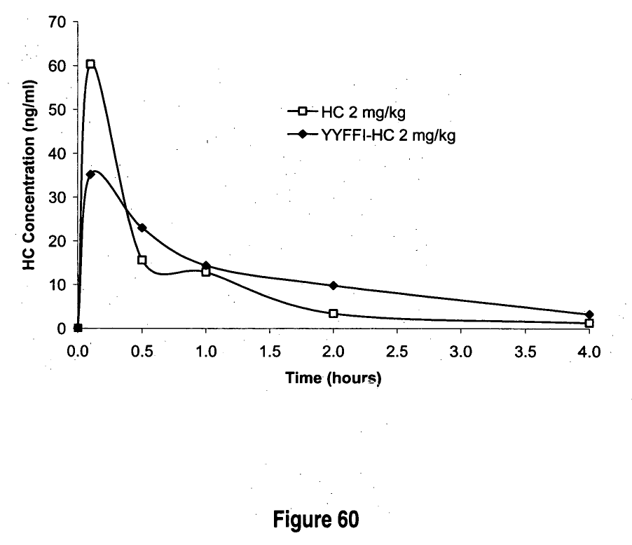

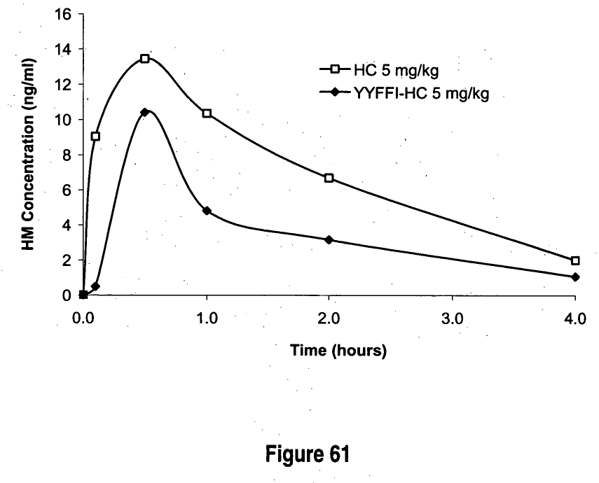

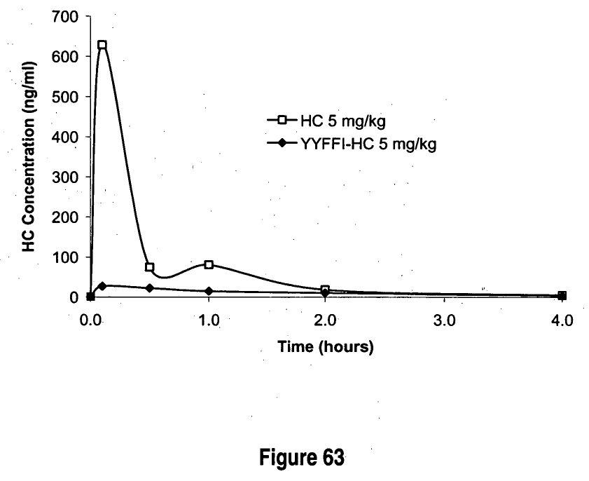

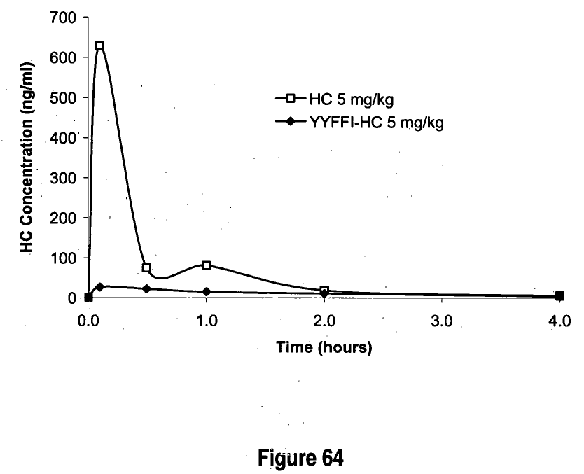

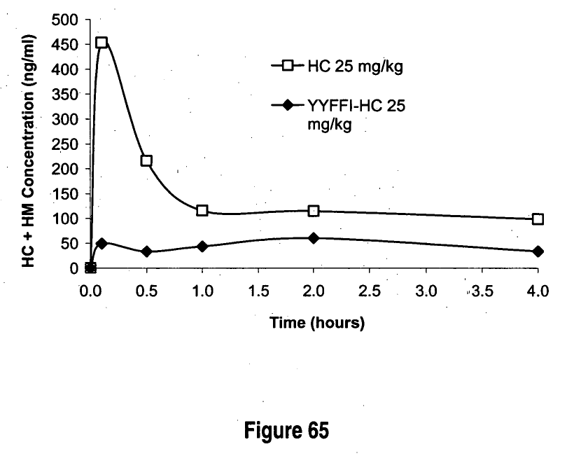

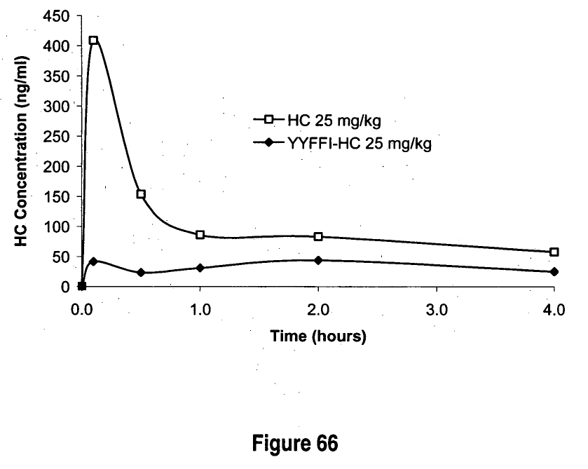

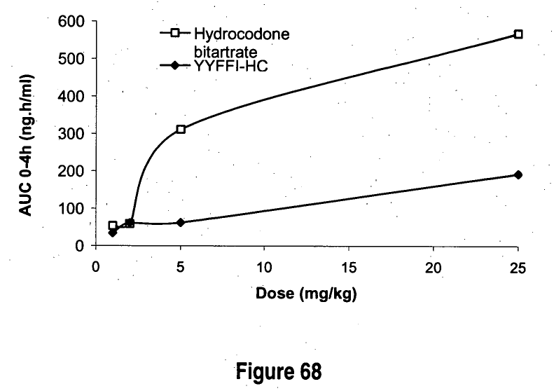

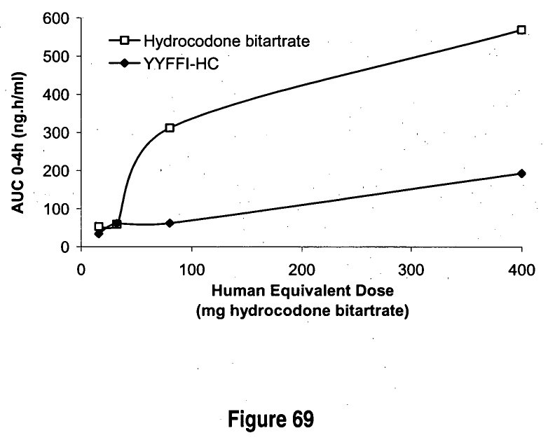

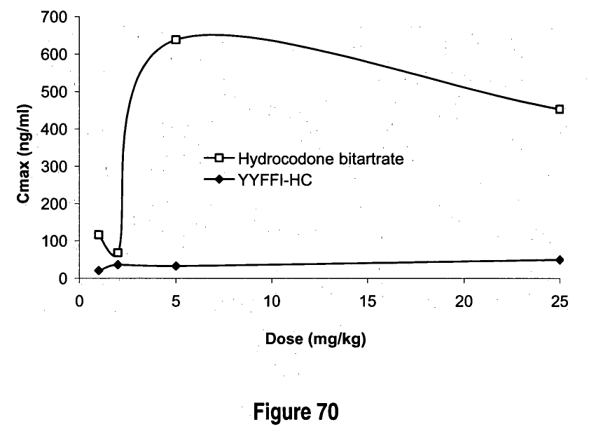

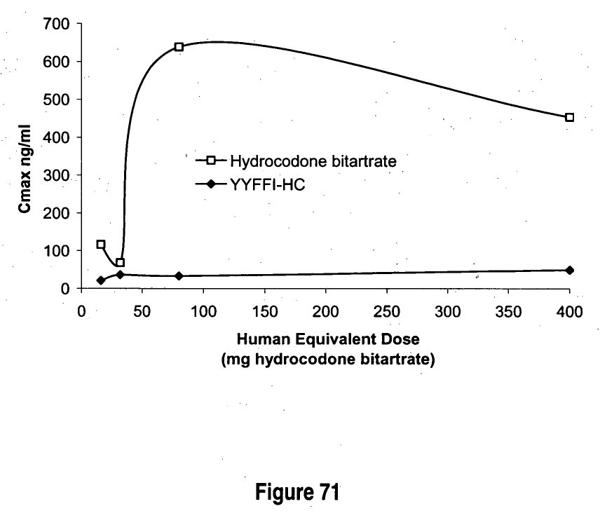

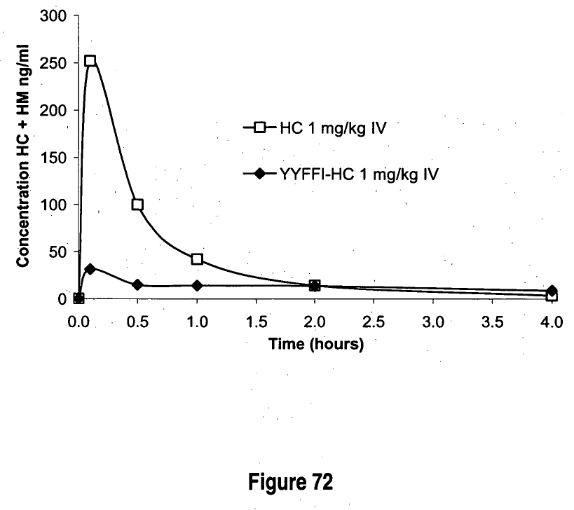

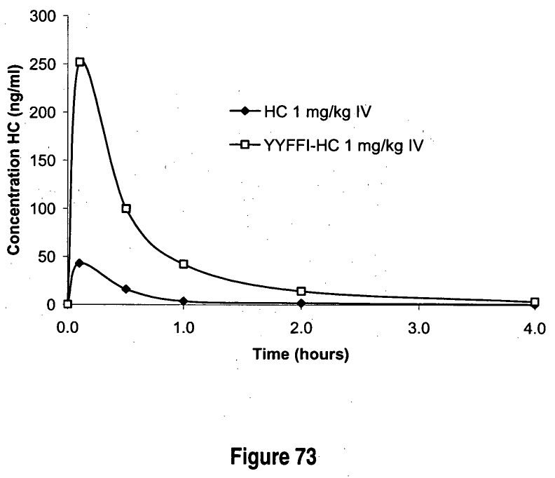

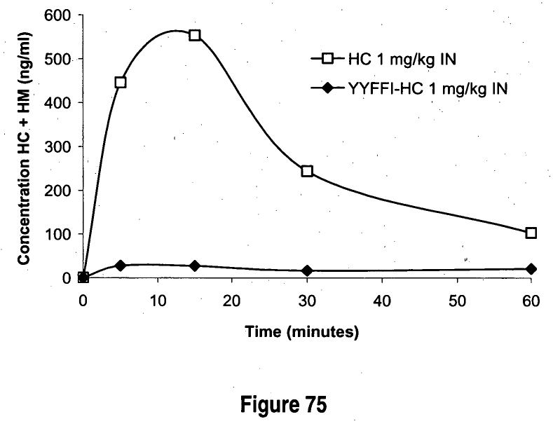

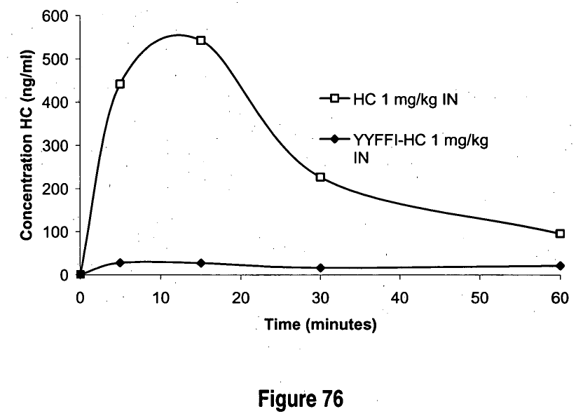

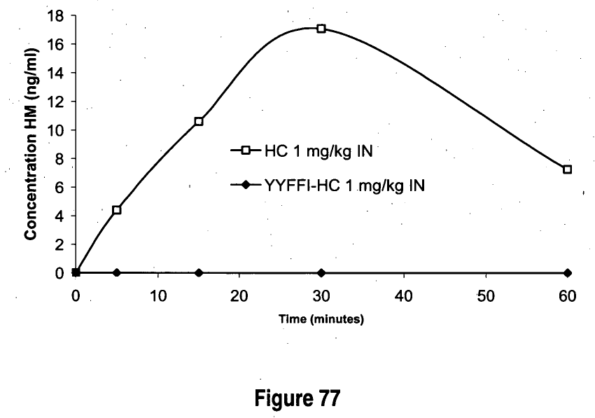

FIG. 38 illustrates nucleosides and conjugation sites. Examples 42 through 51 are also described through FIGS. 39 through 77 (with plasma levels measured by LC/MS/MS).

### Example 42

### Oral Bioavailability of Peptide-Hydrocodone Conjugates at a Dose (1 mg/kg) Approximating a Therapeutic Human Dose and at an Elevated Dose

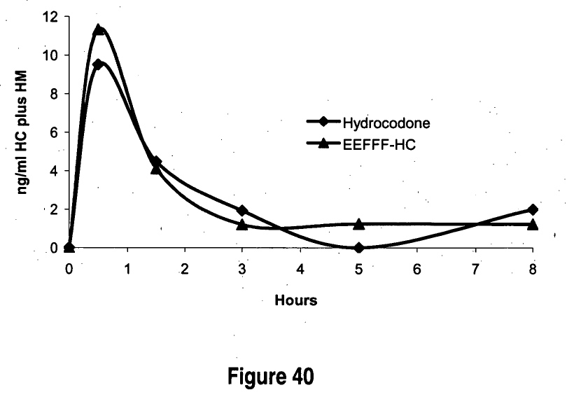

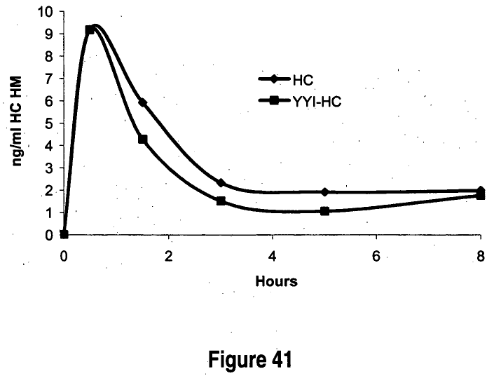

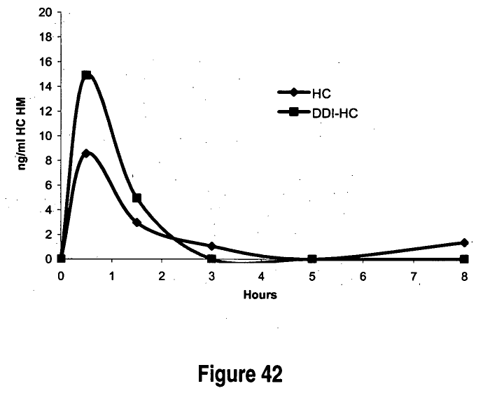

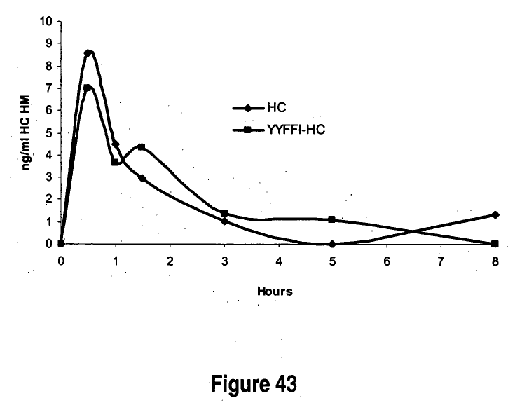

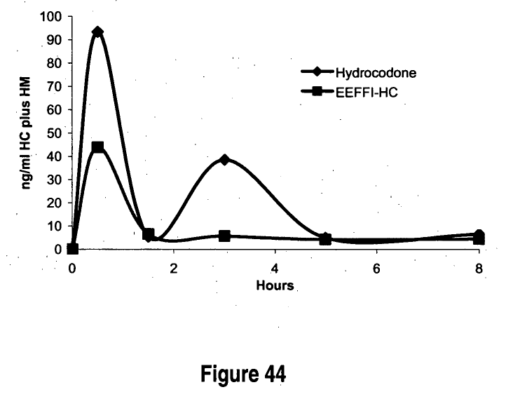

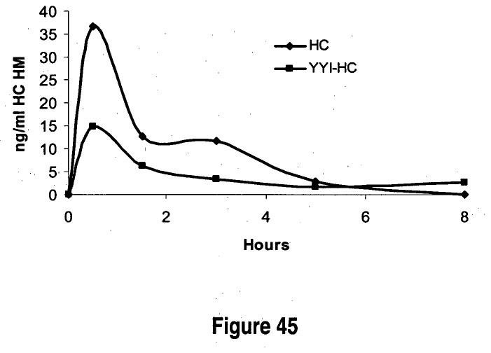

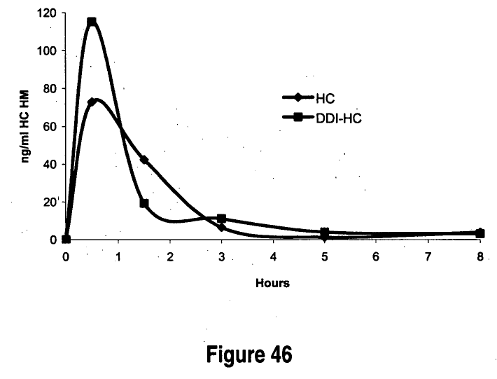

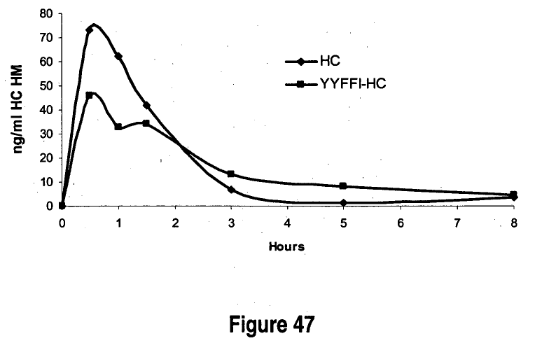

Example 42 illustrates that when the peptides EEFFI (Table 1, FIG. 39), EEFFF (Table 2, FIG. 40), YYI (Table 3, FIG. 41), DDI (Table 4, FIG. 42), and YYFFI (Table 5, FIG. 43) are conjugated to the active agent hydrocodone oral bioavailability is maintained or increased over an equivalent hydrocodone dose when the dose is administered as 1 mg/kg. This dose is the equivalent of a human dose of 10 to 14 mg for an individual weighing 70 kg (148 lbs) according to Chou et al. However, when administered orally at 5 mg/kg peak levels and bioavailability of EEFFI-HC (Table 6, FIG. 44), YYI-HC (Table 7, FIG. 45), DDI-HC (Table 8, FIG. 46) and YYFFI-HC (Table 9, FIG. 47) are substantially decreased. A 5 mg/kg dose in rats approximates an 80 mg human equivalent dose (HED) of hydrocodone bitartrate; a dose that would be likely to be harmful to a naïve patient in immediate release form with the potential for fatal overdose. Human equivalent doses are defined as the equivalent dose for a 60 kg person adjusted for the body surface area of the animal model. The adjustment factor for rats is 6.2. The HED for a rat dose of 5 mg/kg of hydrocodone base, for example, is equivalent to 48.39 mg (5/6.2×60) hydrocodone base; which is equivalent to 79.98 (48.39/0.605) mg hydrocodone bitartrate, when adjusted for the salt content.

Thus the peptide-hydrocodone conjugates maintain their therapeutic value at the lower dose (1 mg/kg), whereas when given at a dose above a safe level (5 mg/kg) bioavailability is decreased as compared to hydrocodone, thus diminishing the potential for overdose by oral ingestion. The decrease in bioavailability of hydrocodone from peptide hydrocodone conjugates relative to hydrocodone ranged from 9 to 70 percent (Table 10).

TABLE 1
Oral Pharmacokinetics of Hydrocodone vs. EEFFI-HC (1 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | HC | ng/ml | HC |
| Hydrocodone Bitartrate | 9.5 | 4.5 | 1.9 | 0 | 2 | 19.1 | 100 | 9.5 | 100 |
| EEFFI-HC | 12.9 | 5.2 | 4.2 | 0 | 1.6 | 25.8 | 135 | 12.9 | 136 |

hydrocodone plus hydromorphone (ng/ml)

TABLE 2
Oral Pharmacokinetics of Hydrocodone vs. EEFFF-HC (1 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | HC | ng/ml | HC |
| Hydrocodone Bitartrate | 9.5 | 4.5 | 1.9 | 0 | 2 | 19.1 | 100 | 9.5 | 100 |
| EEFFF-HC | 11.3 | 4.1 | 1.2 | 1.2 | 1.2 | 20.7 | 108 | 11.3 | 119 |

hydrocodone plus hydromorphone (ng/ml)

TABLE 3
Oral Pharmacokinetics of Hydrocodone vs. YYI-HC (1 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | HC | ng/ml | HC |
| Hydrocodone Bitartrate | 9.2 | 5.9 | 2.3 | 1.9 | 2 | 26.1 | 100 | 9.2 | 100 |
| YYI-HC | 9.2 | 4.3 | 1.5 | 1.1 | 1.8 | 20.4 | 78 | 9.2 | 100 |

hydrocodone plus hydromorphone (ng/ml)

TABLE 4
Oral Pharmacokinetics of Hydrocodone vs. DDI-HC (1 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | HC | ng/ml | HC |
| Hydrocodone Bitartrate | 8.6 | 3 | 1.1 | 0 | 1.4 | 14 | 100 | 8.6 | 100 |
| DDI-HC | 14.9 | 5 | 0 | 0 | 0 | 17.4 | 124 | 14.9 | 173 |

hydrocodone plus hydromorphone (ng/ml)

TABLE 5
Oral Pharmacokinetics of Hydrocodone vs. YYFFI-HC (1 mg/kg dose).

| | Hours | | | | | | AUC (ng/ml h) | Percent | Cmax | Percent |
|---|---|---|---|---|---|---|---|---|---|---|
| Drug | 0.5 | 1.0 | 1.5 | 3 | 5 | 8 | 0-8 h | HC | ng/ml | HC |
| Hydrocodone Bitartrate | 8.6 | 4.5 | 3 | 1.1 | 0 | 1.4 | 13.6 | 100 | 8.6 | 100 |
| YYFFI-HC | 7 | 3.7 | 4.3 | 1.4 | 1.1 | 0 | 14.9 | 110 | 7 | 81 |

hydrocodone plus hydromorphone (ng/ml)

TABLE 6
Oral Pharmacokinetics of Hydrocodone vs. EEFFI-HC (5 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | HC | ng/ml | HC |
| Hydrocodone Bitartrate | 93 | 5.3 | 39 | 5 | 6.5 | 167 | 100 | 93 | 100 |
| EEFFI-HC | 44 | 6.5 | 5.7 | 4.2 | 4.5 | 68 | 41 | 44 | 47 |

hydrocodone plus hydromorphone (ng/ml)

TABLE 7
Oral Pharmacokinetics of Hydrocodone vs. YYI-HC (5 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | HC | ng/ml | HC |
| Hydrocodone Bitartrate | 37 | 13 | 12 | 3 | 0 | 71 | 100 | 37 | 100 |
| YYI-HC | 15 | 6.3 | 3.3 | 1.6 | 2.7 | 33 | 46 | 15 | 41 |

hydrocodone plus hydromorphone (ng/ml)

TABLE 8
Oral Pharmacokinetics of Hydrocodone vs. DDI-HC (5 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | HC | ng/ml | HC |
| Hydrocodone Bitartrate | 73 | 42 | 6.7 | 1.2 | 3.8 | 128 | 100 | 73 | 100 |
| DDI-HC | 115 | 19 | 11 | 4 | 3.1 | 145 | 113 | 115 | 158 |

hydrocodone plus hydromorphone (ng/ml)

TABLE 9
Oral Pharmacokinetics of Hydrocodone vs. YYFFI-HC (5 mg/kg dose).

| | Hours | | | | | | AUC (ng/ml h) | Percent | Cmax | Percent |
|---|---|---|---|---|---|---|---|---|---|---|
| Drug | 0.5 | 1.0 | 1.5 | 3 | 5 | 8 | 0-8 h | HC | ng/ml | HC |
| Hydrocodone Bitartrate | 73 | 62 | 42 | 6.7 | 1.2 | 3.8 | 123 | 100 | 73 | 100 |
| YYFFI-HC | 46 | 33 | 34 | 13 | 8.3 | 4.5 | 105 | 86 | 46 | 63 |

hydrocodone plus hydromorphone (ng/ml)

TABLE 10
Decrease in Oral Bioavailability at 5 mg/kg vs.
Therapeutic Dose of 1 mg/kg.

| | | | | | Percent | |
|---|---|---|---|---|---|---|
| | Bio- | | Bio- | | Decrease | |
| | availability | | availability | | 1 mg/kg vs. | |
| | 1 mg/kg | | 5 mg/kg | | 5 mg/kg | |
| Drug | AUC | Cmax | AUC | Cmax | AUC | Cmax |
| YYI-HC | 78 | 100 | 46 | 40 | 41 | 60 |
| DDI-HC | 124 | 174 | 113 | 158 | 9 | 9 |
| YYFFI-HC | 109 | 81 | 86 | 62 | 15 | 23 |
| EEFFI-HC | 135 | 136 | 41 | 47 | 70 | 65 |

### Example 43

### Bioavailability of Peptide-HC Conjugates by the Intranasal Route

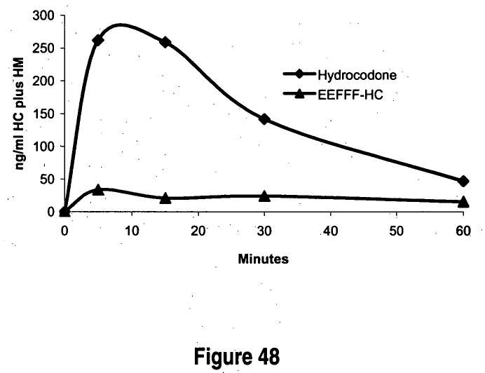

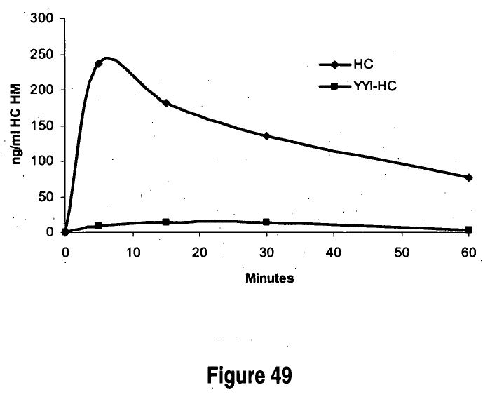

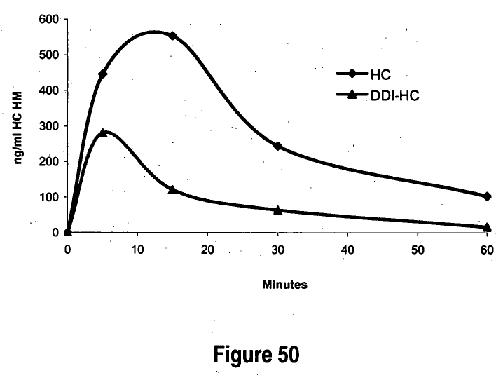

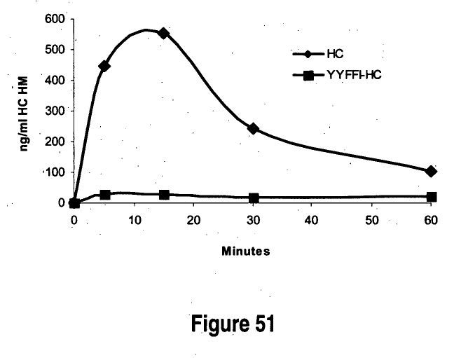

Example 43 illustrates that when the peptides EEFFF (Table 11, FIG. 48), YYI (Table 12, FIG. 49), DDI (Table 13, FIG. 50) and YYFFI (Table 14, FIG. 51) are conjugated to the active agent hydrocodone the bioavailability by the intravenous route is substantially decreased thereby diminishing the possibility of overdose when the drug is administered by snorting.

TABLE 11
Intranasal Pharmacokinetics of Hydrocodone vs. EEFFF-HC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 262 | 259 | 142 | 47 | 152 | 100 | 262 | 100 |
| EEFFF-HC | 34 | 21 | 24 | 15 | 21 | 14 | 34 | 13 |

hydrocodone plus hydromorphone (ng/ml)

TABLE 12
Intranasal Pharmacokinetics of Hydrocodone vs. YYI-HC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 446 | 553 | 244 | 103 | 288 | 100 | 553 | 100 |
| YYI-HC | 31 | 17 | 12 | 2 | 12 | 4 | 31 | 6 |

hydrocodone plus hydromorphone (ng/ml)

TABLE 13
Intranasal Pharmacokinetics of Hydrocodone vs. DDI-HC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 446 | 553 | 244 | 103 | 288 | 100 | 553 | 100 |
| DDI-HC | 281 | 121 | 64 | 16 | 88 | 31 | 281 | 51 |

hydrocodone plus hydromorphone (ng/ml)

TABLE 14
Intranasal Pharmacokinetics of Hydrocodone vs. YYFFI-HC (1 mg/kg dose).

| | HC,Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 446 | 553 | 244 | 103 | 288 | 100 | 553 | 100 |
| YYFFI-HC | 28 | 27 | 16 | 21 | 20 | 100 | 28 | 5 |

hydrocodone plus hydromorphone (ng/ml)

### Example 44

### Bioavailability of Peptide-HC Conjugates by the Intravenous Route

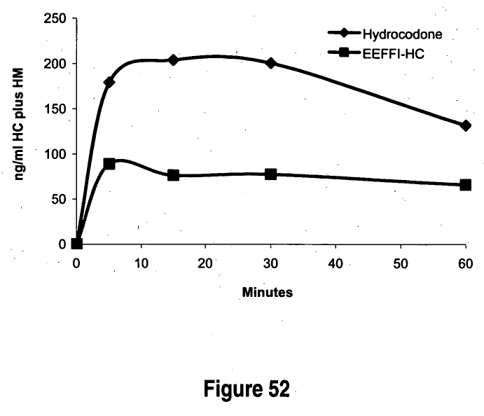

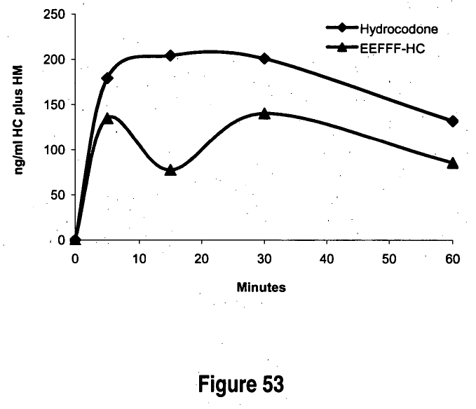

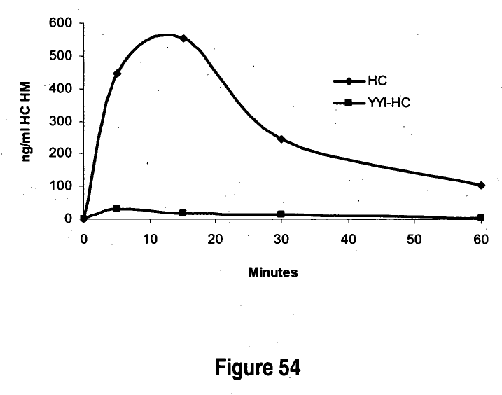

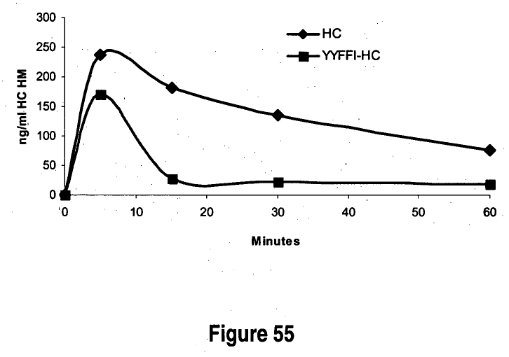

Example 44 illustrates that when the peptides EEFFI (Table 15, FIG. 52), EEFFF (Table 16, FIG. 53), YYI (Table 17, FIG. 54) and YYFFI (Table 18, FIG. 55) are conjugated to the active agent hydrocodone the bioavailability by the intravenous route is substantially decreased thereby diminishing the possibility of overdose when the drug is administered by this unintended route.

TABLE 15
Intravenous Pharmacokinetics of Hydrocodone vs. EEFFI-HC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 179 | 204 | 201 | 132 | 173 | 100 | 179 | 100 |
| EEFFI-HC | 89 | 76 | 78 | 66 | 66 | 38 | 89 | 44 |

hydrocodone plus hydromorphone (ng/ml)

TABLE 16
Intravenous Pharmacokinetics of Hydrocodone vs. EEFFF-HC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 179 | 204 | 201 | 132 | 173 | 100 | 179 | 100 |
| EEFFF-HC | 135 | 77 | 140 | 85 | 107 | 62 | 135 | 75 |

hydrocodone plus hydromorphone (ng/ml)

TABLE 17
Intravenous Pharmacokinetics of Hydrocodone vs. YYI-HC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 238 | 182 | 136 | 77 | 138 | 100 | 238 | 100 |
| YYI-HC | 9 | 13 | 13 | 3 | 10 | 7 | 13 | 6 |

hydrocodone plus hydromorphone (ng/ml)

TABLE 18
Intravenous Pharmacokinetics of Hydrocodone vs. YYFFI-HC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | HC | ng/ml | HC |
| Hydrocodone Bitartrate | 238 | 182 | 136 | 77 | 138 | 100 | 238 | 100 |
| YYFFI-HC | 171 | 28 | 22 | 18 | 40 | 29 | 171 | 72 |

hydrocodone plus hydromorphone (ng/ml)

### Example 45

### Hydrocodone Conjugates

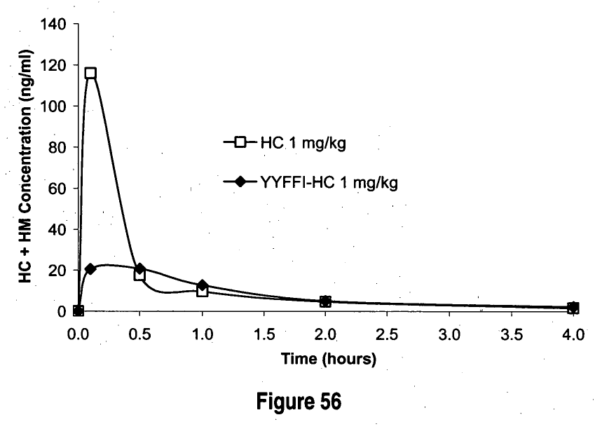

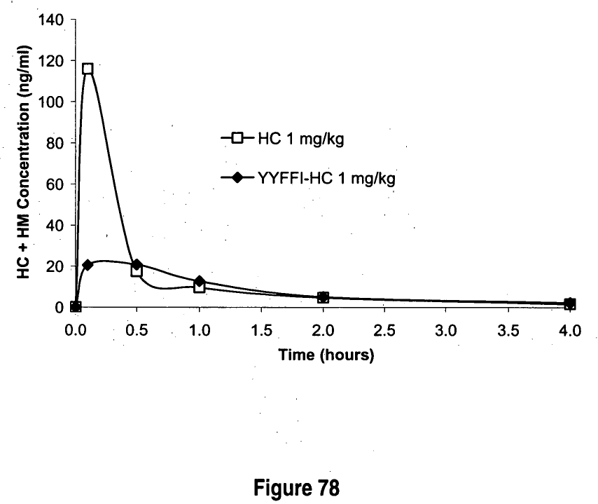

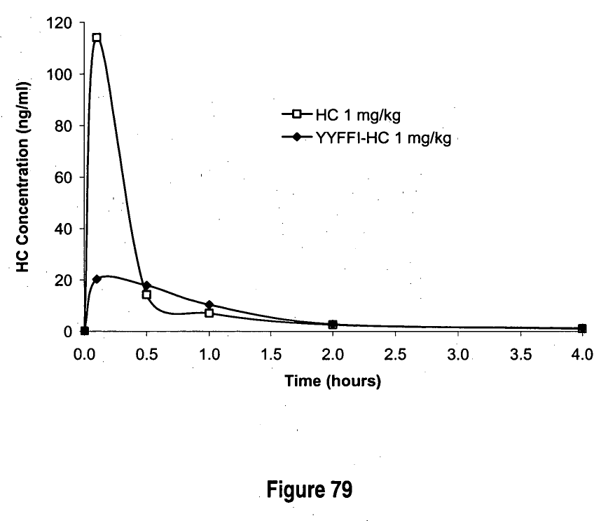

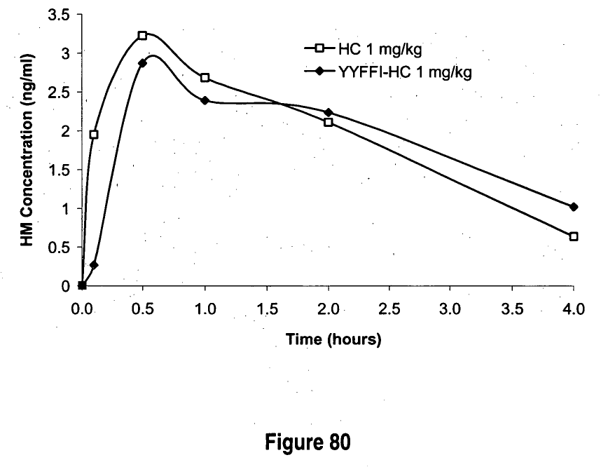

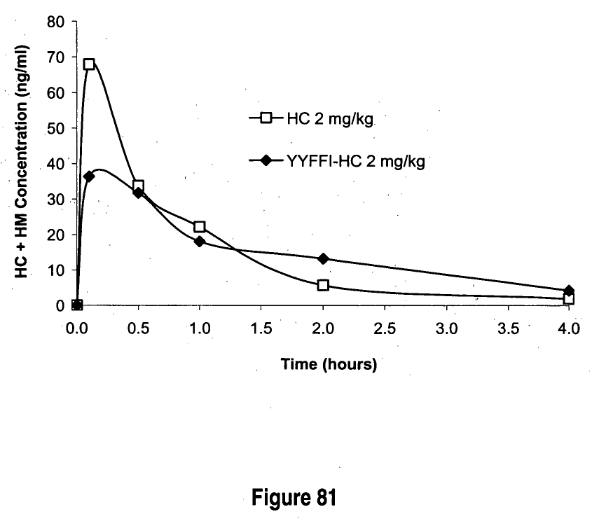

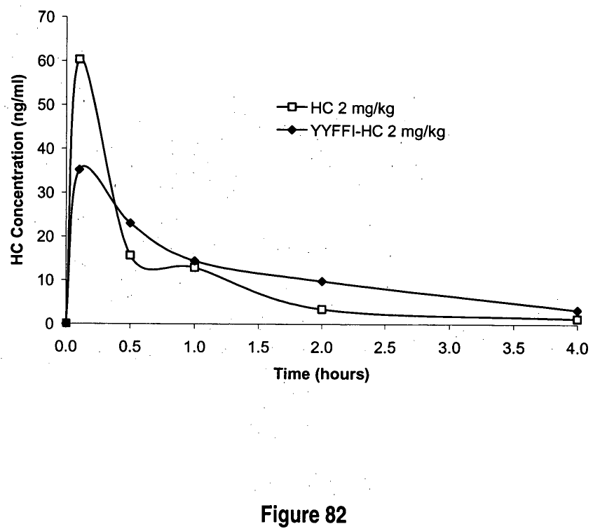

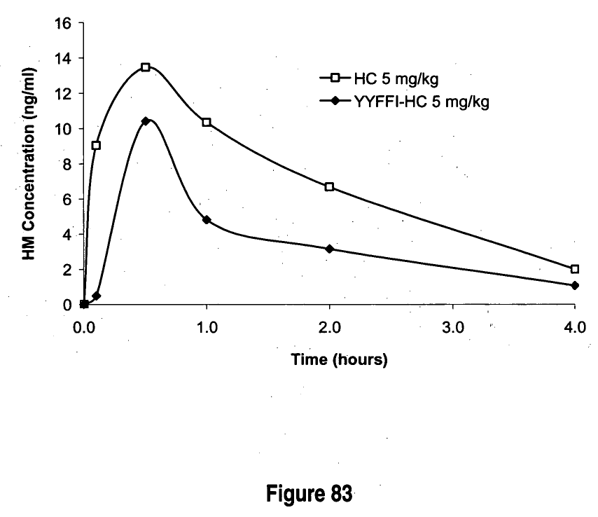

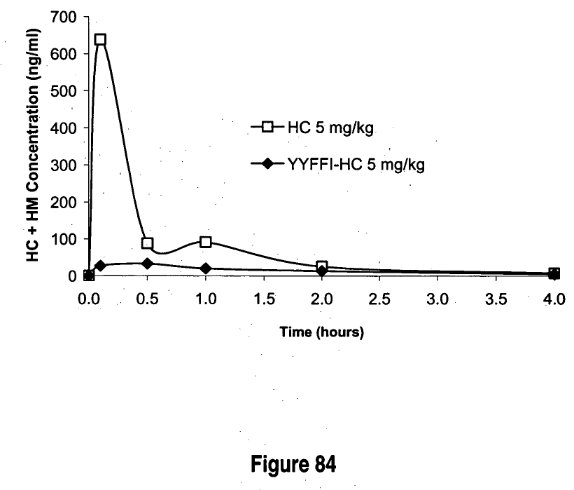

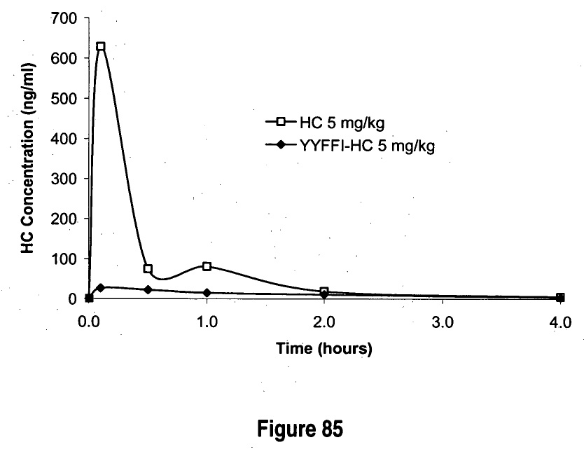

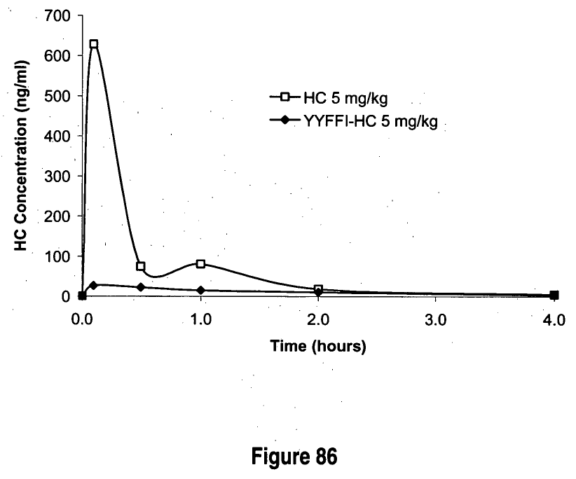

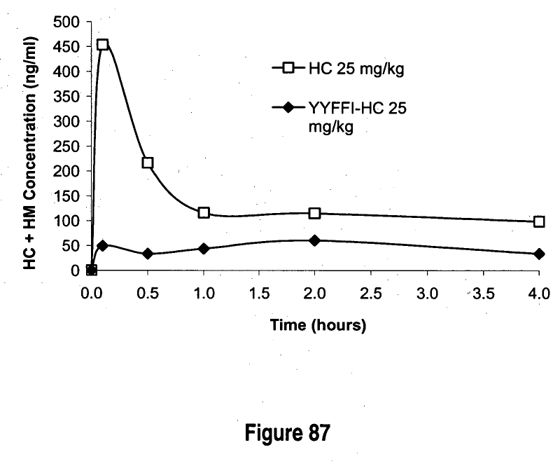

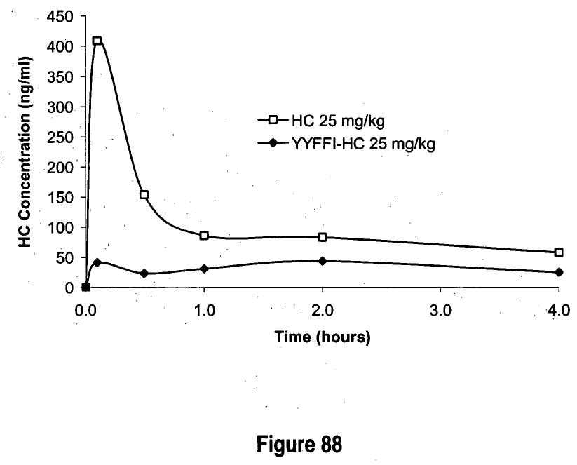

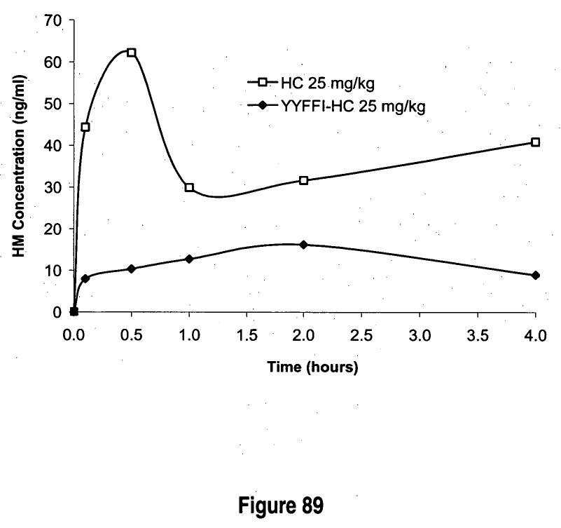

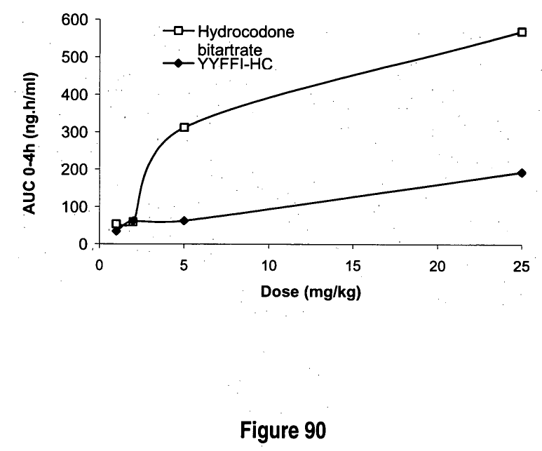

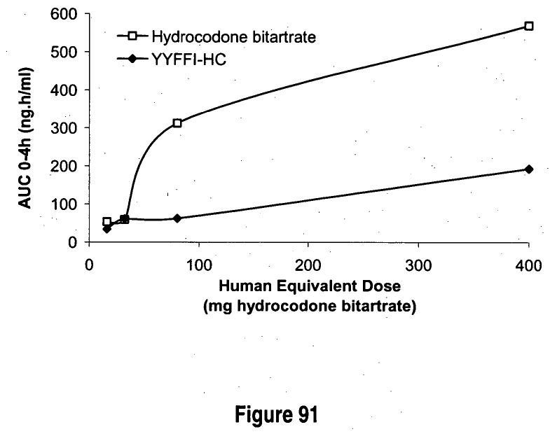

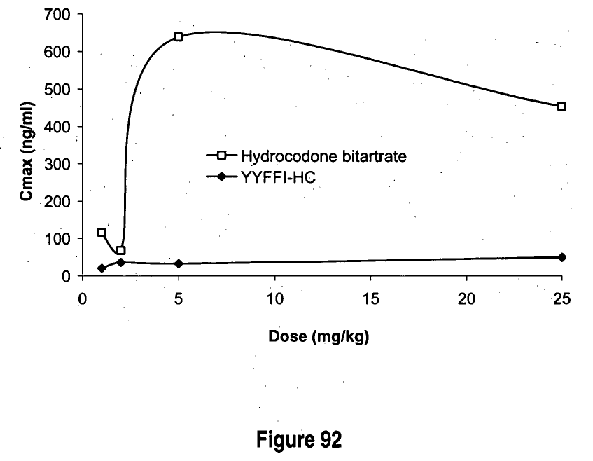

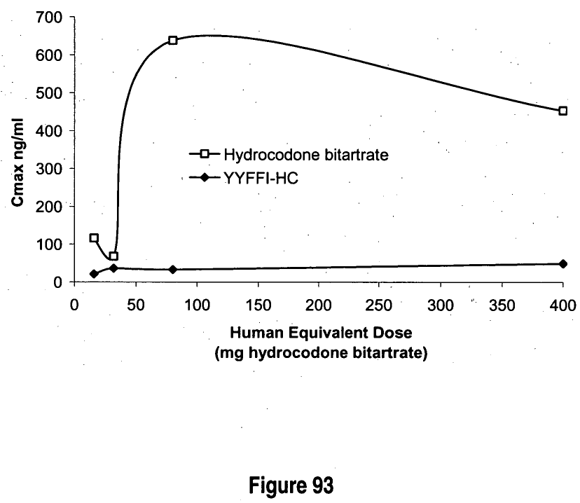

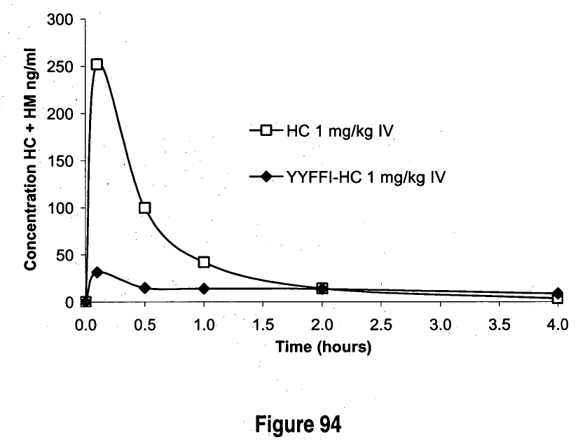

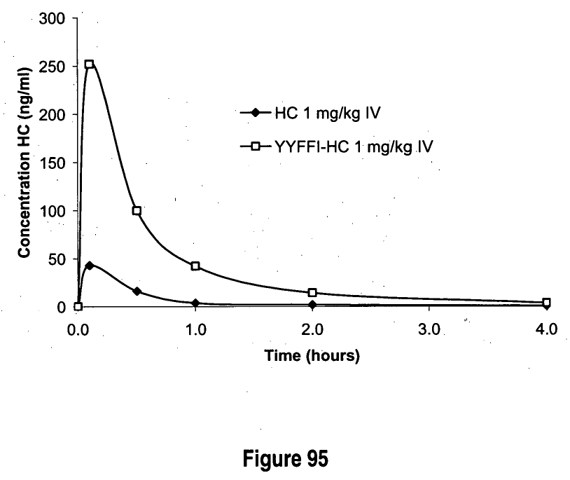

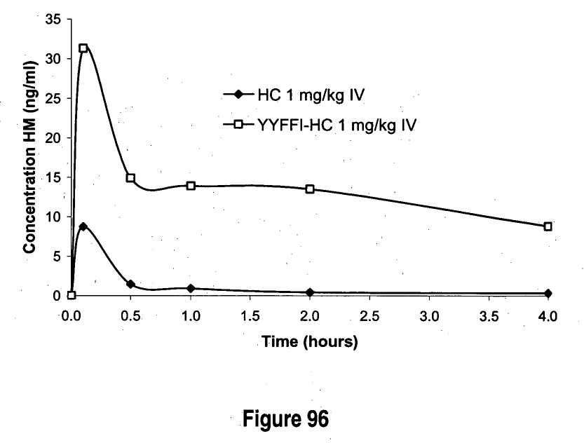

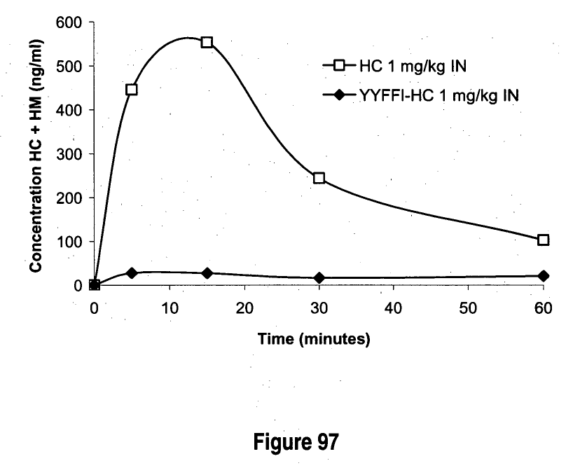

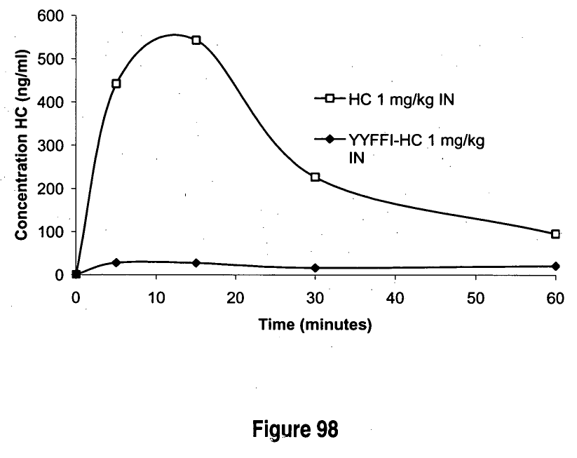

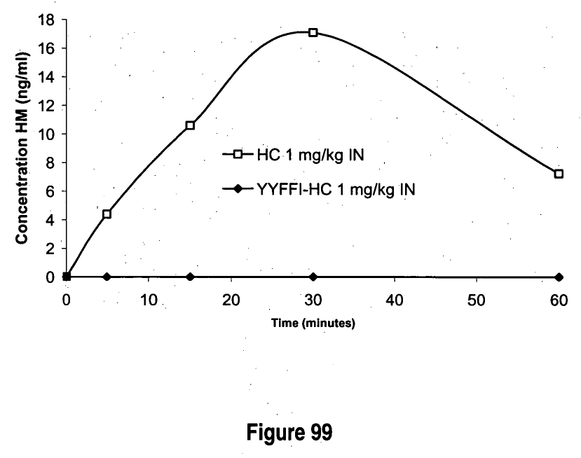

Bioavailability (AUC and Cmax) of various peptide-hydrocodone conjugates relative to that of hydrocodone bitartrate are shown in Table 19. The invention is well illustrated by the in vivo performance of YYFFI-HC (FIGS. 56 through 77). At the relatively low doses of 1 and 2 mg/kg (human equivalent doses (HEDs) of 16 and 32 mg hydrocodone bitartrate) YYFFI-HC showed comparable bioavailability to that of hydrocodone bitartrate (Table 20, FIGS. 78 through 83). At the elevated doses of 5 and 25 mg/kg bioavailability of hydrocodone and hydromorphone were substantially decreased as compared to that of hydrocodone (Table 21, FIGS. 84 through 99). These doses (HED of 80 and 400 mg hydrocodne bitartrate) are equivalent to amounts well above the available prescription doses of hydrocodone bitartrate which range from 2.5 to 10 mg. When delivered by the parentaral routes of intravenous and intranasal administration a substantial decrease in bioavailability of hydrocodone and hydromorphone from YYFFI-HC as compared to hydrocodone bitratrate was observed. These examples establish that covalent modification of an opiod via attachment of a peptide provides a method of delivering bioequivalent doses when given at doses approximating a normal prescribed dose. When administered by parenteral routes or at oral doses in excess of the intended prescription the bioavailability is substantially decreased. Collectively, the examples clearly illustrate the utility of the invention for decreasing the abuse potential of opiods.

TABLE 19
Mean hydrocodone concentrations following oral administration of
hydrocodone bitartrate or YYFFI-HC at escalating doses.

| | Dose¹/Concentration (ng/ml) | | | | | | | |
|---|---|---|---|---|---|---|---|---|
| | 1 mg/kg | | 2 mg/kg | | 5 mg/kg | | 25 mg/kg | |
| Hours | HC² | YYFFI-HC³ | HC² | YYFFI-HC³ | HC² | YYFFI-HC³ | HC² | YYFFI-HC³ |
| 0 | 0 | 0 | 0 | 0 | 0 | 0 | 0 | 0 |
| 0.1 | 114.0 | 20.3 | 60.3 | 35.2 | 628.7 | 26.6 | 408.9 | 41.4 |
| 0.5 | 14.3 | 17.9 | 15.6 | 23 | 74.3 | 22.5 | 153.9 | 23.3 |
| 1.0 | 7.0 | 10.4 | 12.9 | 14.4 | 80.8 | 15.1 | 86.2 | 31.0 |
| 2.0 | 2.6 | 2.8 | 3.4 | 9.8 | 18.4 | 10.3 | 83.3 | 43.9 |
| 4.0 | 1.0 | 1.2 | 1.3 | 3.3 | 4.9 | 3.6 | 57.8 | 25.0 |

¹hydrocodone base content
²hydrocodone bitartrate
³YYFFI-HC HCl

TABLE 20
Hydrocodone pharmacokinetic parameters following oral administration of
hydrocodone bitartrate or YYFFI-HC at escalating doses.

| | Dose¹/Concentration (ng/ml) | | | | | | | |
|---|---|---|---|---|---|---|---|---|
| | 1 mg/kg | | 2 mg/kg | | 5 mg/kg | | 25 mg/kg | |
| Parameter | HC² | YYFFI-HC³ | HC² | YYFFI-HC³ | HC² | YYFFI-HC³ | HC² | YYFFI-HC³ |
| AUC | 45.1 | 26.3 | 38.2 | 48 | 234 | 47 | 419.0 | 135.0 |
| Percent HC + HM⁴ | 100 | 58 | 100 | 126 | 100 | 20 | 100 | 32 |
| Cmax | 114.0 | 20.3 | 60.3 | 35.2 | 628.7 | 26.6 | 408.9 | 41.4 |
| Percent HC + HM⁴ | 100 | 18 | 100 | 58 | 100 | 4 | 100 | 10 |

¹hydrocodone base content
²hydrocodone bitartrate
³YYFFI-HC HCl
⁴percent relative to parameter following administration of hydrocodone bitartrate

TABLE 21
Mean hydromorphone concentrations following oral administration of
hydrocodone bitartrate or YYFFI-HC at escalating doses.

| | Dose¹/Concentration (ng/ml) | | | | | | | |
|---|---|---|---|---|---|---|---|---|
| | 1 mg/kg | | 2 mg/kg | | 5 mg/kg | | 25 mg/kg | |
| Hours | HC² | YYFFI-HC³ | HC² | YYFFI-HC³ | HC² | YYFFI-HC³ | HC² | YYFFI-HC³ |
| 0 | 0 | 0 | 0 | 0 | 0 | 0 | 0 | 0 |
| 0.1 | 1.95 | 0.27 | 7.61 | 1.13 | 9.03 | 0.49 | 44.36 | 8.00 |
| 0.5 | 3.22 | 2.87 | 18.10 | 8.74 | 13.46 | 10.41 | 62.24 | 10.35 |
| 1.0 | 2.69 | 2.39 | 9.23 | 3.63 | 10.36 | 4.82 | 29.89 | 12.70 |
| 2.0 | 2.11 | 2.24 | 2.31 | 3.41 | 6.68 | 3.17 | 31.62 | 16.22 |
| 4.0 | 0.64 | 1.02 | 0.59 | 0.88 | 2.00 | 1.07 | 40.86 | 8.98 |

¹hydrocodone base content
²hydrocodone bitartrate
³YYFFI-HC HCl

TABLE 22
Hydromorphone pharmacokinetic parameters following oral administration of
hydrocodone bitartrate or YYFFI-HC at escalating doses.

| | Dose¹/Concentration (ng/ml) | | | | | | | |
|---|---|---|---|---|---|---|---|---|
| | 1 mg/kg | | 2 mg/kg | | 5 mg/kg | | 25 mg/kg | |
| Parameter | HC² | YYFFI-HC³ | HC² | YYFFI-HC³ | HC² | YYFFI-HC³ | HC² | YYFFI-HC³ |
| AUC | 7.8 | 7.5 | 21.0 | 12.9 | 28.1 | 14.3 | 149 | 49 |
| Percent HM⁴ | 100 | 97 | 100 | 61 | 100 | 51 | 100 | 33 |
| Cmax | 3.2 | 2.9 | 18.1 | 8.7 | 13.5 | 10.4 | 44.4 | 16.2 |
| Percent HM⁴ | 100 | 89 | 100 | 48 | 100 | 77 | 100 | 37 |

¹hydrocodone base content
²hydrocodone bitartrate
³YYFFI-HC HCl
⁴percent relative to parameter following administration of hydrocodone bitartrate

TABLE 23
Mean hydrocodone plus hydromorphone concentrations following oral
administration of hydrocodone bitartrate or YYFFI-HC at escalating doses.

| | Dose¹/Concentration (ng/ml) | | | | | | | |
|---|---|---|---|---|---|---|---|---|
| | 1 mg/kg | | 2 mg/kg | | 5 mg/kg | | 25 mg/kg | |
| Hours | HC² | YYFFI-HC³ | HC² | YYFFI-HC³ | HC² | YYFFI-HC³ | HC² | YYFFI-HC³ |
| 0 | 0 | 0 | 0 | 0 | 0 | 0 | 0 | 0 |
| 0.1 | 116 | 20.6 | 67.9 | 36.3 | 637.7 | 27.1 | 453.3 | 49.4 |
| 0.5 | 17.5 | 20.;8 | 33.7 | 31.7 | 87.8 | 32.9 | 216.1 | 33.7 |
| 1.0 | 9.7 | 12.8 | 22.1 | 18.0 | 91.2 | 19.9 | 116.1 | 43.7 |
| 2.0 | 4.7 | 5.0 | 5.7 | 13.2 | 25.1 | 13.5 | 114.9 | 60.1 |
| 4.0 | 1.6 | 2.2 | 1.9 | 4.2 | 6.9 | 4.7 | 98.7 | 34.0 |

¹hydrocodone base content
²hydrocodone bitartrate
³YYFFI-HC HCl

TABLE 24
Hydrocodone plus hydromorphone pharmacokinetic parameters following oral
administration of hydrocodone bitartrate or YYFFI-HC at escalating doses.

| | Dose¹/Concentration (ng/ml) | | | | | | | |
|---|---|---|---|---|---|---|---|---|
| | 1 mg/kg | | 2 mg/kg | | 5 mg/kg | | 25 mg/kg | |
| Parameter | HC² | YYFFI-HC³ | HC² | YYFFI-HC³ | HC² | YYFFI-HC³ | HC² | YYFFI-HC³ |
| AUC | 53 | 34 | 59 | 61 | 312 | 62 | 569 | 193 |
| Percent HC⁴ | 100 | 64 | 100 | 103 | 100 | 20 | 100 | 34 |
| Cmax | 116 | 20.8 | 67.9 | 36.3 | 638 | 32.9 | 453 | 49.4 |
| Percent HC⁴ | 100 | 18 | 100 | 53 | 100 | 5 | 100 | 11 |

¹hydrocodone base content
²hydrocodone bitartrate
³YYFFI-HC HCl
⁴percent relative to parameter following administration of hydrocodone bitartrate

TABLE 25
Mean hydrocodone plus hydromorphone, hydrocodone, and
hydromorphone, concentrations following intravenous administration
of hydrocodone bitartrate or YYFFI-
HC at 1 mg/kg (hydrocodone base content).

| | Concentration (ng/ml) | | | | | |
|---|---|---|---|---|---|---|
| | HC + HM | | HC | | HM | |
| Hours | HC¹ | YYFFI-HC² | HC¹ | YYFFI-HC² | HC¹ | YYFFI-HC² |
| 0 | 0 | 0 | 0 | 0 | 0 | 0 |
| 0.1 | 208.9 | 22.6 | 42.97 | 8.75 | 251.9 | 31.3 |
| 0.5 | 83.7 | 13.5 | 16.09 | 1.44 | 99.8 | 14.9 |
| 1.0 | 38.4 | 13.0 | 3.65 | 0.92 | 42.1 | 13.9 |
| 2.0 | 12.4 | 13.1 | 1.77 | 0.41 | 14.2 | 13.5 |
| 4.0 | 2.9 | 8.5 | 0.70 | 0.33 | 3.6 | 8.8 |

¹hydrocodone bitartrate
²YYFFI-HC HCl

TABLE 26
Hydrocodone plus hydromorphone, hydrocodone, and hydromorphone
pharmacokinetic parameters following intravenous administration of
hydrocodone bitartrate or YYFFI-HC at 1 mg/kg
(hydrocodone base content).

| Pa- | Concentration (ng/ml) | | | | | |
|---|---|---|---|---|---|---|
| ram- | HC + HM | | HC | | HM | |
| eter | HC¹ | YYFFI-HC² | HC¹ | YYFFI-HC² | HC¹ | YYFFI-HC² |
| AUC | 140.0 | 50.0 | 24.10 | 4.50 | 164 | 54 |
| Per- | 100 | 36 | 100 | 19 | 100 | 33 |
| cent¹ | | | | | | |
| Cmax | 208.9 | 22.6 | 43.0 | 8.7 | 252 | 31.3 |
| Per- | 100 | 10.8 | 100 | 20.2 | 100 | 12.4 |
| cent¹ | | | | | | |

¹hydrocodone bitartrate
²YYFFI-HC HCl
³percent relative to parameter following administration of hydrocodone bitartrate

TABLE 27
Mean hydrocodone plus hydromorphone, hydrocodone, and
hydromorphone, concentrations following intranasal administration
of hydrocodone bitartrate or YYFFI-HC at 1 mg/kg.

| | Concentration (ng/ml) | | | | | |
|---|---|---|---|---|---|---|
| | HC + HM | | HC | | HM | |
| Minutes | HC¹ | YYFFI-HC² | HC¹ | YYFFI-HC² | HC¹ | YYFFI-HC² |
| 0 | 0 | 0 | 0 | 0 | 0 | 0 |
| 5 | 446 | 28 | 441 | 28 | 4.4 | bql³ |
| 15 | 553 | 27 | 543 | 27 | 10.6 | bql⁴ |
| 30 | 244 | 16 | 227 | 16 | 17.1 | bql⁵ |
| 60 | 103 | 21 | 96 | 21 | 7.2 | bql⁶ |

¹hydrocodone bitartrate
²YYFFI-HC HCl

TABLE 28
Hydrocodone plus hydromorphone, hydrocodone, and hydromorphone
pharmacokinetic parameters following intravenous administration of
hydrocodone bitartrate or YYFFI-HC at 1 mg/kg
(hydrocodone base content).

| Pa- | Concentration (ng/ml) | | | | | |
|---|---|---|---|---|---|---|
| ram- | HC + HM | | HC | | HM | |
| eter | HC¹ | YYFFI-HC² | HC¹ | YYFFI-HC² | HC¹ | YYFFI-HC² |
| AUC | 288.0 | 20.0 | 74.70 | 10.30 | 7.0 | NA |
| Per- | 100 | 6.9 | 100 | 13.8 | 100 | NA |
| cent³ | | | | | | |
| Cmax | 553.0 | 28.0 | 543.0 | 28.0 | 17 | NA |
| Per- | 100 | 5.1 | 100 | 5.2 | 100 | NA |
| cent³ | | | | | | |

¹hydrocodone bitartrate
²YYFFI-HC HCl
³percent relative to parameter following administration of hydrocodone bitartrate

Summary of in vivo testing of abuse resistant hydrocodone conjugates. In vivo testing of hydrocodone conjugates demonstrates for instance decreased intranasal analgesic response, decreased intravenous analgesic response, decreased subcutaneous analgesic response, decreased oral Cₘₐₓ, decreased intranasal bioavailability (AUC and Cₘₐₓ), and decreased intravenous bioavailability (AUC and Cₘₐₓ) of hydrocodone conjugates and is described in further detail below.

### Example 46

### Decreased Intranasal Analgesic Response to Hydrocodone Conjugates

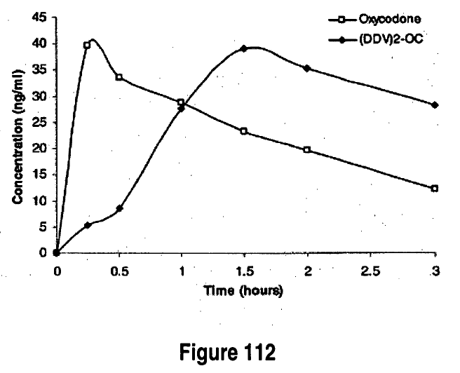

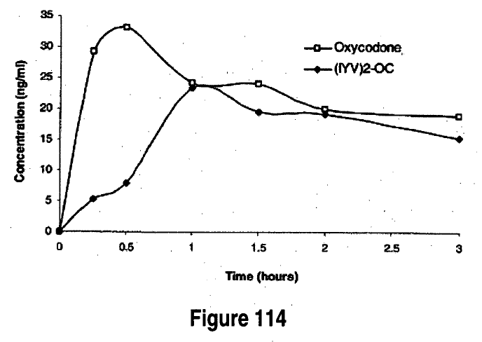

Male Sprague-Dawley rats were dosed by placing 0.02 ml of water containing hydrocodone conjugate or hydrocodone bitartrate into the nasal flares. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curves shown in FIGS. 112 and 114 indicate the decrease in analgesia produced by the hydrocodone conjugates as compared to an equimolar (hydrocodone base) dose of hydrocodone bitartrate. The analgesic response as determined by the hot plate test is a pharmacodynamic measurement of the pharmacological effect of hydrocodone. These examples illustrate that hydrocodone conjugates decrease the analgesic effect by the intranasal route of administration as compared to hydrodone bitartrate.

### Example 47

### Decreased Intravenous Analgesic Response to Hydrocodone Conjugates

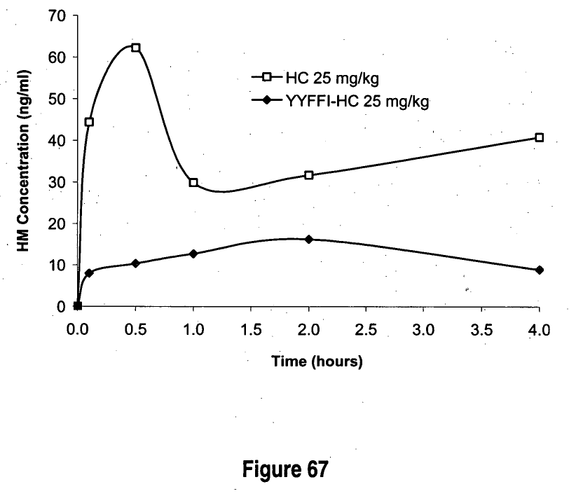

Male Sprague-Dawley rats were dosed by tail vein injection of 0.1 ml of water containing hydrocodone conjugates or hydrocodone bitartrate. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curve shown in FIG. 67 indicates the decrease in analgesia produced by a hydrocodone conjugate as compared to an equimolar (hydrocodone base) dose of hydrocodone bitartrate. The analgesic response as determined by the hot plate test is a pharmacodynamic measurement of the pharmacological effect of hydrocodone. This example illustrates that a hydrocodone conjugate decreased the analgesic effect by the intravenous route of administration as compared to hydrodone bitartrate.

### Example 48

### Decreased Subcutaneous Analgesic Response to Hydrocodone Conjugates

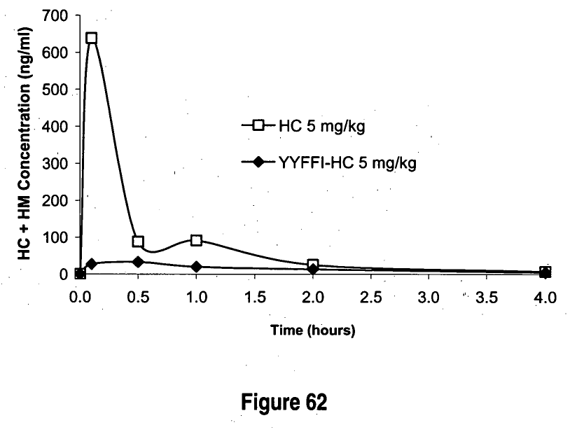

Male Sprague-Dawley rats were dosed by subcutaneous injection of 0.1 ml of water containing hydrocodone conjugates or hydrocodone bitartrate. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curve shown in FIG. 62 indicates the decrease in analgesia produced by a hydrocodone conjugate as compared to an equimolar (hydrocodone base) dose of hydrocodone bitartrate. The analgesic response as determined by the hot plate test is a pharmacodynamic measurement of the pharmacological effect of hydrocodone. This example illustrates that a hydrocodone conjugate decreased the analgesic effect by the subcutaneous route of administration as compared to hydrodone bitartrate.

### Example 49

### Decreased Oral Cₘₐₓ of Hydrocodone Conjugates

Male Sprague-Dawley rats were provided water ad libitum, fasted overnight and dosed by oral gavage with hydrocodone conjugates or hydrocodone bitartrate. All doses contained equivalent amounts of hydrocodone base. Plasma hydrocodone concentrations were measured by ELISA (Hydromorphone, 106619-1, Neogen, Corporation, Lexington, Ky.). The assay is specific for hydromorphone (the major hydrocodone metabolite, 100% reactive) and hydrocodone (62.5% reactive). The plasma concentration-time curves of various hydrocodone conjugates vs. hydrocodone bitratrate are shown in FIGS. 53, 76, 84, and 85. These examples illustrate that hydrocodone conjugates decrease the peak level (Cₘₐₓ) of hydrocodone plus hydromorphone as compared to that produced by equimolar (hydrocodone base) doses of hydrocodone bitartrate when given by the oral route of administration.

### Example 50

### Decreased Intranasal Bioavailability (AUC and Cₘₐₓ) Hydrocodone Conjugates

Male Sprague-Dawley rats were provided water ad libitum and doses were administered by placing 0.02 ml of water containing hydrocodone conjugates or hydrocodone bitartrate into the nasal flares. All doses contained equivalent amounts of hydrocodone base. Plasma hydrocodone concentrations were measured by ELISA (Hydromorphone, 106619-1, Neogen, Corporation, Lexington, Ky.). The assay is specific for hydromorphone (the major hydrocodone metabolite, 100% reactive) and hydrocodone (62.5% reactive). The plasma concentration-time curves of various hydrocodone conjugates vs. hydrocodone bitartrate are shown in FIGS. 55, 60, 64-66, 69-73, 75, 77-85. These examples illustrate that hydrocodone conjugates decrease the peak level (Cₘₐₓ) and total absorption (AUC) of hydrocodone plus hydromorphone as compared to those produced by equimolar (hydrocodone base) doses of hydrocodone bitartrate when given by the intranasal route of administration.

### Example 51

### Decreased Intravenous Bioavailability (AUC and Cₘₐₓ) Hydrocodone Conjugates

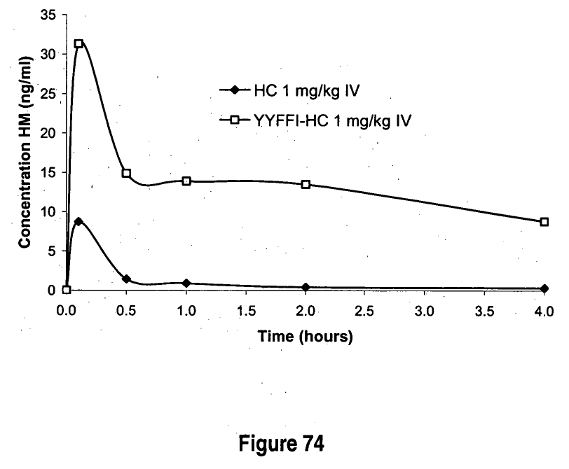

Male Sprague-Dawley rats were provided water ad libitum and doses were administered by intravenous tail vein injection of 0.1 ml of water containing hydrocodone conjugates or hydrocodone bitartrate. All doses contained equivalent amounts of d-amphetamine base. Plasma hydrocodone concentrations were measured by ELISA (Hydromorphone, 106619-1, Neogen, Corporation, Lexington, Ky.). The assay is specific for hydromorphone (the major hydrocodone metabolite, 100% reactive) and hydrocodone (62.5% reactive). The plasma concentration-time curves of a hydrocodone conjugate vs. hydrocodone bitartrate is shown in FIG. 74. This example illustrates that a dose of hydrocodone conjugate decreases the peak level (Cₘₐₓ) and total absorption (AUC) of hydrocodone plus hydromorphone as compared to those produced by an equimolar (hydrocodone base) dose of hydrocodone bitartrate when given by the intranasal route of administration.

### Examples 52 through 86

### Oxycodone

Examples 52 through 86 illustrate the compounds and compositions for reducing the potential for overdose and abuse while maintaining therapeutic value wherein the active agent oxycodone (OC) is covalently attached to a chemical moiety. The compound which is di-substituted at the 6 and 14 position of oxycodone is termed PPL(2)-OC.

Oral, intranasal, and intravenous bioavailability studies of oxycodone and oxycodone conjugates were conducted in male Sprague-Dawley rats. Doses of oxycodone hydrochloride and oxycodone conjugates containing equivalent amounts of oxycodone were administered in deionized water. Oral administration was in 0.5 ml by gavage needle. Intranasal doses were administered by placing 20 microliters into the nasal flares of rats anesthetized with isoflurane. Intravenous administration was in 0.1 ml by tail vein injection. Plasma was collected by retroorbital sinus puncture under isoflurane anesthesia. Oxycodone and oxymorphone (major active metabolite) concentrations were determined by LC/MS/MS.

The below examples are illustrative only and PPL(2)-OC is not meant to be limiting. As such, synthesis and attachment of oxycodone may be accomplished for instance view the following exemplary methods. Additionally, Examples 52 through 64 describe methods for attaching amino acid or various length peptides to oxycodone.

Oxycodone Synthetic Examples

### Example 52

### Synthesis of [Boc-X]₂-Oxycodone

To a solution of oxycodone free base (2.04 g, 6.47 mmol) in THF (˜35 ml) was added LiN(TMS)₂ (19.41 ml, 19.41 mmol) and stirred for ˜30 mins. To this was added solid Boc-X—OSu (X=amino acid, 21 mmol) at one time and the reaction mixture was stirred at room temperature overnight. The solution was neutralized with 1N HCl and the THF was removed under reduced pressure. The residue was diluted with EtOAc (200 mL), satd. NaHCO₃ (150 mL) was added and stirred for 1 h. EtOAc part was washed with NaHCO3 and brine. Dried over Na₂SO₄ and evaporated to dryness. Compound was obtained by purification over silica gel column (30% EtOAc/Hexane).

Deprotection of [Boc-X]₂-Oxycodone:

General method of deprotection: The above compound was reacted with 4N HCl/dioxane (25 mL/gm) at room temperature for 4 h. Solvent was evaporated and dried over vacuum to give X₂-Oxycodone.3HCl.

Examples:

1. (Val)₂-Oxycodone

2. (Ile)₂-Oxycodone

3. (Leu)₂-Oxycodone

4. (Lys)₂-Oxycodone

5. (Phe)₂-Oxycodone

6. (Glu)₂-Oxycodone

### Example 53

### Synthesis of [Boc-Z-Y—X]₂-Oxycodone [X, Y and Z are Amino Acids]

To a solution of X₂-Oxycodone.3HCl (1 mmol) in DMF (15-20 mL) were added NMM (10-12 eqv) and Boc-Z-Y—OSu (2.6 eqv). The reaction mixture was stirred at RT overnight. Solvent was evaporated under reduced pressure. To the residue was added satd. NaHCO₃ (˜30 mL) and stir for 1-2 h. The white/pale yellow residue was filtered, thoroughly washed with water and dried in the vacuum oven at room temperature.

Deprotection of [Boc-X—Y-Z]₂-Oxycodone:

Deprotection is same as general method mentioned above. For 100-200 mg of tripeptide derivative 10-15 ml 4N HCl/dioxane is used. Deprotection is done overnight to give [X—Y-Z]₂-Oxycodone.3HCl.

Deprotection of Tripeptide Derivatives Containing Threonine and Serine:

First the tripeptide derivatives are dissolved 95% TFA (5% water) and stirred for 4 h at room temperature. Solvent is evaporated, the residue is co-evaporated with toluene twice and dried over vacuum. 4N HCl/dioxane is added and stirred overnight. Residue was evaporated to dryness and dried over vacuum.

Examples:

1. (Glu-Asp-Val)₂-Oxycodone

2. (Ile-Tyr-Val)₂-Oxycodone

3. (Tyr-Pro-Val)₂-Oxycodone

4. (Gly-Leu-Val)₂-Oxycodone

5. (Phe-Val-Val)₂-Oxycodone

6. (Ser-Thr-Val)₂-Oxycodone

7. (Lys-Ser-Val)₂-Oxycodone

### Example 54

### Synthesis of [Boc-X]-O⁶-Oxycodone

To a solution of oxycodone (10 mmol) in THF (50 mL) was added LiN(TMS)₂ (10.5 mmol) at 0° C. After 20 mins was added Boc-X—OSu (11 mmol) and then the reaction mixture was stirred at room temperature overnight. The solution was cooled down to 0° C. and neutralized with 1N HCl. The organic solvent was evaporated and to the residue were added EtOAc (200 mL) and saturated aq. NaHCO₃ (150 mL) and stirred for 1 h. The EtOAc portion was washed with water, brine, dried over Na₂SO₄ and evaporated to dryness. The residue was purified over silica gel (70% EtOAc-Hexane) to give the title compound.

Deprotection of Boc-X—O⁶-Oxycodone:

A solution of [Boc-X]-Oxycodone in 4N HCl/dioxane (10 ml/mmol) was stirred at room temperature 4 h. Solvent was evaporated under reduced pressure and the residue was dried under vacuum to give X—O⁶-Oxycodone.2HCl.

Examples:

1. Val-Oxycodone

2. Ile-Oxycodone

3. Leu-Oxycodone

### Example 55

### Synthesis of Boc-Z-Y—X—O⁶-Oxycodone

To a solution of X—O⁶-Oxycodone.2HCl (1 mmol) in DMF were added NMM (10 mmol) and Boc-Z-Y—OSu (1.2 mmol). The reaction mixture was stirred at room temperature overnight. Solvent was evaporated to the residue was added saturated NaHCO₃ solution and stirred for 1 h. The precipitate was filtered, thoroughly washed with water and dried to give the title compound.

Deprotection of Boc-Z-Y—X—O⁶-Oxycodone:

Deprotection is same as general method mentioned above to give Z-Y—X—O⁶-Oxycodone.2HCl.

Examples:

1. Pro-Glu-Val-Oxycodone

2. Glu-Leu-Val-Oxycodone

3. Glu-Tyr-Val-Oxycodone

### Example 56

### Synthesis of Boc-X—O⁶-Oxycodone-O¹⁴—Ac

To a solution of [Boc-X]-O⁶-Oxycodone (1 mmol) in pyridine (15 mL) were added DMAP (75 mg), triethyl amine (1.5 mmol) and Ac₂O (8 mmol). The reaction mixture was heated at 65° C. for 3 days. The dark brown solution was cooled down to room temperature and MeOH (5 mL) was added and stirred for 1 h. The solvent was evaporated, co-evaporated with toluene. The residue was taken in EtOAc (50 mL), washed with satd. NaHCO₃, brine, dried over Na₂SO₄ and evaporated to dryness. The residue was purified over silila gel to give the title compound.

### Example 57

### Synthesis of Boc-X—O⁶-Oxycodone-O¹⁴—CO₂Et

To a solution of [Boc-X]-O⁶-Oxycodone (1 mmol) in THF (10 mL) was added LiN(TMS)₂ (1.05 mmol) at 0° C. After 20 mins, ethyl chloroformate (1.1 mmol) was added and reaction mixture was slowly brought to room temperature and stirred at room temperature for 1 h. The solution was poured into 2% aqueous acetic acid (ice cold) and extracted with EtOAc. The EtOAc part was washed with water, aq. NaHCO₃, brine, dried over Na₂SO₄ and evaporated to dryness. The residue was purified over silica gel to give the title compound.

Deprotection of Boc-X—O⁶-Oxycodone-O¹⁴—R(R═Ac, CO₂Et):

Deprotection is same as general method mentioned above to give X—O⁶-Oxycodone-O¹⁴—R.2HCl (R═Ac, CO₂Et).

Examples:

1. (Val)-Oxycodone-(CO₂Et)

2. (Val)-Oxycodone-(OAc)

### Example 58

### Synthesis of Boc-Z-Y—X—O⁶-Oxycodone-O¹⁴—R(R═Ac, CO₂Et)

To a solution of X—O⁶-Oxycodone-O¹⁴—R.2HCl (1 mmol, R═Ac, CO₂Et) in DMF were added NMM (10 mmol) and Boc-Z-Y—OSu (1.2 mmol). The reaction mixture was stirred at room temperature overnight. Solvent was evaporated to the residue was added saturated NaHCO₃ solution and stirred for 1 h. The precipitate was filtered, thoroughly washed with water and dried to give the title compound.

Deprotection of Boc-Z-Y—X—O⁶-Oxycodone-O¹⁴—R(R═Ac, CO₂Et):

Deprotection is same as general method mentioned above. Deprotection is done overnight to give Z-Y—X—O⁶-Oxycodone-O¹⁴—R.2HCl.

Examples:

1. (Ile-Tyr-Val)-Oxycodone-(CO₂Et)

2. (Ile-Tyr-Val)-Oxycodone-(OAc)

### Example 59

### Synthesis of Boc-X—O⁶-Oxycodone-O¹⁴—Y-Boc

To a solution of Boc-X-Oxycodone (1 mmol) in THF (10 mL) was added LiN(TMS)₂ (1.1 mmol) at 0° C. and the solution was stirred for 30 mins then Boc-Y—OSu (1.25 mmol) was added. The reaction mixture was stirred at room temperature overnight. The solution was cooled down to 0° C., neutralized with 1N HCl and the organic part was evaporated. To the residue were added EtOAc (50 mL) and satd. NaHCO₃ (50 ml), stirred for 1 h. The organic part was washed with water, brine, dried over Na₂SO₄ and evaporated to dryness. The residue was purified over silica gel to give the title compound.

Deprotection of Boc-X—O⁶-Oxycodone-O¹⁴—Y-Boc:

Boc-X—O⁶-Oxycodone-O¹⁴—Y-Boc was deprotected following the general method for deprotection mentioned above to give X—O⁶-Oxycodone-O¹⁴—Y.3HCl.

Example:

Val-Oxycodone-Gly

### Example 60

### Synthesis of Boc-A-B—X—O⁶-Oxycodone-O¹⁴—Y—B-A-Boc (A,B,X,Y=Amino Acids)

To a solution of X—O⁶-Oxycodone-O¹⁴—Y.3HCl (1 mmol) and NMM (10 mmol) in DMF (10 mL) was added Boc-A-B—OSu (2.5 mmol) and the reaction mixture was stirred at room temperature overnight. Solvent was evaporated under reduced pressure and to the residue satd. NaHCO₃ (15 mL) was added and stirred for 1 h. The precipitate was filtered off and the residue was washed thoroughly with water and dried.

Deprotection of Boc-A-B—X—O⁶-Oxycodone-O¹⁴—Y—B-A-Boc:

Deprotection is same as general method mentioned above. Deprotection is done overnight to give A-B—X—O⁶-Oxycodone-O¹⁴—Y—B-A.3HCl.

Examples:

1. (Ile-Tyr-Val)-Oxycodone-(Gly-Tyr-Ile)

2. (Leu-Tyr-Val)-Oxycodone-(Gly-Tyr-Leu)

### Example 61

### Synthesis of Boc-X—O⁶-Oxycodone-O¹⁴—Y-Cbz

To a solution of Boc-X-Oxycodone (1 mmol) in THF (10 mL) was added LiN(TMS)₂ (1.1 mmol) at 0° C. and the solution was stirred for 30 mins then Cbz-Y—OSu (1.25 mmol) was added. The reaction mixture was stirred at room temperature overnight. The solution was cooled down to 0° C., neutralized with 1N HCl and the organic part was evaporated. To the residue were added EtOAc (50 mL) and satd. NaHCO₃ (50 ml), stirred for 1 h. The organic part was washed with water, brine, dried over Na₂SO₄ and evaporated to dryness. The residue was purified over silica gel to give the title compound.

Deprotection of Boc-X—O⁶-Oxycodone-O¹⁴—Y-Cbz.2HCl:

Boc-X—O⁶-Oxycodone-O¹⁴—Y-Cbz was deprotected following the general method for deprotection mentioned above to give X—O⁶-Oxycodone-O¹⁴—Y-Cbz.2HCl.

### Example 62

### Synthesis of Boc-A-B—X—O⁶-Oxycodone-O¹⁴—Y-Cbz

To a solution of X—O⁶-Oxycodone-O¹⁴—Y-Cbz.2HCl (1 mmol) and NMM (10 mmol) in DMF (10 mL) was added Boc-A-B—OSu (1.1 mmol) and the reaction mixture was stirred at room temperature overnight. Solvent was evaporated under reduced pressure and to the residue satd. NaHCO₃ (20 mL) was added and stirred vigorously for 2-3 h. The precipitate was filtered off and the residue was washed thoroughly with water and dried.

### Example 63

### Synthesis of Boc-A-B—X—O⁶-Oxycodone-O¹⁴—Y—NH2

To a suspension of Boc-A-B—X—O⁶-Oxycodone-O¹⁴—Y-Cbz and Pd/C (25 Wt %) in EtOH (20 ml/gm) and cyclohexene (10 ml/gm) was heated under reflux for 30 mins. The reaction mixture was cooled down to room temperature and filtered. The filtrate was evaporated to dryness to give the title compound.

### Example 64

### Synthesis of Boc-A-B—X—O⁶-Oxycodone-O¹⁴—Y—C-D-Boc (A,B,C,D,X,Y=Amino Acids)

To a solution of Boc-A-B—X—O⁶-Oxycodone-O¹⁴—Y—NH₂ (1 mmol) in DMF (10 mL) were added NMM (5 mmol) and Boc-D-C—OSu (1.1 mmol) and the reaction mixture was stirred at room temperature overnight. Solvent was evaporated under reduced pressure and to the residue satd. NaHCO₃ was added and stirred for 1 h. The white precipitate was filtered, washed with water and dried.

Deprotection of Boc-A-B—X—O⁶-Oxycodone-O¹⁴—Y—C-D-Boc:

Deprotection is same as general method mentioned above. Deprotection is done overnight to give A-B—X—O⁶-Oxycodone-O¹⁴—Y—C-D.3HCl.

Examples:

1. (Ile-Tyr-Val)-Oxycodone-(Val-Glu-Gly)

2. (Leu-Tyr-Val)-Oxycodone-(Val-Glu-Gly)

Mono-Substituted Single Amino Acids (Enol Ester)

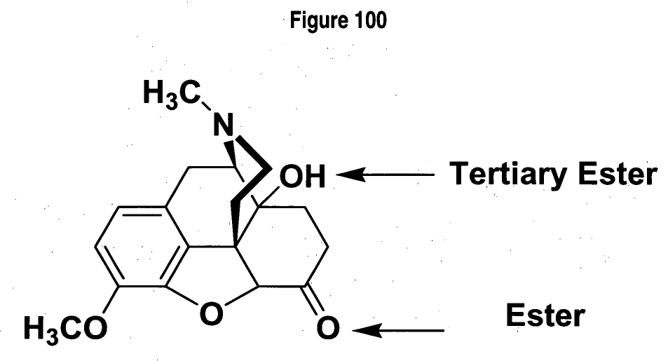

FIG. 100 depicts oxycodone.

### Example 65

### Phe-Oxycodone

To a solution of oxycodone-freebase (1.0 eq) in tetrahydrofuran (THF) (10 ml/mmol) was added LiN(TMS)₂ (3.5 eq). After 5 minutes, Boc-Phe-OSu (3.5 eq) was added. The reaction was stirred at ambient temperatures for 18 hours, quenched with water and solvents removed. Crude protected product was purified using reverse-phase HPLC. Deprotection occurred with 4N HCl in dioxane (20 ml/mmol) to obtain Phe-Oxycodone.

### Example 66

### Synthesis of Ile-Oxycodone

Ile-Oxycodone was prepared in a similar manner to Example 65 except Boc-Ile-OSu was used as the amino acid starting material.

Mono-Substituted Tripeptides (Enol Ester)

### Example 67

### Pro₂-Leu-Oxycodone

To a solution of Leu-Oxycodone (1.0 eq) in dimethylformamide (10 ml/0.1 mmol) was added 4-methylmorpholine (10 eq) and Boc-Pro-Pro-OSu (2 eq). The reaction was stirred at ambient temperatures for 18 hours, quenched with water, and solvents removed. Crude protected product was purified using reverse phase HPLC. Deprotection occurred using 4N HCl in dioxane (20 ml/mmol) to obtain Pro₂-Leu-Oxycodone.

### Example 68

### Synthesis of Pro₂-Ile-Oxycodone

Pro₂-Ile-Oxycodone was prepared in a similar manner to Example 67 except Ile-Oxycodone was used as the conjugated starting material.

### Example 69

### Oxycodone Disubstituted Tripeptides

General Synthetic Procedure

Synthesis of [Boc-Val]₂-OC:

To a solution of OC (2.04 g, 6.47 mmol) in tetrahydrofuran (THF) (˜35 ml) was added LiN(TMS)₂ (19.41 ml, 19.41 mmol) and stirred for ˜30 mins. To this was added solid Boc-Val-OSu (6.72 g, 21 mmol) at one time and the reaction mixture was stirred at room temperature overnight. The solution was neutralized with 1N HCl and the THF was removed under reduced pressure. The residue was diluted with ethyl acetate (EtOAc) (200 mL), satd. NaHCO₃ (150 mL) was added and stirred for 1 h. EtOAc part was washed with NaHCO₃ and brine. Dried over Na₂SO₄ and evaporated to dryness. Crude product was purified with either silica gel column. (30% EtOAc/Hexane).

Deprotection: For the deprotection of 2.5 g of [Boc-Val]₂-OC, 75-80 mL of 4N HCl/dioxane was used. Reaction was complete within 3-4 hours. Evaporate dioxane and dry over vacuum at lease for 24 h.

Coupling: To a solution of Val₂-OC.3HCl (250 mg, 0.4 mmol) in DMF (10-12 ml) were added NMM (10-12 eqv) and Boc-X—Y—OSu (2.6 eqv). The reaction mixture was stirred at RT overnight. Solvents were evaporated under reduced pressure. To the residue was added satd. NaHCO₃ (˜30 mL) and stirred for 1 h. The white/pale yellow residue was filtered, thoroughly washed with water and dried in the vacuum oven at RT.

Deprotection: Deprotection was same as above method. For 100-200 mg of tripeptide derivative 10-15 ml 4N HCl/dioxane was used. Deprotection lasts 18 hours.

Deprotection of tripeptide derivatives containing Threonine and Serine: Tripeptide derivatives were dissolved in 95% TFA (5% water) and stirred for 4 h at room temperature. Solvent was evaporated and the residue was co-evaporated with toluene twice and dried over vacuum. 4N HCl/dioxane was added and stirred overnight. Product was evaporated to dryness and dried over vacuum

### Example 70

### Oxycodone Branched Amino Acid Chains

General Synthesis

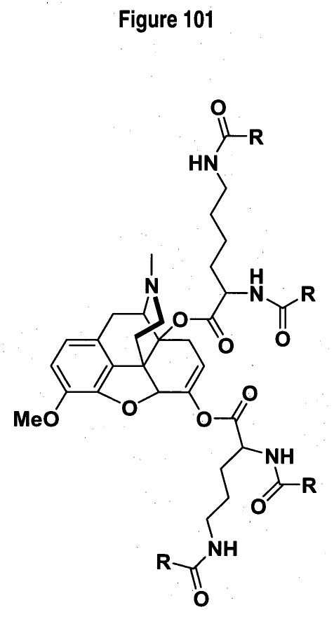

FIG. 101 depicts oxycodone with lysine branched peptides.

### Example 71

### (Lys)₂-Oxycodone

Method was similar to other single amino acid derivatives except Boc-Lys(Boc)-OSu was used as the amino acid starting material.

### Example 72

### XX-Lys(XX)-Oxycodone

To a solution of (Lys)₂-Oxycodone (1.0 eq) in dimethylformamide (1 ml/mmol) was added 4-methylmorpholine (5.5 eq) followed by Boc-XX₂—OSu (4.1). Reaction was stirred at ambient temperature for 24 hours. Solvents were removed and crude product was purified by reverse phase HPLC.

### Example 73

### Synthesis of [Gly₂-Lys(-Gly₂)]₂-Oxycodone

[Gly₂-Lys(-Gly₂)]₂-Oxycodone was prepared in a manner similar to Example 72 except Boc-Gly₂-OSu was used as the amino acid starting material.

### Example 74

### Oxycodone D-amino Acids

General Synthesis

Disubstituted D-amino acid tripeptides were prepared in a manner similar to disubstituted tripeptide conjugates except the amino acid starting material used the unnatural D-amino acids.

[(1)-Lys-(d)-Lys-Leu]₂-Oxycodone

To a solution of (Leu)₂-Oxycodone (1.0 eq) in dimethylformamide (1 ml/mmol) was added 4-methylmorpholine (10 eq) followed by Boc-(1)-Lys(Boc)-(d)-Lys(Boc)-OSu (3 eq). Reaction was stirred at ambient temperature for 24 hours. Solvents were removed and crude product was purified by reverse phase HPLC.

### Example 75

### Synthetic Amino Acids

Synthesis of [Boc-Z]₂-OC [where Z can equal cyclohexylalanine (Cha), dipropylglycine (Dpg), tert-Leucine (Tle) or any other synthetic amino acid]

To a solution of OC (6.47 mmol) in THF was added LiN(TMS)₂ (19.41 mmol) and stirred for ˜30 mins. To this was added solid Boc-Z-OSu (21 mmol) at one time and the reaction mixture was stirred at room temperature overnight. The solution was neutralized with 1N HCl and the THF was removed under reduced pressure. The residue was diluted with ethyl acetate (EtOAc), satd. NaHCO₃ was added and stirred for 1 h. EtOAc part was washed with NaHCO₃ and brine. Dried over Na₂SO₄ and evaporated to dryness. Crude product was purified with either silica gel column. (30% EtOAc/Hexane).

### Example 76

### Non-Standard Amino Acids (Naturally Occurring, Not the Standard 20)

Synthesis of [Boc-N]₂—OC [where N can equal norleucine (Nle), homophenylalanine (hPhe) or any other non-standard amino acid]

To a solution of OC (6.47 mmol) in THF was added LiN(TMS)₂ (19.41 mmol) and stirred for ˜30 mins. To this was added solid Boc-N—OSu (21 mmol) at one time and the reaction mixture was stirred at room temperature overnight. The solution was neutralized with 1N HCl and the THF was removed under reduced pressure. The residue was diluted with ethyl acetate (EtOAc), satd. NaHCO₃ was added and stirred for 1 h. EtOAc part was washed with NaHCO₃ and brine. Dried over Na₂SO₄ and evaporated to dryness. Crude product was purified with either silica gel column. (30% EtOAc/Hexane).

Other Oxycodone Conjugates

### Example 77

### Glycopeptides

Using galactose and a number of tripeptides, glycopeptides will be produced.

Initial Glycopeptides to be Produced

1. (Gal-Gly₂-Ile)₂-OC

2. (Gal-Pro₂-Ile)₂-OC

3. (Gal-Gly₂-Leu)₂-OC

4. (Gal-Pro₂-Leu)₂-OC

### Example 78

### Glycosylation of Oxycodone

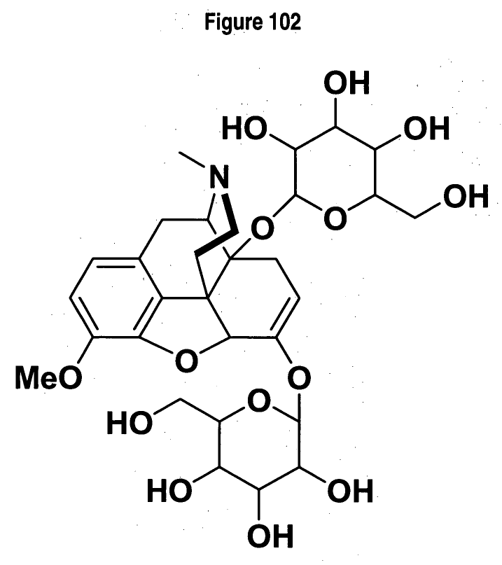

FIG. 102 depicts a glycosylated oxycodone.

A glycosylation reaction of Oxycodone with a carbohydrate will be attempted. The linkage produced would essentially be an enol ether which are difficult to cleave chemically yet glycosidic bonds are commonly broken down in vivo. Either site or both may be conjugated.

### Example 79

### Formation of an Enol Ether with Serine

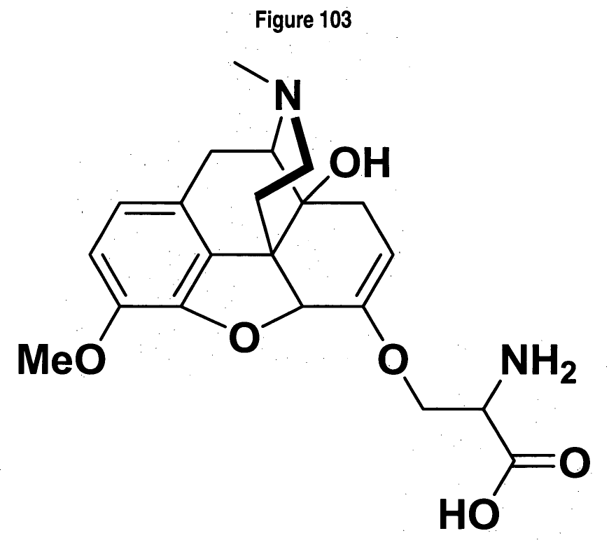

FIG. 103 depicts formation of an enol ether with serine.

Using serine and OC, an enol ether conjugate will be produced. This conjugate would be stable to most hydrolysis conditions. Only the enol ether would be formed in this reaction.

### Example 80

### Vitamins

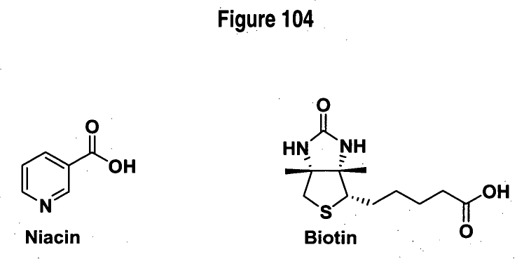

FIG. 104 depicts niacin and biotin.

Vitamins can be used to cap or further functionalize the peptide chain. Niacin and biotin will be conjugated to four different dipeptides.

Conjugates to Prepare

1. (Nia-Gly₂-Ile)₂-OC

2. (Nia-Gly₂-Leu)₂-OC

3. (Bio-Gly₂-Ile)₂-OC

4. (Bio-Gly₂-Leu)₂-OC

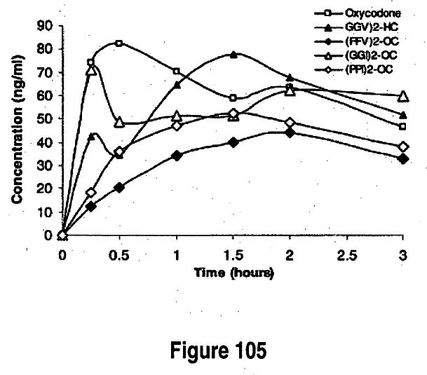

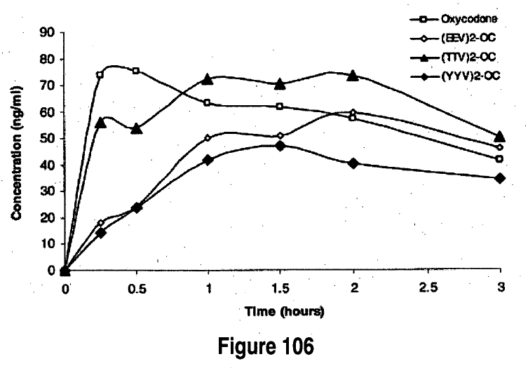

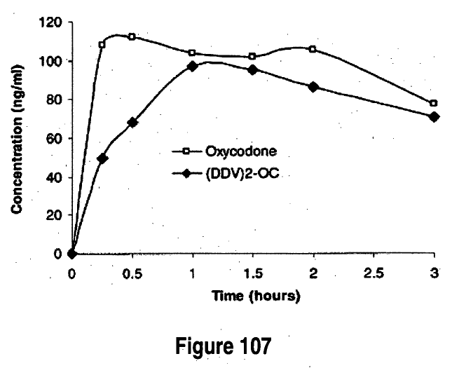

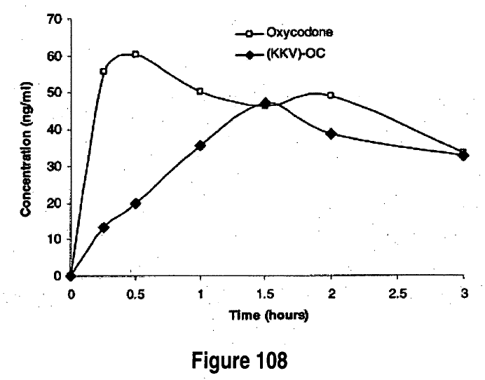

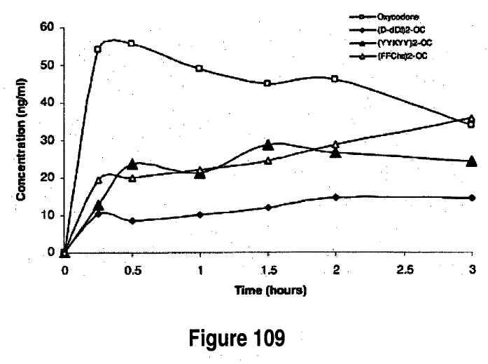

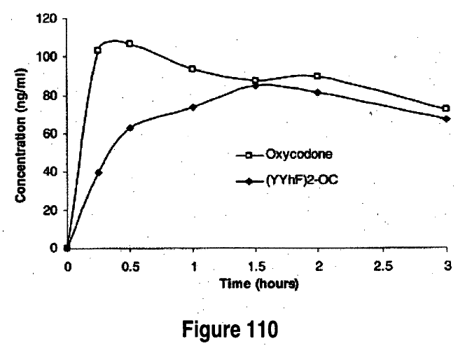

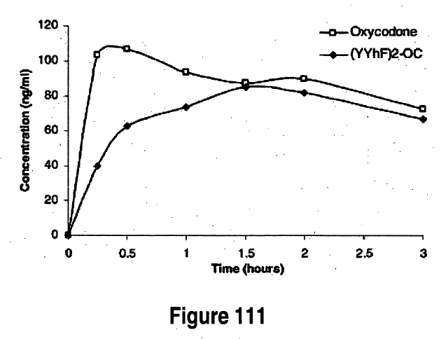

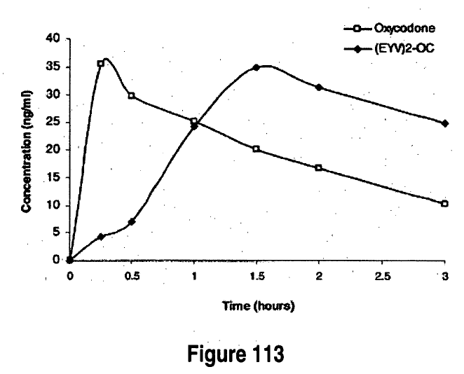

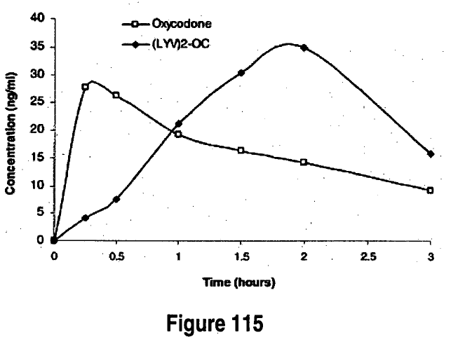

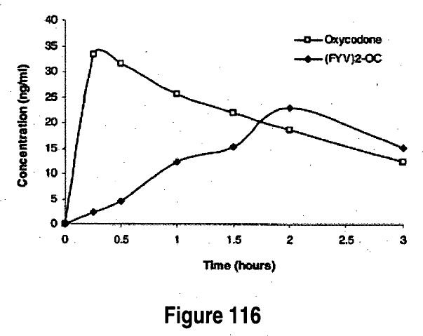

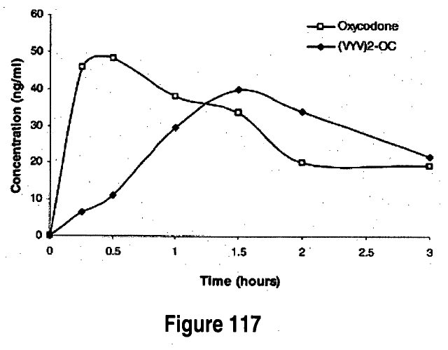

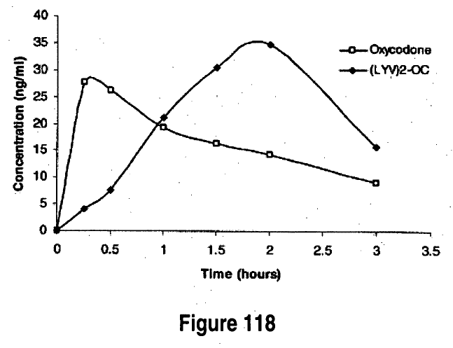

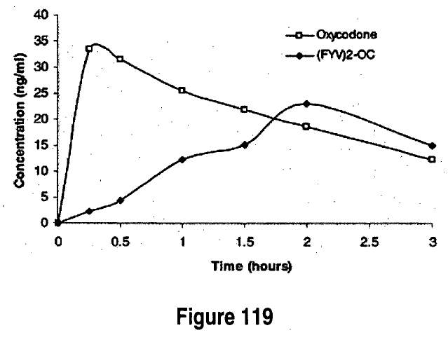

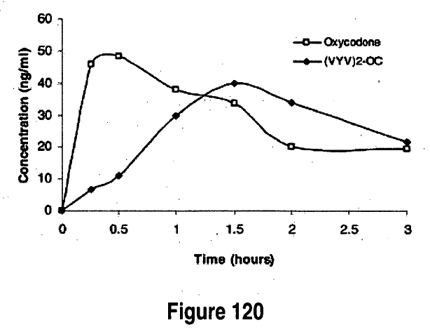

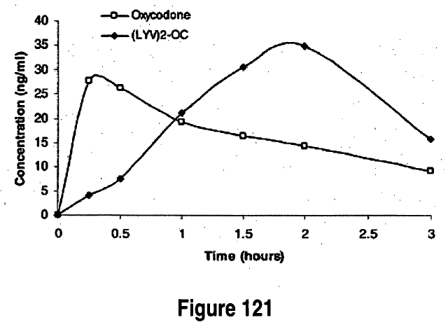

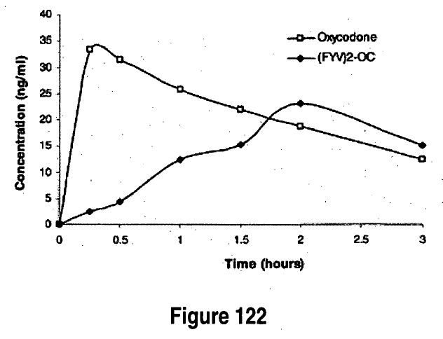

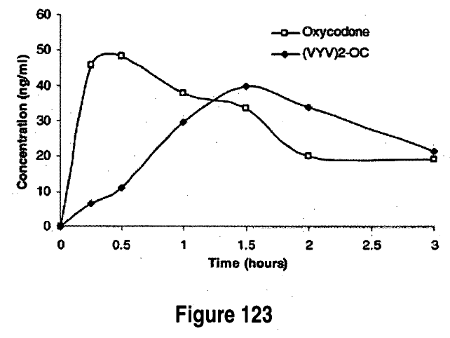

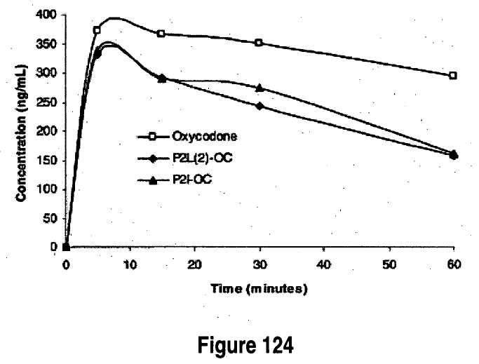

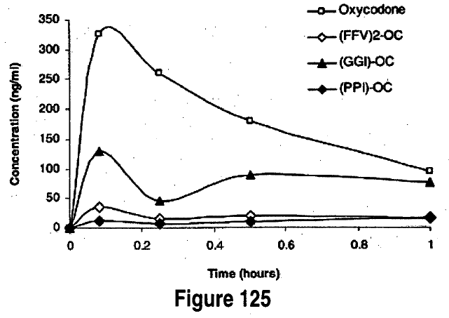

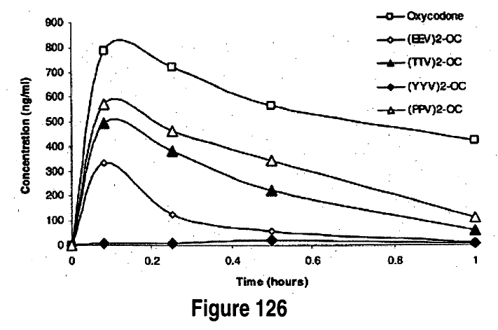

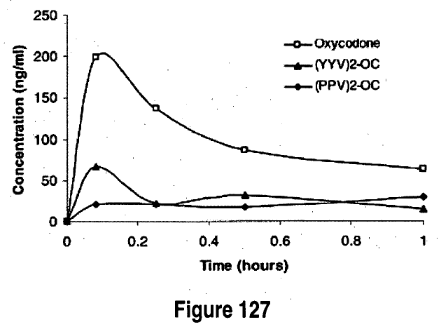

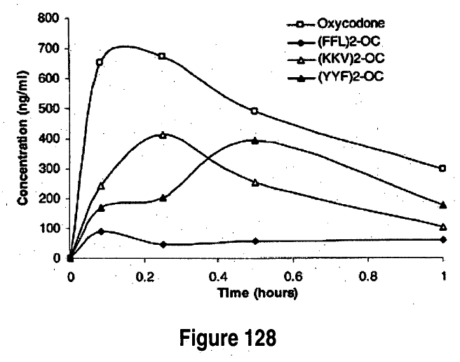

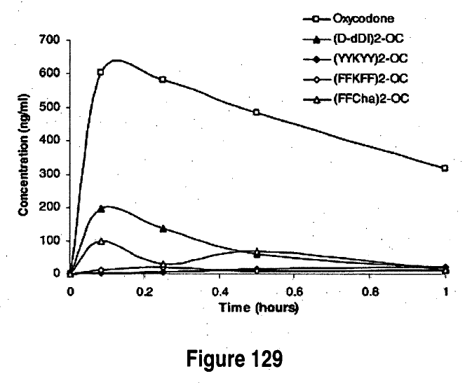

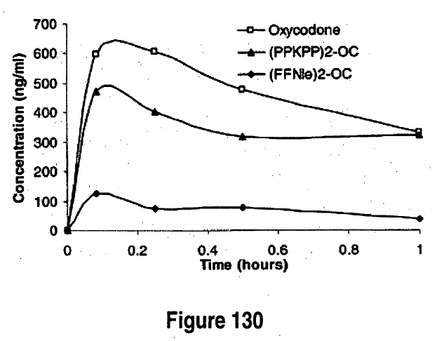

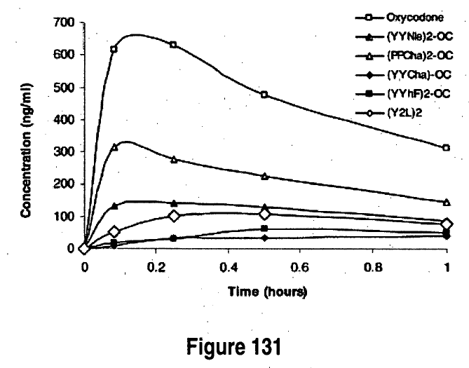

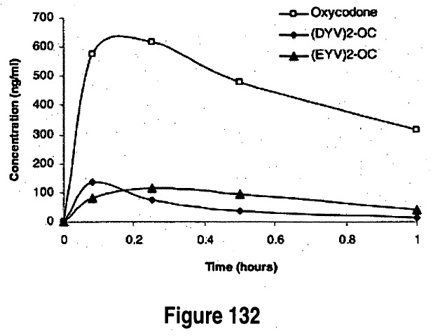

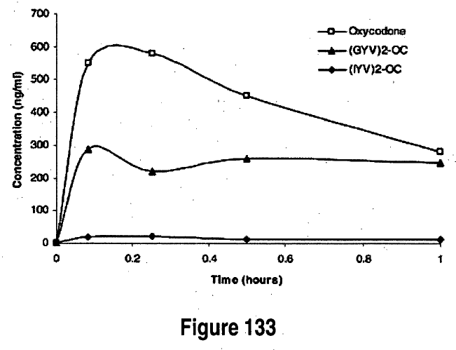

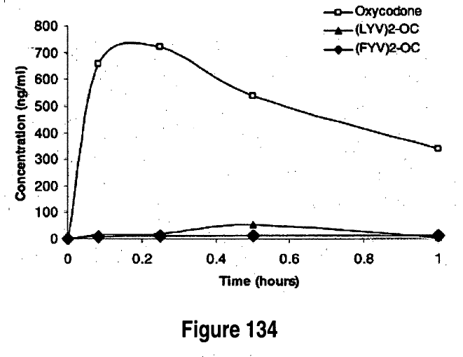

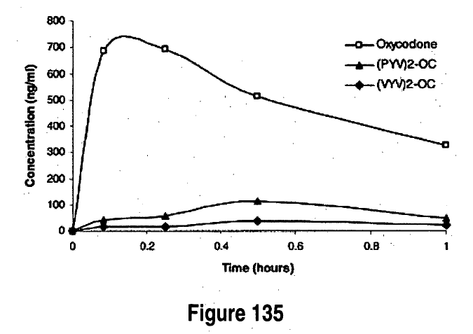

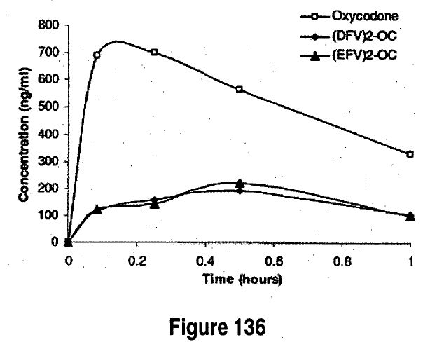

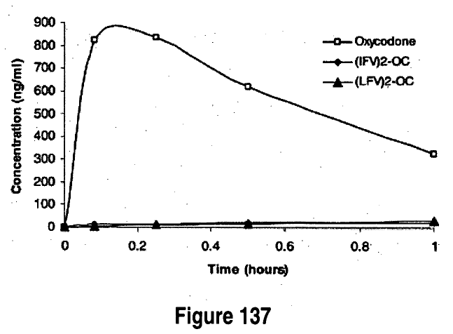

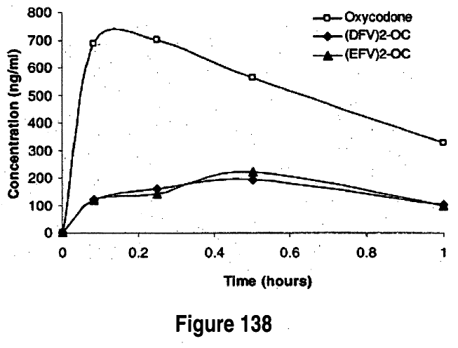

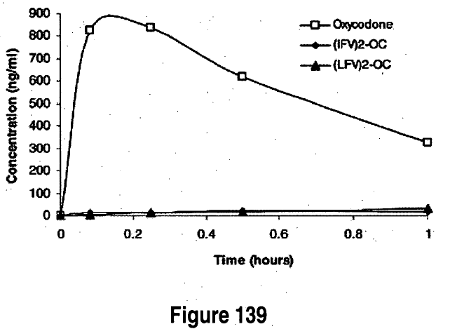

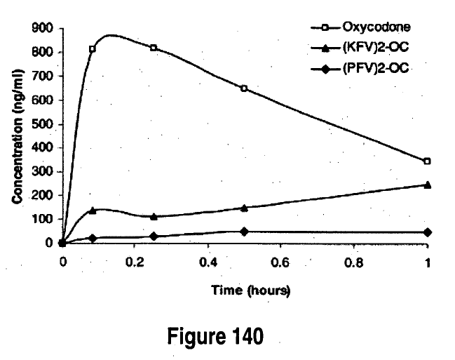

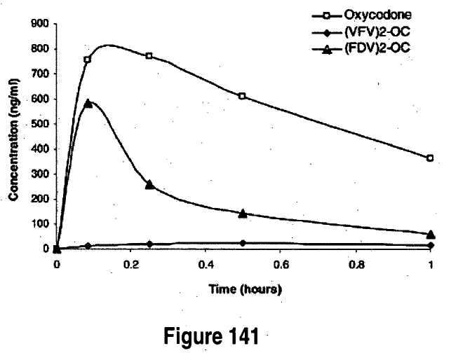

FIGS. 105-141 demonstrate plasma levels of oxycodone measured by ELISA.

### Example 81

### Decreased Oral Cₘₐₓ of Oxycodone Conjugates

Male Sprague-Dawley rats were provided water ad libitum, fasted overnight and dosed by oral gavage with oxycodone conjugates or oxycodone HCl. All doses contained equivalent amounts of oxycodone base. Plasma oxycodone concentrations were measured by ELISA (Oxymorphone, 102919, Neogen, Corporation, Lexington, Ky.). The assay is specific for oxymorphone (the major oxycodone metabolite) and oxycodone. Plasma concentration-time curves are shown in FIGS. 156-174. These examples illustrate that doses of oxycodone conjugates decrease the peak level (Cₘₐₓ) of oxycodone plus oxymorphone as compared to that produced by equimolar (oxycodone base) doses of oxycodone HCl when given by the oral route of administration.

### Example 82

### Oral Bioavailability of a Peptide-Oxycodone Conjugates at a Dose (2.5 mg/kg) Approximating a Therapeutic Human Dose

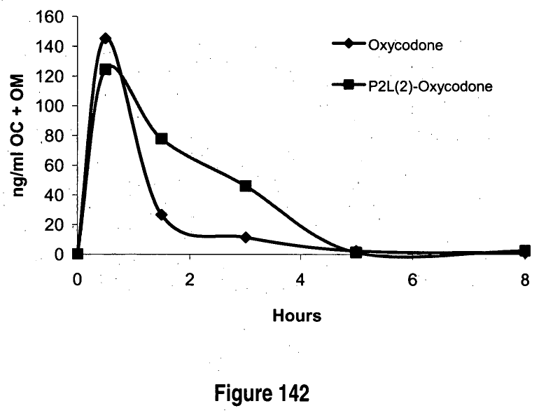

This example illustrates that when the peptide PPL (Table 29, FIG. 142) is conjugated (disubstituted at the 6 and 14 positions) to the active agent oxyocodone oral bioavailability is maintained as compared to an equimolar oxyocodone dose when the dose administered is 1 mg/kg. This dose is the equivalent of a human dose of 25 to 35 mg for an individual weighing 70 kg (148 lbs) according to Chou et al.

TABLE 29
Oral Pharmacokinetics of Oxycodone vs. P2L₍₂₎-OC (2.5 mg/kg dose).

| | Hours | | | | | AUC (ng/ml h) | Percent | Cmax | Percent |
|---|---|---|---|---|---|---|---|---|---|
| Drug | 0.5 | 1.5 | 3 | 5 | 8 | 0-8 h | OC | ng/ml | OC |
| Oxycodone Bitartrate | 145 | 27 | 11 | 2 | 1 | 168 | 100 | 145 | 100 |
| PPL(2)-OC | 124 | 78 | 46 | 1 | 3 | 278 | 165 | 124 | 86 |

oxycodone plus oxymorphone

### Example 83

### Bioavailability of P2L₍₂₎-Oxycodone by the Intranasal Route

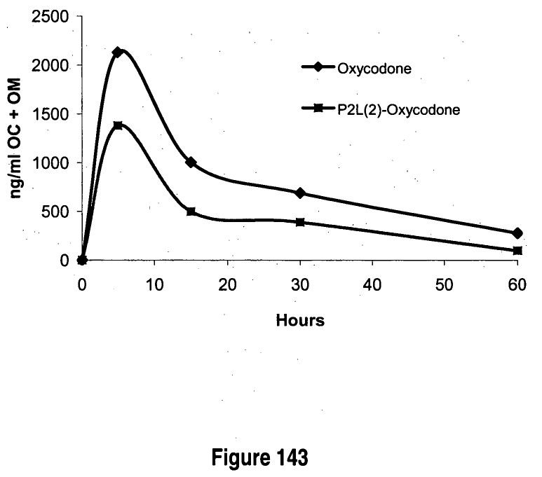

This example illustrates that when PPL(2) is conjugated to the active agent oxycodone the bioavailability by the intranasal route is substantially decreased thereby diminishing the possibility of overdose (Table 30, FIG. 143).

TABLE 30
Intranasal Pharmacokinetics of Oxyocodone vs. P2L₍₂₎-OC (1 mg/kg dose).

| | Minutes | | | | AUC (ng/ml h) | Percent | Cmax | Percent |
|---|---|---|---|---|---|---|---|---|
| Drug | 5 | 15 | 30 | 60 | 0-1 h | OC | ng/ml | OC |
| Oxycodone Bitartrate | 2128 | 1003 | 688 | 278 | 428 | 100 | 2128 | 100 |
| PPL(2)-OC | 1380 | 499 | 390 | 98 | 261 | 61 | 1380 | 65 |

oxycodone plus oxymorphone

### Example 84

### Bioavailability of P2L₍₂₎-Oxycodone by the Intravenous Route

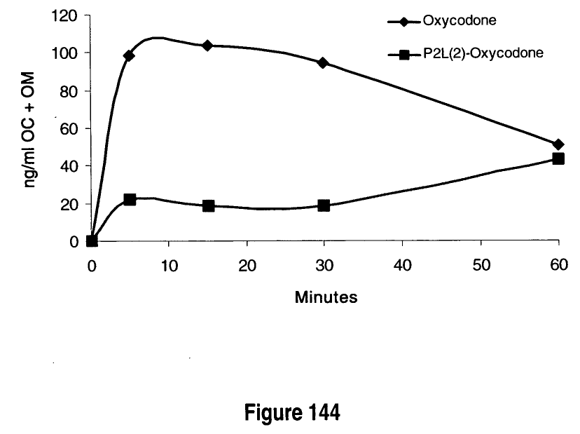

This example illustrates that when P2L₍₂₎ is conjugated to the active agent oxycodone the bioavailability by the intravenous route is substantially decreased thereby diminishing the possibility of overdose (Table 31, FIG. 144).

TABLE 31
Intravenous Pharmacokinetics of Oxyocodone
vs. P2L₍₂₎-OC (1 mg/kg dose).

| | | | | | AUC | | | |
|---|---|---|---|---|---|---|---|---|
| | Minutes | | | | (ng/ml h) | Percent | Cmax | Percent |
| Drug | 5 | 15 | 30 | 60 | 0-1 h | OC | ng/ml | OC |
| Oxycodone | 99 | 104 | 94 | 51 | 82 | 100 | 99 | 100 |
| Bitartrate | | | | | | | | |
| PPL(2)-OC | 22 | 19 | 19 | 43 | 24 | 29 | 43 | 43 |

oxycodone plus oxymorphone

Summary of In Vivo Testing of Abuse Resistant Oxycodone Conjugates.

In vivo testing of oxycodone conjugates demonstrates for instance decreased oral Cₘₐₓ, decreased intranasal bioavailability (AUC and Cₘₐₓ), and decreased intravenous bioavailability (AUC and Cₘₐₓ) and is described in further detail below.

### Example 85

### Decreased Intranasal Bioavailability (AUC and Cₘₐₓ) of Oxycodone Conjugates

Male Sprague-Dawley rats were provided water ad libitum and doses were administered by placing 0.02 ml of water containing oxycodone conjugates or oxycodone bitartrate into the nasal flares. All doses contained equivalent amounts of oxycodone base. Plasma oxycodone concentrations were measured by ELISA (Oxymorphone, 102919, Neogen, Corporation, Lexington, Ky.). The assay is specific for oxymorphone (the major oxycodone metabolite) and oxycodone. Plasma concentration-time curves of various oxycodone conjugates vs. oxycodone HCl are shown in FIGS. 175-192. These examples illustrate that oxycodone conjugates decrease the peak level (Cₘₐₓ) and total absorption (AUC) of oxycodone plus oxymorphone as compared to those produced by equimolar (oxycodone base) doses of oxycodone HCl when given by the intranasal route of administration.

### Example 86

### Decreased Intravenous Bioavailability (AUC and Cₘₐₓ) of Oxycodone Conjugates

Male Sprague-Dawley rats were provided water ad libitum and doses were administered by intravenous tail vein injection of 0.1 ml of water containing oxycodone conjugates or oxycodone HCl. All doses contained equivalent amounts of oxycodone base. Plasma oxycodone concentrations were measured by ELISA (Oxymorphone, 102919, Neogen, Corporation, Lexington, Ky.). The assay is specific for oxymorphone (the major oxycodone metabolite) and oxycodone. Plasma concentration-time curves of an oxycodone conjugate vs. oxycodone HCl is shown in FIG. 195. This example illustrates that an oxycodone conjugate decreases the peak level (Cₘₐₓ) and total absorption (AUC) of oxycodone plus oxymorphone as compared to those produced by an equimolar (oxycodone base) dose of oxycodone HCl when given by the intravenous route of administration.

| | oral 2 mg/kg | | intranasal 2 mg/kg | |
|---|---|---|---|---|
| | % AUC | % Cmax | % AUC | % Cmax |
| [Gly-Glu-Val]₂-OC | 93 | 61 | 29 | 48 |
| [Pro-Glu-Val]₂-OC | 90 | 82 | 34 | 46 |
| [Glu-Pro-Val]₂-OC | 142 | 134 | 56 | 65 |
| [Ser-Gly-Val]₂-OC | 90 | 92 | 64 | 73 |
| [Glu-Tyr-Val]₂-OC | 115 | 103 | 18 | 20 |
| [Gly-Tyr-Val]₂-OC | 92 | 99 | 56 | 54 |
| [Ile-Tyr-Val]₂-OC | 71 | 82 | 3 | 4 |
| [Leu-Tyr-Val]₂-OC | 131 | 120 | 4 | 5 |

OC = Oxycodone

Collectively, Examples 1 through 86 illustrate the application of the invention for reducing the overdose potential of narcotic analgesics. These examples establish that an active agent can be covalently modified by attachment of a chemical moiety in a manner that maintains therapeutic value over a normal dosing range, while substantially decreasing if not eliminating the possibility of overdose by oral, intranasal, or intravenous routes of administration with the active agent.

Oxycodone and acetaminophen are used together in the treatment of pain. The composition of the invention comprises oxycodone and acetaminophen covalently attached to a peptide.

Hydromorphone is a known pharmaceutical agent that is used in the treatment of cough and pain. The composition of the invention comprises hydromorphone covalently attached to a peptide. In the present invention, hydromorphone is covalently attached to the peptide via the hydroxyl group

Oxymorphone is a known pharmaceutical agent that is used in the treatment of pain. The composition of the invention comprises oxymorphone covalently attached to a peptide.

In the present invention, oxymorphone is covalently attached to the peptide via hydroxyl group.

Codeine is a known pharmaceutical agent that is used in the treatment of pain. The composition of the invention comprises codeine covalently attached to a peptide. In the present invention, codeine is covalently attached to the peptide via the hydroxyl group. Codeine and guaifenesin is a known pharmaceutical agent that is used in the treatment of coughs. The composition of the invention comprises codeine and guaifenesin covalently attached to a peptide via the hydroxyls of either active agent. Codeine and promethazine are known pharmaceutical agents used in the treatment of coughs. The composition of the invention comprises codeine and promethazine covalently attached to a peptide via functional groups specified in the active agent's respective category. Codeine, guaifenesin and pseudoephidrine are used in the treatment of coughs and colds. The composition of the invention comprises codeine, guaifenesin and pseudoephidrine covalently attached to a peptide peptide via functional groups specified in the active agent's respective category. Codeine, phenylephrine and promethazine is a known pharmaceutical agent that is used in the treatment of coughs and colds. The composition of the invention comprises codeine, phenylephrine and promethazine covalently attached to a peptide via functional groups specified in the active agent's respective category.

Morphine is a known pharmaceutical agent that is used in the treatment of pain. The composition of the invention comprises morphine covalently attached to a peptide. In the present invention, morphine is covalently attached to the peptide via any of the hydroxyl groups.

Naltrexone

TABLE 77
List of Active Agents and Peptide Conjugates

| Typical Use of Active Agent | Generic Name | Peptide |
|---|---|---|
| Addiction Treatment | Naltrexone | E, K, S, ES, EW |
| Addiction Treatment | Methylnaltrexone | Glu |

### Example 88

### Naltrexone Derivatives

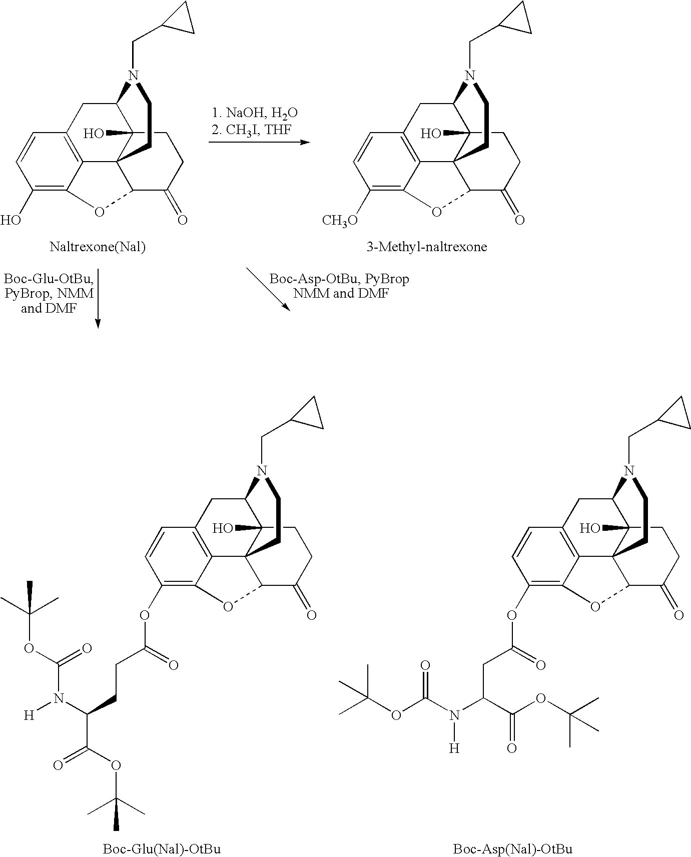 (i) Boc-Glu(Nal)-OtBu:

The solids Boc-Glu-OtBu (0.96 g, 3.18 mmol), naltrexone (1.00 g, 2.65 mmol) and PyBrop (1.73 g, 3.71 mmol) were dissolved in 5 mL of anhydrous DMF and stirred at room temperature under argon. Dry N-methylmorpholine (1.08 mL, 9.81 mmol) was added and the reaction allowed to continue stirring at room temperature under argon. After two days additional Boc-Glu-OtBu (0.096 g, 0.32 mmol), PyBrop (0.173 g, 0.37 mmol) and N-methylmorpholine (0.10 mL, 0.981 mmol) were added. After 2 more days, the solvent was removed by rotary-evaporation under high vacuum. The resulting residue was then dissolved in CHCl₃, and the resulting organic solution extracted with 2×20 mL of saturated NaCl, 3×20 mL of 10% Na₂CO₃ and a final wash with 20 mL of saturated aqueous NaCl. The organic solution was collected, dried over sodium sulfate and then adsorbed onto silica. Pure naltrexone conjugated amino acid (0.486 g, 0.78 mmol, 29%) was then isolated by flash chromatography and a gradient of 0-1.5% CH₃OH in CHCl₃. The purity of the isolated material was determined by TLC (6:1 CH₃OH:CHCl₃), and 1H NMR confirmed the presence of both the amino acid moiety and the naltrexone. ¹H NMR (360 MHz, CDCl₃): δ 6.81 (d, 1H, naltrexone aromatic), 6.63 (d, 1H, naltrexone aromatic), 4.3-4.2 (m, 1H, glutamic acid α-proton), 1.7-1.3 (pair of bs, 18H, Boc and OtBu groups.), 0.6-0.4 ppm (m, 2H, naltrexone cyclopropyl) and 0.2-0.0 ppm (m, 2H, naltrexone cyclopropyl).

Boc-Asp(Nal)-OtBu

Boc-Asp(Nal)-OtBu was obtained in 41% isolate yield using a similar protocol as the one used to prepare Boc-Glu(Nal)-OtBu. ¹H-NMR (360 MHz, CDCl₃): δ 6.84 (d, 1H, naltrexone aromatic), 6.66 (d, 1H, naltrexone aromatic), 4.6-4.5 (m, 1H, aspartic acid α-proton), 1.6-1.3 (pair of bs, 18H, Boc and OtBu groups.), 0.7-0.5 ppm (m, 2H, naltrexone cyclopropyl) and 0.4-0.1 ppm (m, 2H, naltrexone cyclopropyl). While naltrexone has a complex NMR spectrum, there are several key protons that have distinct chemical shifts and are unique to naltrexone.

NMR Characterization:

TABLE 78
Percent of Active Agent Attached to a Carrier Peptide -

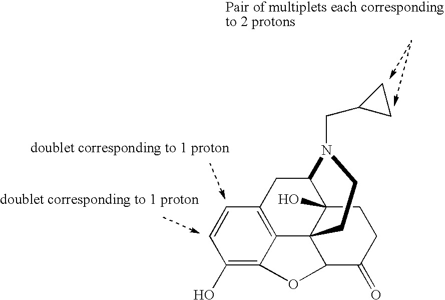

| | Lot | | | | % Active Agent | |
|---|---|---|---|---|---|---|
| Active Agent Conjugate | Number | Amount | | Yield | NMR | UV |
| pGlu(Naltrexone) 1A | TM124 | 85 | mg | | 16% | nd |
| pGlu(Naltrexone) 1B | TM124 | 95 | mg | | ˜16% | nd |
| pGlu(Naltrexone) | BBI152 | 404 | mg | 26% | 50% | 55% |
| pGlu(Naltrexone) | BBI161 | 81 | mg | <1% | 40% | nd |

nd = not determined

### Example 89

### Preparation of Carbamate Linked Naltrexone-Polymer Conjugates

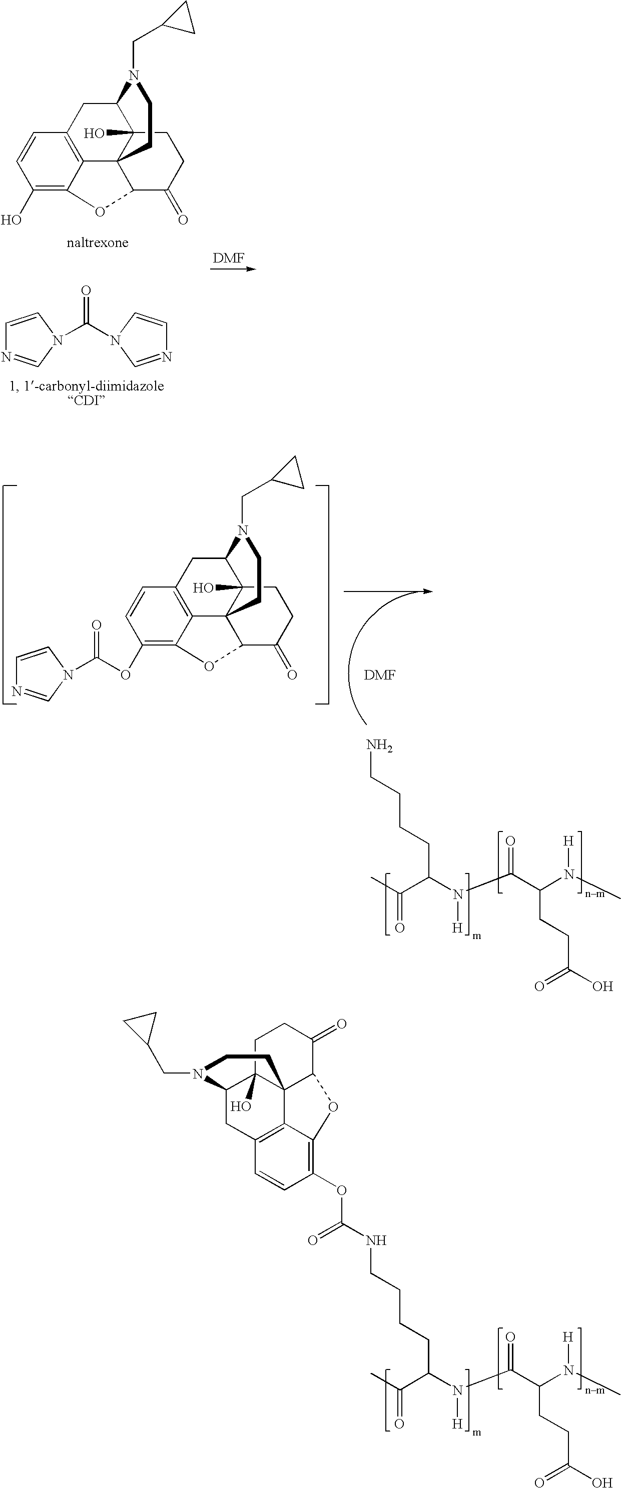

Naltrexone-hydrochloride (520 mg, 1.37 mmol) and 1-1′-carbonyl-diimidazole (CDI) (202 mg, 1.25 mmol) were dissolved in anhydrous DMF (5 mL). The reaction was then allowed to stir for 1 hour at room temperature under argon. Glutamic acid-lysine copolymer (GluₙLysₘ, 2.5 mmol of free lysine sidechains*) was then added as a suspension in 15 mL DMF, and the reaction allowed to continue stirring under argon at room temperature for 2 days. The solvent, DMF, was then removed by rotary evaporation under high vacuum, leaving a green solid. The solid was dissolved in water (20 mL), and the aqueous solution filtered/concentrated using ultrafiltration (1000 mw cutoff) to remove small molecular weight starting materials and byproducts. Two aliquots of water (10 mL each) were added and the solution filtered/concentrated after each addition to a final volume of ˜2 mL. The remaining solution was freed of solvent by rotary evaporation and the resulting solid dried over night in a vacuum chamber at room temperature. This afforded the carbamate conjugate (642 mg, 43% yield assuming saturation of available lysine sidechains) with an approximate loading of 1:4 (naltrexone/amino acid residue) as estimated by ¹H-NMR. ¹H NMR (360 MHz, DMSO-d₆): δ 6.78 and 6.61 (bs, 1H each, naltrexone-aromatic); 2.74 (bs, ˜8H, γ-Glu); 2.20 (bs, ˜8H, β-Glu), 0.50 (bs, 2H, naltrexone-cyclopropyl) and 0.16 (bs, 2H, naltrexone-cyclopropyl). (*mmol of Lysine sidechains is estimated based on a 1:1 Glu/Lys ratio as previously determined by NMR. This copolymer was prepared from Lys(Boc)-NCA and Glu(OtBu)-NCA using standard NCA polymerization methods. The resulting polymer (1.00 g, 2.5 mmol Lys) was deprotected using 4N HCl in Dioxane).

### Example 90

### Preparation of Carbonate Linked Naltrexone-Polymer Conjugates

(i) Reaction of Naltrexone (free base) with CDI

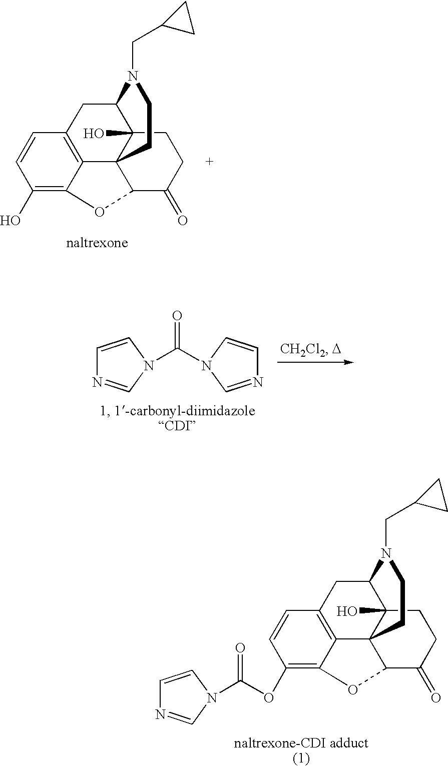

CDI (0.522 g, 3.2 mmol) was dissolved at room temperature in 20 mL of dry methylene chloride in a flask charged with argon. The naltrexone (1.00 g, 2.9 mmol) dissolved in methylene chloride (20 mL) was then added drop wise to the CDI solution. An additional 10 mL of methylene chloride was used to rinse the vessel that had contained the naltrexone, and the wash added to the reaction mixture. The reaction was heated to 50° C., and allowed to stir over night under argon at a temperature between 40 and 50° C. The solvent was then removed by rotary evaporation under high vacuum. ¹H-NMR indicated that the tacky solid contained a mixture of imidazole, the adduct 1 and unreacted starting materials. Imidazole and compound 1 were the dominant components. ¹H NMR (360 MHz, d₆-DMSO): δ 8.27 (bm, 1H, 1); 7.74 (bs, 2H, imidazole); 7.53 (t, 1H, 1); 7.24 (bs, 1H, imidazole); 7.14 (bm, 1H, 1); 6.95 (d, 1H, 1) and 6.73 (d, 1H, 1).

(ii) Reaction of Naltrexone-CDI adduct with Serₙ

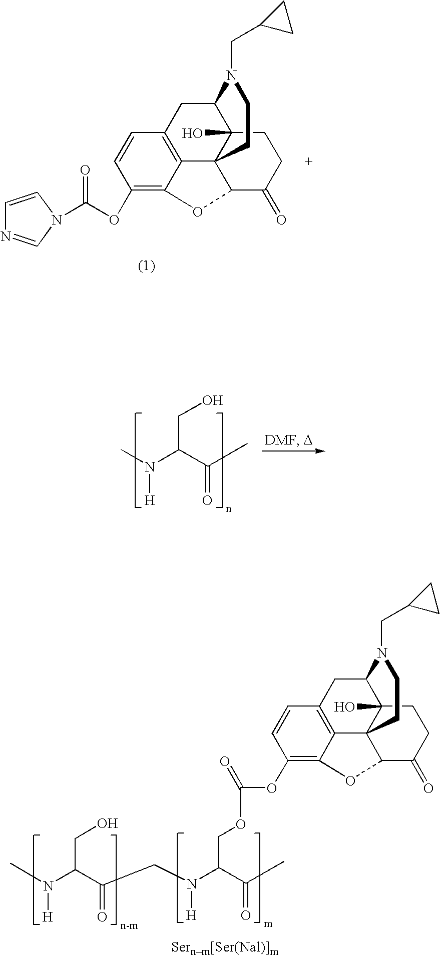

The solid from step 1 was dissolved in anhydrous N-methylpyrrolidinone (NMP), and solid Serₙ (0.51 g, 5.9 mmol) added to the solution. The reaction mixture was then heated to 60° C. under argon, and allowed to stir under argon, over night at a temperature between 50 and 60° C. The organic solution was then diluted into 100 mL of water. Precipitate formed immediately, and the solid (A) was collected by centrifuge, and the pellets then dried over night in a vacuum chamber. The water in the supernatant was removed by rotary evaporation, and the NMP solution that remained was diluted into ether (100 mL). Again, precipitate formed immediately. This solid (B) was collected by filtration and then dried over night in a vacuum chamber. Both solids were hygroscopic and appeared similar in composition by TLC (3:1 CHCl₃/CH₃OH). Therefore, solids A and B were combined and dissolved/suspended in ˜50 mL water. Ultrafiltration (1000 mw cutoff) was used to remove impurities such as unreacted naltrexone and imidazole, leaving the Serₙ and the naltrexone conjugate, Serₙ₋ₘ[Ser(Nal)]ₘ. The suspended material was washed with 5 aliquots of water (10 mL each), and then pelletted by centrifugation. The polymer conjugate was then dried over night in a vacuum chamber. This afforded 80 mg (˜5% yield) of material with an estimated loading of 1:19 naltrexone/serine (based on ¹H-NMR).

¹H NMR (360 MHz, DMSO-d₆): δ 5.03 (bs, ˜19H, α-Ser); 0.59 (bs, 2H, naltrexone-cyclopropyl) and 0.34 (bs, 2H, naltrexone-cyclopropyl).
Methyl Naltrexone-Glucose Ketal Conjugate

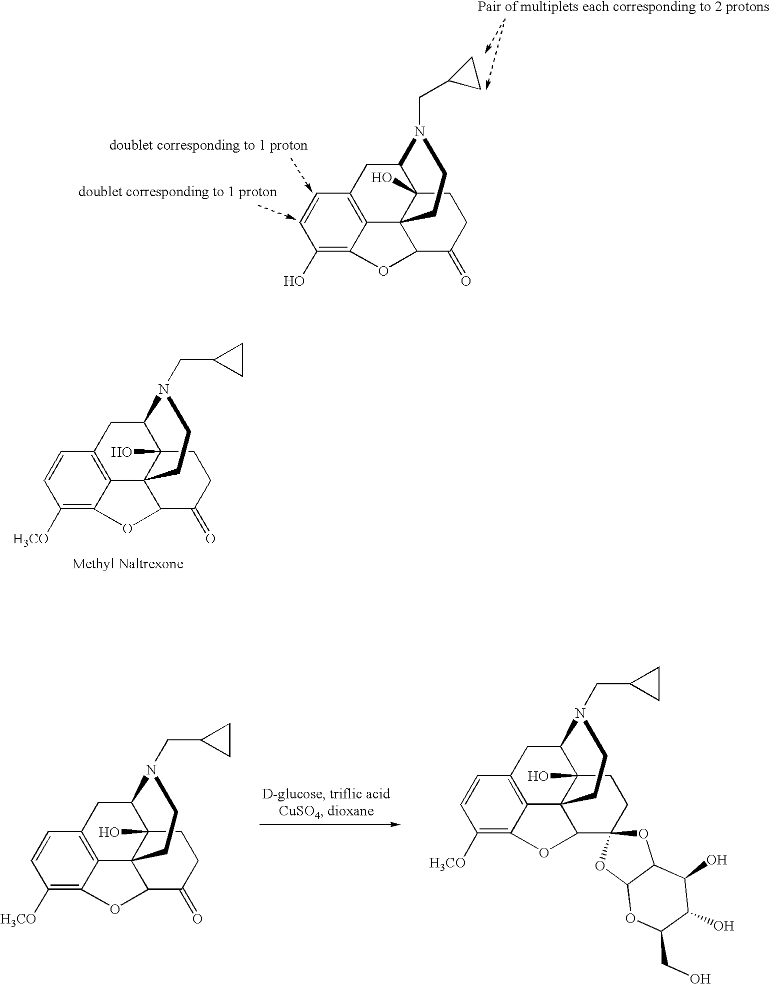

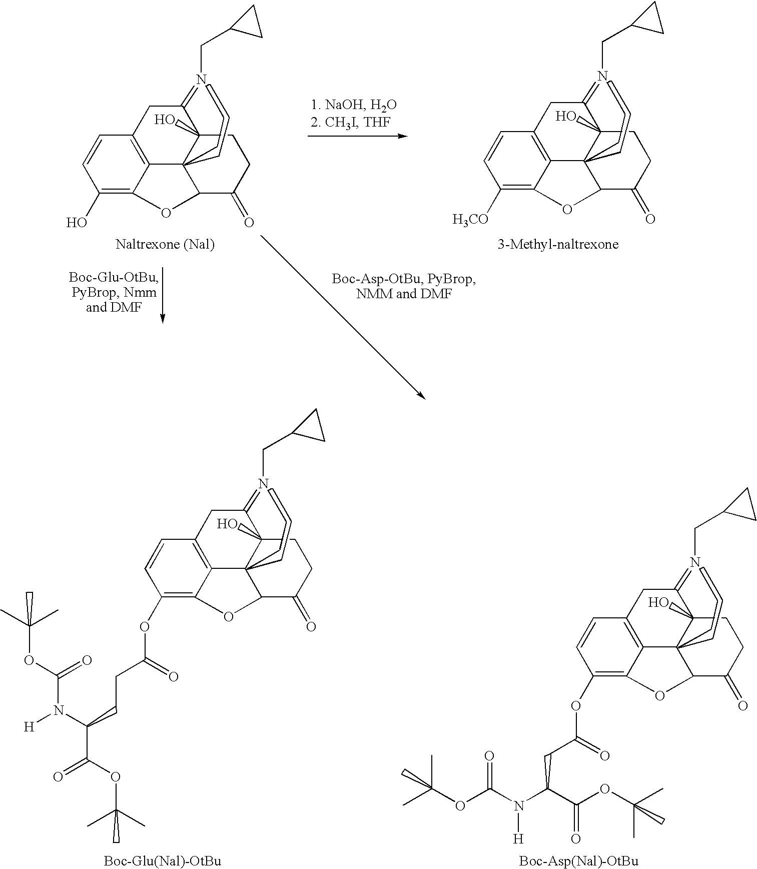

### Example 91

3-Methyl-naltrexone: Naltrexone (6.0 g, 16.5 mmol) was dissolved in 100 ml distilled water. The solution was titrated with 1N NaOH to a final pH of 11.8. In the course of the titration, neutral naltrexone precipitated from solution and then went back into solution. Upon reaching pH 11.8, the solvent was removed by rotary-evaporation under high vacuum, and the resulting solid stored under vacuum over night at room temperature. The solid was then suspended/dissolved in anhydrous tetrahydrofuran (200 ml) and allowed to stir at room temperature under argon. A solution of iodomethane (2.1 mg, 33 mmol) in 50 ml of tetrahydrofuran was added dropwise over the course 30 minutes. The reaction was then allowed to stir an additional 3 hours at room temperature under argon. The solvent was then removed by rotary-evaporation under reduced pressure. The residual solid was then dissolved in 40 ml of CHCl₃ and the organic solution washed with 30 ml of saturated NaCl, 3×30 ml of 1N NaOH and finally twice more with 30 ml saturated aqueous NaCl. The organic solution was collected and dried over sodium sulfate. Removal of solvent by rotary-evaporation and drying over night under vacuum afforded pure 3-methylnaltrexone (5.6 g, 15.8 mmol, 96% yield) as a brown residue and composition determined by TLC and ¹H-NMR. Features used to identify the compound by comparison to the spectrum of naltrexone: ¹H-NMR (360 MHz, CDCl₃) δ 6.677 (d, 1H, naltrexone aromatic), 6.591 (d, 1H, naltrexone aromatic), 3.874 (s, 3H, methoxy group.), 0.6-0.5 ppm (m, 2H, naltrexone cyclopropyl) and 0.2-0.1 ppm (m, 2H, naltrexone cyclopropyl).

### Example 92

### Methyl Naltrexone-Glucose Ketal Conjugate

To a solution of methyl naltrexone (0.200 g, 0.56 mmol) in dioxane (20 ml) was added D-α-glucose (2.02 g, 11.2 mmol), triflic acid (0.05 ml, 0.62 mmol), and CuSO_{4 (}1.00 g). The reaction mixture was stirred at ambient temperatures for 4 days. Reaction was then filtered, neutralized with NaHCO₃ (sat.) and filtered again. Dioxane and water were removed and the residue was taken up in CHCl₃ and extracted with water (3×100 ml). The organic layer was dried over MgSO₄ and solvents were removed under reduced pressure. Crude product was purified over silica gel (0-10% MeOH in CHCl₃) to obtain the ketal conjugate (0.010 g) in a 1:1 mixture with free methyl naltrexone: ¹H NMR (CDCl₃) δ 0.14 (br s, 4H, naltrexone cyclopropyl), 0.53 (br m, 4H, naltrexone cyclopropyl), 0.90 (m, 2H, naltrexone cyclopropyl), 1.48 (m, 6H, naltrexone), 2.19-2.78 (m, 12H, naltrexone), 3.03 (m, 2H, naltrexone), 3.75 (q, 2H, glucose), 3.87 (m, 8H, naltrexone CH₃ and glucose), 3.97 (q, 2H, glucose), 4.14 (q, 1H. glucose), 4.33 (t, 1H, glucose), 4.66 (s, 1H, naltrexone), 6.65 (m, 4H, naltrexone).

### Example 93

### Polyserine-Naltrexone

Naltrexone, an opoid antagonist, was chosen as a model compound for testing conjugates for the hypothesis that conjugates of opoid drugs can afford extended release, while also lowering the potential for abuse. Naltrexone is chemically similar to orally delivered analgesics such as oxycodone and hydromorphone and therefore amenable to synthesizing conjugates for testing in vitro and in vivo performance.

Synthesis

Polyserine-naltrexone (carbonate-linked) conjugates were synthesized by the following method:

1) Polymer activation. N-acetylated polyserine-methyl ester (0.69 g, 7.9 mmol) was dissolved in N-methylpyrolidinone (15 ml) and allowed to stir under argon at ambient temperature. Carbonyldiimmidazole (CDI, 1.93 g, 11.9 mmol) was added and the reaction allowed to stir over night under argon. Then, 100 ml of acetonitrile were added and the mixture allowed to sit at 4° C. for 2 hours. The precipitate that formed was collected by centrifugation and the resulting pellet then resuspended in acetonitrile. This suspension was then centrifuged and the pellet dried over night under a vacuum.

2) Tetrabutylammonium salt of naltrexone. Naltrexone hydrochloride (1.5 g, 3.979 mmol) was dissolved in water (˜50 ml) and this solution titrated with 1N LiOH to a pH of ˜11-12. Tetrabutylammonium chloride (2.6 g, 4.0 mmol) was then added. The aqueous solution was then extracted with 3 equal volumes of chloroform (20 ml each). The organic solutions were pooled and dried with magnesium sulfate. The solvent was then removed using a rotovap, and the resulting solid dried over night under a high vacuum.

3) Conjugation reaction. The solid material from step 1 was dissolved/suspended in 15 ml of N-methylpyrrolidinone and the resulting solution placed under argon. The naltrexone salt from step 2 was then added, and the reaction then allowed to warm to ˜50-60° C. The reaction was then allowed to stir two days under these conditions, at which point water was added (˜200 ml). The aqueous solution was then concentrated by ultrafiltration (1000 mw cutoff). The concentrated solution (˜5 ml) was then diluted to a volume of 50 ml with water. The aqueous solution was then titrated to pH 3 with 1N HCl and then concentrated by ultrafiltration. This process was repeated two more times. Following the final concentration, the aqueous solution (˜5 ml) was then freed of solvent using a rotovap and high vacuum. The resulting solid was then stored over night under high vacuum. This afforded 50 mg of brown solid. A serine:naltrexone ratio of approximately 1:6 (BB272) and 1:10 (BB301) was estimated by nuclear magnetic resonance (NMR). A schematic of synthesis is shown in FIG. 1.

### Example 94

### Boc-Ser(CO-Methyl Naltrexone)-OtBu

To a solution of methyl naltrexone (1.00 g, 2.82 mmol) in THF at −78° C. was added LiN(SiMe₃)₂ (1.0M in THF, 5.92 mmol) dropwise via syringe. This solution was stirred at −78° C. for 1 hour. In a separate reaction, Boc-Ser-OtBu (0.220 g, 0.84 mmol) was dissolved in THF (5 ml) with NMM (0.10 ml, 0.92 mmol) and triphosgene (0.250 g, 0.84 mmol) added. This solution was stirred at −78° C. for 30 minutes. The first reaction was added slowly to the second at −78° C. The combined reaction was allowed to warm to ambient temperature and stirred for 18 hours. After this, water (10 ml) was added. Solvent was removed and residue was partitioned between CHCl₃/water (50 ml each) and was extracted twice with CHCl₃ (50 ml). Combined organics were washed with brine (50 ml), pH 8 water (50 ml), dried with MgSO4 and solvent removed. A preparative TLC was taken (100% CHCl3). NMR of TLC material confirmed the presence of product.

The results of the examples show that conjugation of naltrexone to a polymer of serine via a carbonate linkage can prevent spiking of the drug (decrease Cₘₐₓ) and afford sustained release (increase Tₘₐₓ while maintaining approximately equal AUC).

I In Vitro and In Vivo Performance of Polyserine-Naltrexone Conjugate (Carbonate Linked)

X:A—In Vivo Performance of Polyserine-Naltrexone Conjugate (Rat Model) (Lot No. BB-272, 1:6 Naltrexone:Serine Ratio)

Polyserine-naltrexone conjugates were tested in male Sprague Dawley rats (˜250 g). Defined doses were delivered orally in gelatin capsules containing purified dry powder polyserine-naltrexone conjugates or naltrexone. No excipients were added to the capsules. Content of naltrexone in the PolySerine-Naltrexone conjugate was estimated to be 30% as based on the 1:6 ratio of naltrexone:serine determined by NMR. Polyserine-naltrexone conjugate was given to four rats at a dose of 12 mg which contained 3.6 mg of naltrexone. Doses of naltrexone (3.6 mg) equivalent to the naltrexone content of the conjugate were also given to four rats. Capsules were delivered orally to rats at time-zero using a capsule dosing syringe. Serum was collected from rats 2, 4, 6, 9, and 12 hours after capsule delivery. Serum naltrexone concentrations were determined by ELISA using a commercially available kit (Nalbuphine, product #102819, Neogen Corporation, Lansing Mich.).

TABLE 79
Serum Concentrations (ng/mL) of Individual Rats Fed;
PolySerine-Naltrexone Conjugate vs. Naltrexone

| | Polyserine-naltrexone | | | | Naltrexone | | | |
|---|---|---|---|---|---|---|---|---|
| | Rat | Rat | | | Rat | Rat | | |
| Hours | #1 | #2 | Rat #3 | Rat #4 | #1 | #2 | Rat #3 | Rat #4 |
| 2 | 58 | 35 | 22 | 22 | 33 | 91 | 37 | 22 |
| 4 | 66 | 46 | 14 | 27 | 6 | 25 | 12 | 3 |
| 6 | 34 | 21 | 11 | 26 | 13 | 10 | 8 | 6 |
| 9 | 22 | 13 | 4 | 10 | 3 | 6 | 2 | 1 |
| 12 | 8 | 16 | 3 | 5 | 1 | 2 | 1 | 2 |

TABLE 80
Mean Serum Concentrations of PolySerine-Naltrexone vs. Naltrexone

| | Polyserine-naltrexone | Naltrexone |
|---|---|---|
| Hours | (ng/ml +/− SD) | (ng/ml +/− SD) |
| 2 | 34 +/− 17 | 46 +/− 31 |
| 4 | 38 +/− 23 | 11 +/− 10 |
| 6 | 23 +/− 10 | 9 +/− 3 |
| 9 | 12 +/− 8 | 3 +/− 2 |
| 12 | 8 +/− 6 | 1 +/− 1 |

Serum levels of individual animals are shown in Table 79. Mean serum levels are shown in Table 80. Serum levels spiked earlier for naltrexone (2 hours) than for the drug administered as a polyserine-naltrexone conjugate (4 hours). Serum levels of naltrexone for the polyserine-naltrexone conjugate remained elevated considerably longer than for naltrexone. Additionally, the peak level was significantly lower for the polyserine-naltrexone conjugate. It should be noted that the 2 hour time point was the first measurement of naltrexone serum levels. Since this was the peak level measured for naltrexone it can not be determined whether or not levels peaked at a higher concentration earlier. Consequently, it was not possible to accurately determine the Cmax or area under serum concentration curve (AUC) for naltrexone in this experiment.

X:B—In Vivo performance of PolySerine-Naltrexone Conjugate

(Lot No. BB-301, 1:10 Naltrexone:Serine Ratio)

Polyserine-naltrexone conjugates were tested in Sprague-dawley rats (˜250 g). Defined doses were delivered orally in gelatin capsules containing purified dry powder polyserine-naltrexone conjugates or naltrexone. No excipients were added to the capsules. Content of naltrexone in the polyserine-naltrexone conjugate BB-272 was estimated to be 30% as based on the 1:6 ratio of naltrexone:serine determined by NMR. Polyserine-naltrexone conjugate was given to five rats at a dose of 12.9 mg which contained 3.6 mg of naltrexone. Doses equivalent to the naltrexone contained in the batch of polyserine-naltrexone (BB-301) were also given to five rats. Additionally, half the equivalent dose (1.8 mg) was given at time-zero, followed by a second half-dose at 6.5 hrs to five rats.

Capsules were delivered orally to rats at time-zero using a capsule delivery syringe. Serum was collected at 0.5, 1.5, 3, 5, 8, 12, 15 and 24 hours after capsule delivery for the polyserine-naltrexone (BB-301) and equivalent naltrexone dosed rats. Serum was collected at 0.5, 1.5, 3, 5, 8, 11.5, 14.5 and 24 hours after capsule delivery for rats dosed with half-equivalent doses at 0 and 6.5 hours. Serum naltrexone concentrations were determined by ELISA using a commercially available kit (Nalbuphine, product #102819, Neogen Corporation, Lansing Mich.).

TABLE 81
Serum Concentrations (ng/mL) of Individual Rats Fed;
PolySerine-Naltrexone Conjugate vs. Naltrexone

| | Polyserine-naltrexone | | | | | Naltrexone (equal dose) | | | | |
|---|---|---|---|---|---|---|---|---|---|---|
| Hours | Rat #1 | Rat #2 | Rat #3 | Rat #4 | Rat #5 | Rat #1 | Rat #2 | Rat #3 | Rat #4 | Rat #5 |
| 0.5 | 0 | 0 | 0 | 1 | 0 | 141 | 128 | 126 | 142 | 39 |
| 1.5 | 5 | 4 | 12 | 38 | 23 | 85 | 79 | 46 | 95 | 102 |
| 3 | 21 | 12 | 24 | 16 | 52 | 62 | 44 | 30 | 46 | 91 |
| 5 | 20 | 17 | 23 | 38 | 37 | 193 | 16 | 8 | 19 | 45 |
| 8 | 22 | 14 | 32 | 32 | 13 | 6 | 2 | 5 | 4 | 19 |
| 12 | 10 | 47 | 29 | 19 | 7 | 1 | 2 | 3 | 2 | 3 |
| 15 | 8 | 7 | 13 | 9 | 5 | 1 | 1 | 2 | 2 | 4 |
| 24 | 4 | 4 | 4 | 4 | 3 | 1 | 1 | 3 | 2 | 2 |

TABLE 82
Mean Serum Concentrations of
PolySerine-Naltrexone vs. Naltrexone (equal dose) vs. Naltrexone
(½ dose × 2)

| | Polyserine- | | |
|---|---|---|---|
| | naltrexone | Naltrexone (equal) | Naltrexone (½ × 2) |
| Hours | (ng/ml +/− SD) | (ng/ml +/− SD) | (ng/ml +/− SD) |
| 0.5 | 0 | 115 +/− 47 | 72 +/− 69 |
| 1.5 | 17 +/− 14 | 82 +/− 25 | 44 +/− 46 |
| 3 | 25 +/− 16 | 55 +/− 26 | 13 +/− 11 |
| 5 | 27 +/− 10 | 56 +/− 16 | 4 +/− 3 |
| 8 | 23 +/− 9 | 7 +/− 8 | 68 +/− 32 |
| 11.5 | NA | NA | 11 +/− 9 |
| 12 | 22 +/− 16 | 2 +/− 1 | NA |
| 14.5 | NA | NA | 10 +/− 3 |
| 15 | 8 +/− 3 | 2 +/− 1 | NA |
| 24 | 4 +/− 0.4 | 2 +/− 1 | 6 +/− 1 |

Serum levels of individual animals are shown in Table 81. Mean serum levels are shown in Table 82. Naltrexone serum levels spiked earlier (0.5 hours) for naltrexone than for the drug administered as a polyserine-naltrexone conjugate (5 hours). Serum levels of naltrexone for the polyserine-naltrexone conjugate remained elevated considerably longer (>12 hours) than for the monomeric naltrexone control (<8 h). Serum concentration curves crossed at approximately 7 hours. Additionally, the mean of the peak level concentration (Cmax) was significantly lower for the conjugated naltrexone. Further, the mean time to peak concentration (Tmax) was significantly longer for the polyserine-naltrexone conjugate. The mean AUC of the polyserine-naltrexone conjugate was approximately 75% of the naltrexone mean AUC. Statistically the mean AUCs were not significantly different (P<0.05). Serum levels of rats fed one-half-dose (1.8 mg) at time zero and at 6.5 hours were compared to those of rats fed polyserine-naltrexone conjugate. Concentration levels remained elevated for the conjugate past those for the second naltrexone dose, with the curves crossing at approximately 2.5 hours and again at approximately 11 hours (double cross-over of the serum concentration curves).

TABLE 83
Mean Pharmacokinetic Parameters of Polyserine-Naltrexone vs.
Naltrexone

| | Cmax +/− SD | Tmax +/− SD | AUC 0-24 h +/− SD |
|---|---|---|---|
| Dosage Form | (ng/ml) | (hours) | (ng h/ml) |
| Polyserine- | 38.2 +/− 11.9 | 7.3 +/− 3.1 | 356 +/− 66 |
| naltrexone | | | |
| Naltrexone | 124.5 +/− 16.6 | 0.75 +/− 0.5 | 477 +/− 183 |

X:C—In Situ Performance of Polyserine-Naltrexone-Caco-2 Cell Digestion

Polyserine-naltrexone conjugates BB-272 and BB-301 were incubated with monolayers of Caco-2 cells for 4 hours in phosphate buffered saline. Buffer was removed from the monlayers and concentrated on SP-18 columns. Concentrated samples were analyzed for the presence of naltrexone by reverse phase HPLC. Each Polyserine-naltrexone conjugate showed significant release of free naltrexone from the polymer conjugate in three separate samples. In conclusion, Caco-2 cellular enzymes affected release of naltrexone from Polyserine-naltrexone conjugates BB-272 and BB-301. Release of carbonate linked drug from a conjugate by intestinal cellular enzymes affords a mechanism for drug absorption following oral administration.

X:D—Treatment of Polyserine-Naltrexone Conjugates with Intestinal Enzymes

Polyserine-naltrexone (BB-272 and BB-301) were treated with enzymes found in the stomach and lumen of the small intestines. The enzymes tested, which included pepsin, pancreatic lipase, and pancreatin were ineffective in releasing naltrexone from the polyserine-naltrexone conjugates. Other enzymes, including protease and amidase, also did not affect drug release. These results suggest that polyserine-naltrexone is resistant to enzymes found in the stomach and lumen of the intestine.

X:E—Conclusion

In conclusion, conjugation of naltrexone to a polymer of serine via carbonate linkage comprised a pharmaceutical composition that afforded extended release when administered orally. The said conjugates were resistant to a number of enzymes found in the luminal fluids of the intestinal tract. In contrast, incubation of the compositions with Caco-2 human intestinal epithelial cells affected release of naltrexone. In a specific embodiment of the invention, pharmaceutical compositions comprised of a drug covalently bound to a carrier that are resistant to luminal enzymes and depend on intestinal cell associated enzymes for drug release afford extended release characteristics to the bound drug.

Butorphanol is a known pharmaceutical agent that is used in the treatment of pain. It is both commercially available and readily manufactured using published synthetic schemes by those of ordinary skill in the art. In the present invention, butorphanol is covalently attached to the peptide via the phenyl hydroxyl group.

Dihydrocodeine is a known pharmaceutical agent that is used in the treatment of pain. The composition of the invention comprises dihydrocodeine covalently attached to a peptide. In the present invention, dihydrocodeine is covalently attached to the peptide via the hydroxyl group.

Dihydromorphine is a known pharmaceutical agent that is used in the treatment of pain. The composition of the invention comprises dihydromorphine covalently attached to a peptide. In the present invention, dihydromorphine is covalently attached to the peptide via the hydroxyl group.

Ethylmorphine is a known pharmaceutical agent that is used in the treatment of pain. The composition of the invention comprises ethylmorphine covalently attached to a peptide. In the present invention, ethylmorphine is covalently attached to the peptide via the hydroxyl group

Methyldihydromorphinone is a known pharmaceutical agent that is used in the treatment of pain. The composition of the invention comprises methyldihydromorphinone covalently attached to a peptide. In the present invention, methyldihydromorphinone is covalently attached to the peptide via the hydroxyl group

1. A pharmaceutical composition comprising an opioid covalently bound to a peptide carrier or a pharmaceutically acceptable salt thereof and at least one pharmaceutically acceptable additive in a form suitable for oral administration, wherein said opioid covalently bound to a peptide carrier or salt thereof is in an amount sufficient to provide a therapeutically effective amount of said opioid, but at a reduced rate of absorption of the opioid as compared to unbound opioid.

2. A pharmaceutical composition comprising an opioid covalently bound to a peptide carrier or a pharmaceutically acceptable salt thereof in an oral dosage form that provides an AUC comparable to an extended release product.

3. A composition for reducing drug abuse comprising an opioid covalently bound to a peptide carrier or a pharmaceutically acceptable salt thereof in an oral dosage form wherein said opioid is not released following attempted disruption of the covalently-bonded opioid formulation prior to ingestion.

4. The composition of claim 1 wherein said carrier peptide is between 1 and 10 amino acids.

5. The composition of claim 1 wherein the said amino acid or peptide is comprised of one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine.

6. The composition of claim 1 wherein the opioid is hydrocodone, oxycodone, hydromorphone, oxymorphone, codeine, morphine, naltrexone, butorphanol, dihydrocodeine, dihydromorphine, ethylmorphine, or methyldihydromorphinone.

7. A method for reducing the abuse potential of an opioid composition comprising orally administering the composition of claim 1 to a human in need thereof.

8. A method for preventing a euphoric effect of an opioid comprising orally administering the composition of claim 1 while still providing a therapeutically bioequivalent AUC.

9. A method of treating acute or chronic pain comprising administering to a patient the composition of claim 1.
